SEQUENCE LISTING (212 sequences)

SEQ ID NO: 1 - protein, 5 aa, Homo sapiens
VLQRY

SEQ ID NO: 2 - protein, 8 aa, Homo sapiens
TKVNFYAW

SEQ ID NO: 3 - protein, 9 aa, Homo sapiens
SGLRSLTTL

SEQ ID NO: 4 - protein, 6 aa, Homo sapiens
SNFLRG

SEQ ID NO: 5 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVEGLRSLTTLLRALGAQKEAISPPDAASAAPLRTITADTFRKLFRVYSNFLRGKLKLYTGEACRTGDR

SEQ ID NO: 6 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTDVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 7 - DNA, 580 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcactgcatgtggataaagccgtcagtggccttcgcagcctcaccactctgcttcgggctctgggagcccagaaggaagccatctcccctccagatgcggcctcagctgctccactccgaacaatcactgctgacactttcgcaaactcttccgagtctactccaatttcctccggggaaagctgaagctgtacacaggggaggcctgcaggacaggggacagatga

SEQ ID NO: 8 - DNA, 35 bp, Artificial
  Description of Artificial Sequence primer
agctctcgaggcgcggagatgggggtgcacgaatg

SEQ ID NO: 9 - DNA, 36 bp, Artificial
  Description of Artificial Sequence primer
atgctctagacacacctggtcatctgtcccctgtcc

SEQ ID NO: 10 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 11 - DNA, 45 bp, Artificial
  Description of Artificial Sequence primer
catgtggataaagccgtcgagggccttcgcagcctcaccactctg

SEQ ID NO: 12 - DNA, 45 bp, Artificial
  Description of Artificial Sequence primer
cagagtggtgaggctgcgaaggccctcgacggctttatccacatg

SEQ ID NO: 13 - DNA, 45 bp, Artificial
  Description of Artificial Sequence primer
gagaatatcactgtcccagacaccgacgttaatttctatgcctgg

SEQ ID NO: 14 - DNA, 45 bp, Artificial
  Description of Artificial Sequence primer
ccaggcatagaaattaacgtcggtgtctgggacagtgatattctc

SEQ ID NO: 15 - protein, 193 aa, Artificial
  Description of Artificial Sequence mutein
MGVHECPAWLWLLLSLLSLPLGLPVLGAPPRLACDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 16 - protein, 193 aa, Artificial
  Description of Artificial Sequence mutein
MGVHECPAWLWLLLSLLSLPLGLPVLGAPPRLIADSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 17 - protein, 193 aa, Artificial
  Description of Artificial Sequence mutein
MGVHECPAWLWLLLSLLSLPLGLPVLGAPPRLICDSIVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 18 - protein, 193 aa, Artificial
  Description of Artificial Sequence mutein
MGVHECPAWLWLLLSLLSLPLGLPVLGAPPRLICDSRSLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 19 - protein, 193 aa, Artificial
  Description of Artificial Sequence mutein
MGVHECPAWLWLLLSLLSLPLGLPVLGAPPRLICDSRVAERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 20 - protein, 193 aa, Artificial
  Description of Artificial Sequence mutein
MGVHECPAWLWLLLSLLSLPLGLPVLGAPPRLICDSRVLARYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 21 - protein, 193 aa, Artificial
  Description of Artificial Sequence mutein
MGVHECPAWLWLLLSLLSLPLGLPVLGAPPRLICDSRVLEAYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 22 - protein, 193 aa, Artificial; identical to SEQ ID NO: 10
  Description of Artificial Sequence mutein

SEQ ID NO: 23 - protein, 193 aa, Artificial
  Description of Artificial Sequence mutein
MGVHECPAWLWLLLSLLSLPLGLPVLGAPPRLICDSRVLEEYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 24 - protein, 193 aa, Artificial
  Description of Artificial Sequence mutein
MGVHECPAWLWLLLSLLSLPLGLPVLGAPPRLICDSRVLEQYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 25 - protein, 193 aa, Artificial
  Description of Artificial Sequence mutein
MGVHECPAWLWLLLSLLSLPLGLPVLGAPPRLICDSRVLERALLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 26 - protein, 193 aa, Artificial
  Description of Artificial Sequence mutein
MGVHECPAWLWLLLSLLSLPLGLPVLGAPPRLICDSRVLERFLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 27 - protein, 193 aa, Artificial
  Description of Artificial Sequence mutein
MGVHECPAWLWLLLSLLSLPLGLPVLGAPPRLICDSRVLERILLEAEEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 28 - protein, 193 aa, Artificial
  Description of Artificial Sequence mutein
MGVHECPAWLWLLLSLLSLPLGLPVLGAPPRLICDSRVLERYLLEAEEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 29 - protein, 193 aa, Artificial
  Description of Artificial Sequence mutein
MGVHECPAWLWLLLSLLSLPLGLPVLGAPPRLICDSRVLERYLLEAAEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 30 - protein, 193 aa, Artificial
  Description of Artificial Sequence mutein
MGVHECPAWLWLLLSLLSLPLGLPVLGAPPRLICDSRVLERYLLEAKAAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 31 - protein, 193 aa, Artificial
  Description of Artificial Sequence mutein
MGVHECPAWLWLLLSLLSLPLGLPVLGAPPRLICDSRVLERYLLEAKEAEKITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 32 - protein, 193 aa, Artificial
  Description of Artificial Sequence mutein
MGVHECPAWLWLLLSLLSLPLGLPVLGAPPRLICDSRVLERYLLEAKEAENITTGSAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 33 - protein, 193 aa, Artificial
  Description of Artificial Sequence mutein
MGVHECPAWLWLLLSLLSLPLGLPVLGAPPRLICDSRVLERYLLEAKEAENITTGYAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 34 - protein, 193 aa, Artificial
  Description of Artificial Sequence mutein
MGVHECPAWLWLLLSLLSLPLGLPVLGAPPRLICDSRVLERYLLEAKEAENITTGCNEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 35 - protein, 193 aa, Artificial
  Description of Artificial Sequence mutein
MGVHECPAWLWLLLSLLSLPLGLPVLGAPPRLICDSRVLERYLLEAKEAENITTGCAETCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 36 - protein, 193 aa, Artificial
  Description of Artificial Sequence mutein
MGVHECPAWLWLLLSLLSLPLGLPVLGAPPRLICDSRVLERYLLEAKEAENITTGCAEHSSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 37 - protein, 193 aa, Artificial
  Description of Artificial Sequence mutein
MGVHECPAWLWLLLSLLSLPLGLPVLGAPPRLICDSRVLERYLLEAKEAENITTGCAEHYSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 38 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNEKITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 39 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVKSSQPWEPLQLHVDKAVSGLRSLTTLLRALGAQKEAISPPDAASAAPLRTITADTFRKLFRVYSNFLRGKLKLYTGEACRTGDR

SEQ ID NO: 40 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVNDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 41 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVADTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 42 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPATKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 43 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDIKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 44 - protein, 193 aa, Artificial; identical to SEQ ID NO: 6
  Description of Artificial Sequence mutein

SEQ ID NO: 45 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTAVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 46 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKANFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 47 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVAFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 48 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNIYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 49 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNAYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 50 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFAAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 51 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFSAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 52 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAFKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 53 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYANKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 54 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWARMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 55 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQNAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 56 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVTVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 57 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGSALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 58 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALASEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 59 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVRKAVSGLRSLTTLLRALGAQKEAISPPDAASAAPLRTITADTFRKLFRVYSNFLRGKLKLYTGEACRTGDR

SEQ ID NO: 60 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDAAVSGLRSLTTLLRALGAQKEAISPPDAASAAPLRTITADTFRKLFRVYSNFLRGKLKLYTGEACRTGDR

SEQ ID NO: 61 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVRGLRSLTTLLRALGAQKEAISPPDAASAAPLRTITADTFRKLFRVYSNFLRGKLKLYTGEACRTGDR

SEQ ID NO: 62 - protein, 193 aa, Artificial; identical to SEQ ID NO: 5
  Description of Artificial Sequence mutein

SEQ ID NO: 63 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVAGLRSLTTLLRALGAQKEAISPPDAASAAPLRTITADTFRKLFRVYSNFLRGKLKLYTGEACRTGDR

SEQ ID NO: 64 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVTGLRSLTTLLRALGAQKEAISPPDAASAAPLRTITADTFRKLFRVYSNFLRGKLKLYTGEACRTGDR

SEQ ID NO: 65 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSALRSLTTLLRALGAQKEAISPPDAASAAPLRTITADTFRKLFRVYSNFLRGKLKLYTGEACRTGDR

SEQ ID NO: 66 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSILRSLTTLLRALGAQKEAISPPDAASAAPLRTITADTFRKLFRVYSNFLRGKLKLYTGEACRTGDR

SEQ ID NO: 67 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGARSLTTLLRALGAQKEAISPPDAASAAPLRTITADTFRKLFRVYSNFLRGKLKLYTGEACRTGDR

SEQ ID NO: 68 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLASLTTLLRALGAQKEAISPPDAASAAPLRTITADTFRKLFRVYSNFLRGKLKLYTGEACRTGDR

SEQ ID NO: 69 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLESLTTLLRALGAQKEAISPPDAASAAPLRTITADTFRKLFRVYSNFLRGKLKLYTGEACRTGDR

SEQ ID NO: 70 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRALTTLLRALGAQKEAISPPDAASAAPLRTITADTFRKLFRVYSNFLRGKLKLYTGEACRTGDR

SEQ ID NO: 71 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRILTTLLRALGAQKEAISPPDAASAAPLRTITADTFRKLFRVYSNFLRGKLKLYTGEACRTGDR

SEQ ID NO: 72 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSATTLLRALGAQKEAISPPDAASAAPLRTITADTFRKLFRVYSNFLRGKLKLYTGEACRTGDR

SEQ ID NO: 73 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLATLLRALGAQKEAISPPDAASAAPLRTITADTFRKLFRVYSNFLRGKLKLYTGEACRTGDR

SEQ ID NO: 74 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLITLLRALGAQKEAISPPDAASAAPLRTITADTFRKLFRVYSNFLRGKLKLYTGEACRTGDR

SEQ ID NO: 75 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTALLRALGAQKEAISPPDAASAAPLRTITADTFRKLFRVYSNFLRGKLKLYTGEACRTGDR

SEQ ID NO: 76 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTLLLRALGAQKEAISPPDAASAAPLRTITADTFRKLFRVYSNFLRGKLKLYTGEACRTGDR

SEQ ID NO: 77 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTKLRALGAQKEAISPPDAASAAPLRTITADTFRKLFRVYSNFLRGKLKLYTGEACRTGDR

SEQ ID NO: 78 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTALRALGAQKEAISPPDAASAAPLRTITADTFRKLFRVYSNFLRGKLKLYTGEACRTGDR

SEQ ID NO: 79 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTSLRALGAQKEAISPPDAASAAPLRTITADTFRKLFRVYSNFLRGKLKLYTGEACRTGDR

SEQ ID NO: 80 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQAEAISPPDAASAAPLRTITADTFRKLFRVYSNFLRGKLKLYTGEACRTGDR

SEQ ID NO: 81 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAAAAAPLRTITADTFRKLFRVYSNFLRGKLKLYTGEACRTGDR

SEQ ID NO: 82 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRAITADTFRKLFRVYSNFLRGKLKLYTGEACRTGDR

SEQ ID NO: 83 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTATADTFRKLFRVYSNFLRGKLKLYTGEACRTGDR

SEQ ID NO: 84 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTIAADTFRKLFRVYSNFLRGKLKLYTGEACRTGDR

SEQ ID NO: 85 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRALFRVYSNFLRGKLKLYTGEACRTGDR

SEQ ID NO: 86 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLIRVYSNFLRGKLKLYTGEACRTGDR

SEQ ID NO: 87 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFAVYSNFLRGKLKLYTGEACRTGDR

SEQ ID NO: 88 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYANFLRGKLKLYTGEACRTGDR

SEQ ID NO: 89 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSKFLRGKLKLYTGEACRTGDR

SEQ ID NO: 90 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSAFLRGKLKLYTGEACRTGDR

SEQ ID NO: 91 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNYLRGKLKLYTGEACRTGDR

SEQ ID NO: 92 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNALRGKLKLYTGEACRTGDR

SEQ ID NO: 93 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFARGKLKLYTGEACRTGDR

SEQ ID NO: 94 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLAGKLKLYTGEACRTGDR

SEQ ID NO: 95 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLEGKLKLYTGEACRTGDR

SEQ ID NO: 96 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRAKLKLYTGEACRTGDR

SEQ ID NO: 97 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGALKLYTGEACRTGDR

SEQ ID NO: 98 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGWLKLYTGEACRTGDR

SEQ ID NO: 99 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKAKLYTGEACRTGDR

SEQ ID NO: 100 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLALYTGEACRTGDR

SEQ ID NO: 101 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKAYTGEACRTGDR

SEQ ID NO: 102 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTAEACRTGDR

SEQ ID NO: 103 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEASRTGDR

SEQ ID NO: 104 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEAARTGDR

SEQ ID NO: 105 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACATGDR

SEQ ID NO: 106 - protein, 192 aa, Artificial
  Description of Artificial Sequence mutein
MGVHECPAWLWLLLSLLSLPLGLPVLGAPPRLICDSRVLERYLLEAKEAENITTGCEHCSLNENITVPDTDVNFYAWKRMEVGQQAVEVWQGLALLSEAVLRGQALLVNSSQPWEPLQLHVDKAVEGLRSLTTLLRALGAQKEAISPPDAASAAPLRTITADTFRKLFRVYSNFLRGKLKLYTGEACRTGDR

SEQ ID NO: 107 - protein, 193 aa, Artificial
  Description of Artificial Sequence mutein
MGVHECPAWLWLLLSLLSLPLGLPVLGAPPRLICDSRVLERYLLEAKEAENITTGCNETCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 108 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTDVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLEGKLKLYTGEACRTGDR

SEQ ID NO: 109 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLESLTTSLRALGAQKEAISPPDAASAAPLRTITADTFRKLFRVYSNFLRGKLKLYTGEACRTGDR

SEQ ID NO: 110 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWARMEVGQQAVEVWQGLALLSEAVLRGQALLVNSSQPWEPLQLHVDKAVSGLRSLTTLLRALGAQKEAISPPDAASAAPLRTITADTFRALFRVYSNFLRGKLKLYTGEACRTGDR

SEQ ID NO: 111 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTAVNFYAWARMEVGQQAVEVWQGLALLSEAVLRGQALLVNSSQPWEPLQLHVDKAVSGLRSLTTLLRALGAQKEAISPPDAASAAPLRTITADTFRALFRVYSNFLRGKLKLYTGEACRTGDR

SEQ ID NO: 112 - protein, 193 aa, Artificial; identical to SEQ ID NO: 10
  Description of Artificial Sequence mutein

SEQ ID NO: 113 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDAAVSGLRSLTTLLRALGAQKEAISPPDAASAAPLRTITADTFRKLFRVYSNFLRGALKLYTGEACRTGDR

SEQ ID NO: 114 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTAVNFYAWKRMEVGQQAVEVWQGLALLSEAVLRGQALLVNSSQPWEPLQLHVDAAVSGLRSLTTLLRALGAQKEAISPPDAASAAPLRTITADTFRKLFRVYSNFLRGALKLYTGEACRTGDR

SEQ ID NO: 115 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTAVNFYAWARMEVGQQAVEVWQGLALLSEAVLRGQALLVNSSQPWEPLQLHVDAAVSGLRSLTTLLRALGAQKEAISPPDAASAAPLRTITADTFRALFRVYSNFLRGALKLYTGEACRTGDR

SEQ ID NO: 116 - protein, 193 aa, Artificial
  Description of Artificial Sequence mutein
MGVHECPAWLWLLLSLLSLPLGLPVLGAPPRLICDSRVLERYLLEAKEAENITTGCAEHCSLNENITVPDTAVNFYAWARMEVGQQAVEVWQGLALLSEAVLRGQALLVNSSQPWEPLQLHVDAAVSGLRSLTTLLRALGAQKEAISPPDAASAAPLRTITADTFRALFRVYSNFLRGALALYTGEACRTGDR

SEQ ID NO: 117 - protein, 193 aa, Artificial
  Description of Artificial Sequence mutein
MGVHECPAWLWLLLSLLSLPLGLPVLGAPPRLICDSRVLERYLLEAKEAEKITTGCAEHCSLNEKITVPDTKVNFYAWKRMEVGQQAVEVWQGLALLSEAVLRGQALLVKSSQPWEPLQLHVDKAVSGLRSLTTLLRALGAQKEAISPPDAASAAPLRTITADTFRKLFRVYSNFLRGKLKLYTGEACRTGDR

SEQ ID NO: 118 - protein, 193 aa, Artificial
  Description of Artificial Sequence mutein
MGVHECPAWLWLLLSLLSLPLGLPVLGAPPRLICDSRVLERALLEAKEAEKITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 119 - protein, 193 aa, Artificial
  Description of Artificial Sequence mutein
MGVHECPAWLWLLLSLLSLPLGLPVLGAPPRLICDSRVLEAALLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 120 - DNA, 36 bp, Artificial
  Description of Artificial Sequence primer
gtctactccaatttcctcgagggaaagctgaagctg

SEQ ID NO: 121 - DNA, 34 bp, Artificial
  Description of Artificial Sequence primer
gcttcagctttccctcgaggaaattggagtagac

SEQ ID NO: 122 - DNA, 32 bp, Artificial
  Description of Artificial Sequence primer
ccgtcagtggccttgagagcctcaccactctg

SEQ ID NO: 123 - DNA, 32 bp, Artificial
  Description of Artificial Sequence primer
cagagtggtgaggctctcaaggccactgacgg

SEQ ID NO: 124 - DNA, 32 bp, Artificial; identical to SEQ ID NO: 122
  Description of Artificial Sequence primer

SEQ ID NO: 125 - DNA, 32 bp, Artificial; identical to SEQ ID NO: 123
  Description of Artificial Sequence primer

SEQ ID NO: 126 - DNA, 31 bp, Artificial
  Description of Artificial Sequence primer
cgcagcctcaccacttcgcttcgggctctgg

SEQ ID NO: 127 - DNA, 31 bp, Artificial
  Description of Artificial Sequence primer
ccagagcccgaagcgaagtggtgaggctgcg

SEQ ID NO: 128 - DNA, 40 bp, Artificial
  Description of Artificial Sequence primer
gaatatcactgtcccagacggtggtgcctggaagaggatg

SEQ ID NO: 129 - DNA, 40 bp, Artificial
  Description of Artificial Sequence primer
catcctcttccaggcaccaccgtctgggacagtgatattc

SEQ ID NO: 130 - DNA, 33 bp, Artificial
  Description of Artificial Sequence primer
tacctcttggaggccgcggaggccgagaatatc

SEQ ID NO: 131 - DNA, 33 bp, Artificial
  Description of Artificial Sequence primer
gatattctcggcctccgcggcctccaagaggta

SEQ ID NO: 132 - DNA, 35 bp, Artificial
  Description of Artificial Sequence primer
gctgacactttccgcgcactcttccgagtctactc

SEQ ID NO: 133 - DNA, 35 bp, Artificial
  Description of Artificial Sequence primer
gagtagactcggaagagtgcgcggaaagtgtcagc

SEQ ID NO: 134 - DNA, 33 bp, Artificial
  Description of Artificial Sequence primer
atttcctccggggagcgctgaagctgtacacag

SEQ ID NO: 135 - DNA, 33 bp, Artificial
  Description of Artificial Sequence primer
ctgtgtacagcttcagcgctccccggaggaaat

SEQ ID NO: 136 - DNA, 32 bp, Artificial
  Description of Artificial Sequence primer
ctccggggaaagctggcgctgtacacagggga

SEQ ID NO: 137 - DNA, 32 bp, Artificial
  Description of Artificial Sequence primer
tcccctgtgtacagcgccagctttccccggag

SEQ ID NO: 138 - DNA, 35 bp, Artificial
  Description of Artificial Sequence primer
actgtcccagacaccgcagttaatttctatgcctg

SEQ ID NO: 139 - DNA, 35 bp, Artificial
  Description of Artificial Sequence primer
caggcatagaaattaactgcggtgtctgggacagt

SEQ ID NO: 140 - DNA, 35 bp, Artificial
  Description of Artificial Sequence primer
agttaatttctatgcctgggcgaggatggaggtcg

SEQ ID NO: 141 - DNA, 35 bp, Artificial
  Description of Artificial Sequence primer
cgacctccatcctcgcccaggcatagaaattaact

SEQ ID NO: 142 - DNA, 33 bp, Artificial
  Description of Artificial Sequence primer
tgcagctgcatgtggatgcagccgtcagtggcc

SEQ ID NO: 143 - DNA, 33 bp, Artificial
  Description of Artificial Sequence primer
ggccactgacggctgcatccacatgcagctgca

SEQ ID NO: 144 - DNA, 32 bp, Artificial
  Description of Artificial Sequence primer
ctctgggagcccaggcggaagccatctcccct

SEQ ID NO: 145 - DNA, 32 bp, Artificial
  Description of Artificial Sequence primer
aggggagatggcttccgcctgggctcccagag

SEQ ID NO: 146 - DNA, 35 bp, Artificial; identical to SEQ ID NO: 132
  Description of Artificial Sequence primer

SEQ ID NO: 147 - DNA, 35 bp, Artificial; identical to SEQ ID NO: 133
  Description of Artificial Sequence primer

SEQ ID NO: 148 - DNA, 35 bp, Artificial; identical to SEQ ID NO: 140
  Description of Artificial Sequence primer

SEQ ID NO: 149 - DNA, 35 bp, Artificial; identical to SEQ ID NO: 141
  Description of Artificial Sequence primer

SEQ ID NO: 150 - DNA, 35 bp, Artificial; identical to SEQ ID NO: 132
  Description of Artificial Sequence primer

SEQ ID NO: 151 - DNA, 35 bp, Artificial; identical to SEQ ID NO: 133
  Description of Artificial Sequence primer

SEQ ID NO: 152 - DNA, 35 bp, Artificial; identical to SEQ ID NO: 140
  Description of Artificial Sequence primer

SEQ ID NO: 153 - DNA, 35 bp, Artificial; identical to SEQ ID NO: 141
  Description of Artificial Sequence primer

SEQ ID NO: 154 - DNA, 35 bp, Artificial; identical to SEQ ID NO: 138
  Description of Artificial Sequence primer

SEQ ID NO: 155 - DNA, 35 bp, Artificial; identical to SEQ ID NO: 139
  Description of Artificial Sequence primer

SEQ ID NO: 156 - DNA, 33 bp, Artificial; identical to SEQ ID NO: 142
  Description of Artificial Sequence primer

SEQ ID NO: 157 - DNA, 33 bp, Artificial; identical to SEQ ID NO: 143
  Description of Artificial Sequence primer

SEQ ID NO: 158 - DNA, 33 bp, Artificial; identical to SEQ ID NO: 134
  Description of Artificial Sequence primer

SEQ ID NO: 159 - DNA, 33 bp, Artificial; identical to SEQ ID NO: 135
  Description of Artificial Sequence primer

SEQ ID NO: 160 - DNA, 33 bp, Artificial; identical to SEQ ID NO: 142
  Description of Artificial Sequence primer

SEQ ID NO: 161 - DNA, 33 bp, Artificial; identical to SEQ ID NO: 143
  Description of Artificial Sequence primer

SEQ ID NO: 162 - DNA, 33 bp, Artificial; identical to SEQ ID NO: 134
  Description of Artificial Sequence primer

SEQ ID NO: 163 - DNA, 33 bp, Artificial; identical to SEQ ID NO: 135
  Description of Artificial Sequence primer

SEQ ID NO: 164 - DNA, 35 bp, Artificial; identical to SEQ ID NO: 138
  Description of Artificial Sequence primer

SEQ ID NO: 165 - DNA, 35 bp, Artificial; identical to SEQ ID NO: 139
  Description of Artificial Sequence primer

SEQ ID NO: 166 - DNA, 33 bp, Artificial; identical to SEQ ID NO: 142
  Description of Artificial Sequence primer

SEQ ID NO: 167 - DNA, 33 bp, Artificial; identical to SEQ ID NO: 143
  Description of Artificial Sequence primer

SEQ ID NO: 168 - DNA, 33 bp, Artificial; identical to SEQ ID NO: 134
  Description of Artificial Sequence primer

SEQ ID NO: 169 - DNA, 33 bp, Artificial; identical to SEQ ID NO: 135
  Description of Artificial Sequence primer

SEQ ID NO: 170 - DNA, 35 bp, Artificial; identical to SEQ ID NO: 138
  Description of Artificial Sequence primer

SEQ ID NO: 171 - DNA, 35 bp, Artificial; identical to SEQ ID NO: 139
  Description of Artificial Sequence primer

SEQ ID NO: 172 - DNA, 35 bp, Artificial; identical to SEQ ID NO: 140
  Description of Artificial Sequence primer

SEQ ID NO: 173 - DNA, 35 bp, Artificial; identical to SEQ ID NO: 141
  Description of Artificial Sequence primer

SEQ ID NO: 174 - DNA, 33 bp, Artificial; identical to SEQ ID NO: 142
  Description of Artificial Sequence primer

SEQ ID NO: 175 - DNA, 33 bp, Artificial; identical to SEQ ID NO: 143
  Description of Artificial Sequence primer

SEQ ID NO: 176 - DNA, 33 bp, Artificial; identical to SEQ ID NO: 134
  Description of Artificial Sequence primer

SEQ ID NO: 177 - DNA, 33 bp, Artificial; identical to SEQ ID NO: 135
  Description of Artificial Sequence primer

SEQ ID NO: 178 - DNA, 35 bp, Artificial; identical to SEQ ID NO: 138
  Description of Artificial Sequence primer

SEQ ID NO: 179 - DNA, 35 bp, Artificial; identical to SEQ ID NO: 139
  Description of Artificial Sequence primer

SEQ ID NO: 180 - DNA, 35 bp, Artificial; identical to SEQ ID NO: 140
  Description of Artificial Sequence primer

SEQ ID NO: 181 - DNA, 35 bp, Artificial; identical to SEQ ID NO: 141
  Description of Artificial Sequence primer

SEQ ID NO: 182 - DNA, 35 bp, Artificial; identical to SEQ ID NO: 132
  Description of Artificial Sequence primer

SEQ ID NO: 183 - DNA, 35 bp, Artificial; identical to SEQ ID NO: 133
  Description of Artificial Sequence primer

SEQ ID NO: 184 - DNA, 33 bp, Artificial; identical to SEQ ID NO: 142
  Description of Artificial Sequence primer

SEQ ID NO: 185 - DNA, 33 bp, Artificial; identical to SEQ ID NO: 143
  Description of Artificial Sequence primer

SEQ ID NO: 186 - DNA, 33 bp, Artificial; identical to SEQ ID NO: 134
  Description of Artificial Sequence primer

SEQ ID NO: 187 - DNA, 33 bp, Artificial; identical to SEQ ID NO: 135
  Description of Artificial Sequence primer

SEQ ID NO: 188 - DNA, 35 bp, Artificial; identical to SEQ ID NO: 138
  Description of Artificial Sequence primer

SEQ ID NO: 189 - DNA, 35 bp, Artificial; identical to SEQ ID NO: 139
  Description of Artificial Sequence primer

SEQ ID NO: 190 - DNA, 35 bp, Artificial; identical to SEQ ID NO: 140
  Description of Artificial Sequence primer

SEQ ID NO: 191 - DNA, 35 bp, Artificial; identical to SEQ ID NO: 141
  Description of Artificial Sequence primer

SEQ ID NO: 192 - DNA, 35 bp, Artificial; identical to SEQ ID NO: 132
  Description of Artificial Sequence primer

SEQ ID NO: 193 - DNA, 35 bp, Artificial; identical to SEQ ID NO: 133
  Description of Artificial Sequence primer

SEQ ID NO: 194 - DNA, 32 bp, Artificial
  Description of Artificial Sequence primer
ctccggggagcgctggcgctgtacacagggga

SEQ ID NO: 195 - DNA, 32 bp, Artificial
  Description of Artificial Sequence primer
tcccctgtgtacagcgccagcgctccccggag

SEQ ID NO: 196 - DNA, 31 bp, Artificial
  Description of Artificial Sequence primer
caaggaggccgagaaaatcacgacgggctgt

SEQ ID NO: 197 - DNA, 31 bp, Artificial
  Description of Artificial Sequence primer
acagcccgtcgtgattttctcggcctccttg

SEQ ID NO: 198 - DNA, 37 bp, Artificial
  Description of Artificial Sequence primer
actgcagcttgaatgagaaaatcactgtcccagacac

SEQ ID NO: 199 - DNA, 37 bp, Artificial
  Description of Artificial Sequence primer
gtgtctgggacagtgattttctcattcaagctgcagt

SEQ ID NO: 200 - DNA, 31 bp, Artificial
  Description of Artificial Sequence primer
aggccctgttggtcaaatcttcccagccgtg

SEQ ID NO: 201 - DNA, 31 bp, Artificial
  Description of Artificial Sequence primer
cacggctgggaagatttgaccaacagggcct

SEQ ID NO: 202 - DNA, 33 bp, Artificial
  Description of Artificial Sequence primer
atttcctccggggatggctgaagctgtacacag

SEQ ID NO: 203 - DNA, 33 bp, Artificial
  Description of Artificial Sequence primer
ctgtgtacagcttcagccatccccggaggaaat

SEQ ID NO: 204 - DNA, 35 bp, Artificial
  Description of Artificial Sequence primer
agccgagtcctggaggcggccctcttggaggccaa

SEQ ID NO: 205 - DNA, 35 bp, Artificial
  Description of Artificial Sequence primer
ttggcctccaagagggccgcctccaggactcggct

SEQ ID NO: 206 - DNA, 35 bp, Artificial
  Description of Artificial Sequence primer
agccgagtcctggagagggccctcttggaggccaa

SEQ ID NO: 207 - DNA, 35 bp, Artificial
  Description of Artificial Sequence primer
ttggcctccaagagggccctctccaggactcggct

SEQ ID NO: 208 - DNA, 6059 bp, Artificial
  Description of Artificial Sequence plasmid
ctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggaccatcatcaccatcaccattgat

SEQ ID NO: 209 - DNA, 6059 bp, Artificial
  Description of Artificial Sequence plasmid
ctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtaatgaaacctgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggaccatcatcaccatcaccattgat

SEQ ID NO: 210 - DNA, 6059 bp, Artificial
  Description of Artificial Sequence plasmid
ctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccgacgttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggaccatcatcaccatcaccattgat

SEQ ID NO: 211 - DNA, 6059 bp, Artificial
  Description of Artificial Sequence plasmid
ctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcgagggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggaccatcatcaccatcaccattgat

SEQ ID NO: 212 - DNA, 6059 bp, Artificial
  Description of Artificial Sequence plasmid
ctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccgacgttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcgagggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggaccatcatcaccatcaccattgat

(19) United States
(12) Patent Application Publication
(10) Pub. No.: US 2004/0122216 A1
(43) Pub. Date: Jun. 24, 2004

(54) Recombinant tissue protective cytokines and encoding nucleic acids thereof for protection, restoration, and enhancement of responsive cells, tissues, and organs

(76) Inventors: Jacob Nielsen, Copenhagen (DK); Jan Torleif Pedersen, Bronshoj (DK); Jens Gerwien, Copenhagen (DK); Katrine Bay, Copenhagen (DK); Lars Ostergaard Pedersen, Copenhagen (DK); Marcel Leist, Valby (DK); Marie Aavang Geist, Valby (DK); Pekka Kallunki, Copenhagen (DK); Soren Christensen, Jyllinge (DK); Thomas Sager, Smorum (DK); Michael Brines, Woodbridge, CT (US); Anthony Cerami, Somers, NY (US); Carla Cerami, Sleepy Hollow, NY (US)

Correspondence Address: JONES DAY, 222 EAST 41ST ST, NEW YORK, NY, 10017, US

(21) Appl. No.: 10/612,665

(22) Filed: Jul. 1, 2003

Related U.S. Application Data
(60) Provisional application No. 60/392,455, filed on Jul. 1, 2002.
(60) Provisional application No. 60/393,423, filed on Jul. 3, 2002.

(51) Int. Cl. C07K 14/52

(52) U.S. Cl. 530/351

(57) ABSTRACT

Methods and compositions are provided for protecting or enhancing a responsive cell, tissue, organ or body part function or viability in vivo, in situ or ex vivo in mammals, including human beings, by systemic or local administration of an erythropoietin receptor activity modulator, such as an recombinant tissue protective cytokine.

This application claims priority under 35 U.S.C. § 119(e) to U.S. provisional patent Application No. 60/392,455 filed Jul. 1, 2002, and U.S. provisional patent Application No. 60/393,423 filed Jul. 3, 2002, the entire contents of each of which is incorporated herein by reference in its entirety.

### 1. INTRODUCTION

The present invention is directed to mutein recombinant tissue protective cytokines having one or more amino acid substitutions, pharmaceutical compositions comprising such cytokines for protecting, maintaining, enhancing, or restoring the function or viability of responsive mammalian cells and their associated cells, tissues, and organs. This includes the protection of excitable tissue, such as neuronal and cardiac tissue, from neurotoxins, hypoxia, and other adverse stimuli, and the enhancement of excitable tissue function, such as for facilitating learning and memory. The present invention is further drawn to compositions for transporting or facilitating transport of a molecule via transcytosis across an endothelial cell barrier using mutein recombinant tissue protective cytokines.

### 2. BACKGROUND OF THE INVENTION

For many years, the only clear physiological role of erythropoietin had been its control of the production of red blood cells. Recently, several lines of evidence suggest that erythropoietin, a member of the cytokine superfamily, performs other important physiologic functions which may be mediated through interaction with the erythropoietin receptor (erythropoietin-R). These actions include mitogenesis, modulation of calcium influx into smooth muscle cells and neural cells, vasoactive action, i.e., vasoconstriction/vasodilatation, hyperactivation of platelets and effects on intermediary metabolism. It is believed that erythropoietin provides compensatory responses that serve to improve hypoxic cellular microenvironments as well as modulate programmed cell death caused by metabolic stress. Although studies have established that erythropoietin injected intracranially protects neurons against hypoxic neuronal injury, intracranial administration is an impractical and by and large unacceptable route of administration for therapeutic use, particularly for normal individuals. Furthermore, previous studies of anemic patients given erythropoietin have concluded that peripherally-administered erythropoietin is not transported into the brain (Marti et al., 1997, Kidney Int. 51:416-8; Juul et al., 1999, Pediatr. Res. 46:543-547; Buemi et al., 2000, Nephrol. Dial. Transplant. 15:422-433.).

Various modified forms of erythropoietin have been described with activities directed towards improving the erythropoietic activity of the molecule, such as those having altered amino acids at the carboxy terminus described in U.S. Pat. No. 5,457,089 and in U.S. Pat. No. 4,835,260; erythropoietin isoforms with various numbers of sialic acid residues per molecule, such as those described in U.S. Pat. No. 5,856,298; polypeptides described in U.S. Pat. No. 4,703,008; agonists described in U.S. Pat. No. 5,767,078; peptides which bind to the erythropoietin receptor as described in U.S. Pat. Nos. 5,773,569 and 5,830,851; and small-molecule mimetics as described in U.S. Pat. No. 5,835,382.

It is towards the use of a recombinant tissue protective cytokine for protecting, maintaining, enhancing, or restoring responsive cells and associated cells, tissues, and, organs in situ as well as ex vivo, and to delivery of a recombinant tissue protective cytokine across an endothelial cell barrier for the purpose of protecting and enhancing responsive cells and associated cells, tissues, and organs distal to the vasculature, or to carry associated molecules, that the present invention is directed.

### 3. BRIEF SUMMARY OF THE INVENTION

In one aspect, the present invention is directed to the use of various forms of recombinant tissue protective cytokines for the preparation of pharmaceutical compositions for protecting, maintaining, enhancing, or restoring the function or viability of responsive mammalian cells and their associated cells, tissues, and organs. In one particular aspect, the responsive mammalian cells and their associated cells, tissues, or organs are distal to the vasculature by virtue of a tight endothelial cell barrier. In another particular aspect, the cells, tissues, organs or other bodily parts are isolated from a mammalian body, such as those intended for transplant. By way of non-limiting examples, a responsive cell or tissue may be neuronal, retinal, muscle, heart, lung, liver, kidney, small intestine, adrenal cortex, adrenal medulla, capillary endothelial, testes, ovary, pancreas, bone, skin, or endometrial cells or tissue. Further, non-limiting examples of responsive cells include photoreceptor (rods and cones), ganglion, bipolar, horizontal, amacrine, Müller, Purkinje, myocardium, pace maker, sinoatrial node, sinus node, junction tissue, atrioventricular node, bundle of His, hepatocytes, stellate, Kupffer, mesangial, renal epithelial, tubular interstitial, goblet, intestinal gland (crypts), enteral endocrine, glomerulosa, fasciculate, reticularis, chromaffin, pericyte, Leydig, Sertoli, sperm, Graffian follicle, primordial follicle, islets of Langerhans, α-cells, β-cells, γ-cells, F-cells, osteoprogenitor, osteoclasts, osteoblasts, endometrial stroma, endometrial, stem and endothelial cells. These examples of responsive cells are merely illustrative. In one aspect, the responsive cell or its associated cells, tissues, or organs are not excitable cells, tissues, or organs, nor do they predominantly comprise excitable cells or tissues. In a particular embodiment, the mammalian cell, tissue, or organ for which an aforementioned recombinant tissue protective cytokine is used are those that have expended or will expend a period of time under at least one condition adverse to the viability of the cell, tissue, or organ. In a particular embodiment, the mammalian cell, tissue, or organ for which an aforementioned recombinant tissue protective cytokine is used express the EPO receptor. Such conditions include traumatic in situ hypoxia or metabolic dysfunction, surgically-induced in situ hypoxia or metabolic dysfunction, or in situ toxin exposure, the latter may be associated with chemotherapy or radiation therapy. In one embodiment, the adverse conditions are a result of cardio-pulmonary bypass (heart-lung machine), as is used for certain surgical procedures.

The recombinant tissue protective cytokines of the invention are useful for the therapeutic or prophylactic treatment of human diseases of the central nervous system (CNS) or peripheral nervous system which have primarily neurological or psychiatric symptoms, as well as ophthalmic diseases, cardiovascular diseases, cardiopulmonary diseases, respiratory diseases, kidney, urinary and reproductive diseases, gastrointestinal diseases and endocrine and metabolic abnormalities.

The invention is also directed to pharmaceutical compositions comprising particular aforementioned recombinant tissue protective cytokines for administration to a mammalian animal, preferably a human being. Such pharmaceutical compositions may be formulated for oral, intranasal, or parenteral administration, or in the form of a perfusate solution for maintaining the viability of cells, tissues, or organs ex vivo.

Recombinant tissue protective cytokines useful for the aforementioned purposes may be a mutein, or genetically-modified erythropoietin, that is, an erythropoietin for which at least one modification of the amino acid backbone of the native molecule exists. “Mutant protein,” “variant protein” or “mutein” mean a protein comprising a mutant amino acid sequence and includes polypeptides which differ from the amino acid sequence of native erythropoietin due to amino acid deletions, substitutions, or both. “Native sequence” refers to an amino acid or nucleic acid sequence which is identical to a wild-type or native form of a gene or protein. Furthermore, in one embodiment, the recombinant tissue protective cytokines of the invention have cellular protective activity, but also have one or more of erythropoietin's effects upon the bone marrow, i.e., increased hematocrit (erythropoiesis), vasoactive action (vasoconstriciton/vasodialation), hyperactivation of platelets, increased production of thrombocytes, and pro-coagulant activities. In another embodiment, the recombinant tissue protective cytokines of the invention have cellular protective activity, but does not have one or more of erythropoietin's effects upon the bone marrow, i.e., increased hematocrit (erythropoiesis), vasoactive action (vasoconstriciton/vasodialation), hyperactivation of platelets, increased production of thrombocytes, and pro-coagulant activities. Preferably, a cellular protective recombinant tissue protective cytokine of the invention lacks at least one of erythropoietin's effects on the bone marrow; more preferably the recombinant tissue protective cytokine would lack erythropoietic activity; and most preferably the recombinant tissue protective cytokine lacks all of erythropoietin's effects on the bone marrow.

By way of non-limiting examples, changes in one or more amino acids may be made, or deletions or additions provided, to a native erythropoietin molecule. In a preferred embodiment, the recombinant tissue protective cytokine has one or more modifications in one or more of the following regions: VLQRY (amino acids 11-15 of native, human erythropoietin; SEQ ID NO:1) and/or TKVNFYAW (amino acids 44-51 of native, human erythropoietin; SEQ ID NO:2) and/or SGLRSLTTL (amino acids 100-108 of native, human erythropoietin; SEQ ID NO:3) and/or SNFLRG (amino acids 146-151 of native, human erythropoietin; SEQ ID NO:4). Other mutations may be provided at amino acids 7, 20, 21, 29, 33, 38, 42, 59, 63, 67, 70, 83, 96, 126, 142, 143, 152, 153, 155, 156, and 161 of SEQ ID NO:10. These other mutations may be alone or in addition to at least one mutation in at least one of the regions mentioned above. In certain embodiments, changes in one or more amino acids of TKVNFYAW (amino acids 44-51 of native, human erythropoietin; SEQ ID NO:2) results in a modified erythropoietin molecule with partial function, i.e., having less erythropoietic activity than rhu-EPO. In other embodiments, changes in one or more amino acids of SGLRSLTTL (amino acids 100-108 of native, human erythropoietin; SEQ ID NO:3) results in a recombinant tissue protective cytokine with partial function, i.e., having less erythropoietic activity than rhu-EPO. The above described recombinant tissue protective cytokines exhibit tissue protective or cellular protective activity. With respect to erythropoietic activities, the above described recombinant tissue protective cytokines lack or exhibit a decrease in one or more erythropoietic activities. Examples of erythropoietic activity include increasing hematocrit, vasoconstriction, hyperactivating platelets, pro-coagulant activities and increasing production of thrombocytes. Erythropoietic activities can be measured by techniques standard in the art. For example, hematocrit can be measured using the UT-7 cell assays described in Section 6.17, or using the techniques described in the Physicians' Desk Reference (Medical Economics Company, Inc., Montvale, N.J., 2000,) which is incorporated by reference herein in its entirety. In particular, pages 519-525 and 2125-2131 disclose methods which can be employed in measuring hematocrit levels and different hematocrit ranges are disclosed that can be used as targets to avoid toxicity. For example, in patients with chronic renal failure, the PDR recommends dosing erythropoietin to achieve non-toxic target hematocrits ranging from 30% to 36% in a patient (e.g., see PDR, p. 523, col. 1, 11. 17-96 and p. 2129, col. 1, 11. 8-93, and accompanying table in cols. 2 and 3). The PDR notes that toxicity in the form of polycythemia (a condition marked by an abnormal increase in the number of circulating red blood cells) can be avoided by carefully monitoring the hematocrit and adjusting doses of EPO, withholding erythropoietin if the hematocrit approaches the high-end of the target range (36% for this patient population) or increases by more than 4 points in any 2-week period, until the hematocrit returns to the suggested target range (30% to 36% for this patient population; see PDR, p. 523, col. 1, and p. 2129, col. 1, under “Dose Adjustment”). In contrast, for cancer patients on chemotherapy, the PDR teaches to adjust the dosage at a different hematocrit level, i.e., if the hematocrit exceeds 40% (see p. 2129, col. 2, under “Dose Adjustment”). In one embodiment, the recombinant tissue protective cytokine has one or more erythropoietic activities, but at levels that are not sufficient to cause adverse effects, i.e. effects that outweigh the therapeutic benefit of the cellular protective activity of a recombinant tissue protective cytokine. In one embodiment, the recombinant tissue protective cytokines that possess one or more erythropoietic activities can still be used in the methods of the invention, provided the levels of erythropoietic activity are measured. In those embodiments where the recombinant tissue protective cytokine possesses one or more erythropoietic activities, the erythropoietic activities can be measured and the dose amount and/or dose regimen of the cytokine can be adjusted to ensure the recombinant tissue protective cytokine is not toxic. In those embodiments where the recombinant tissue protective cytokine possesses one or more erythropoietic activities, the erythropoietic activities can be measured and the dose amount and/or dose regimen of the cytokine can be adjusted to ensure the recombinant tissue protective cytokine has low toxicity. In one embodiment, the recombinant tissue protective cytokine exhibits a decrease in one or more erythropoietic activities by about 1%, 2%, 4%, 6%, 8%, 10%,15%, 20%, 25%, 30%, 35%, 40%, 45%, 50%, 55%, 60%, 65%, 70%, 75%, 80%, 85%, 90%, 95%, or 100% in comparison to recombinant Epo.

The invention provides for a recombinant tissue protective cytokine lacking at least one activity selected from the group consisting of increasing hematocrit, vasoconstriction, hyperactivating platelets, pro-coagulant activities and increasing production of thrombocytes. The cytokine comprises at least one responsive cellular protective activity selected from the group consisting of protecting, maintaining, enhancing or restoring the function or viability of a responsive mammalian cell, tissue, or organ.

In one embodiment of the invention, the recombinant tissue protective cytokine comprises one or more altered amino acid residue between position 11 to 15 of SEQ ID NO:10 [SEQ ID NO:1], position 44 to 51 of SEQ ID NO 10 [SEQ ID NO:2], position 100-108 of SEQ ID NO [SEQ ID NO:3], or position 146-151 of SEQ ID NO 10 [SEQ ID NO:4].

In another embodiment, the recombinant tissue protective cytokine comprises an altered amino acid residue at one or more of the following positions of SEQ ID NO:10: 7, 20, 21, 29, 33, 38, 42, 59, 63, 67, 70, 83, 96, 126,142, 143, 152, 153, 155, 156, or 161.

In yet another embodiment, the recombinant tissue protective cytokine comprises the amino acid sequence of SEQ ID NO:10 with one or more of the following changes (each altered sequence has been assigned a separate sequence identification number): an alanine at residue 6 of SEQ ID NO:10 (SEQ ID NO:15); an alanine at residue 7 of SEQ ID NO:10 (SEQ ID NO:16); a serine at residue 7 of SEQ ID NO:10 (SEQ ID NO:17); an isoleucine at residue 10 of SEQ ID NO:10 (SEQ ID NO:18); a serine at residue 11 of SEQ ID NO:10 (SEQ ID NO:19); an alanine at residue 12 of SEQ ID NO:10 (SEQ ID NO:20); an alanine at residue 13 of SEQ ID NO:10 (SEQ ID NO:21); an alanine residue 14 of SEQ ID NO:10 (SEQ ID NO:22); a glutamic acid at residue 14 of SEQ ID NO:10 (SEQ ID NO: 23); a glutamine at residue 14 of SEQ ID NO:10 (SEQ ID NO:24); an alanine at residue 15 of SEQ ID NO:10 (SEQ ID NO:25); a phenylalanine at residue 15 of SEQ ID NO:10 (SEQ ID NO:26); an isoleucine at residue 15 of SEQ ID NO:10 (SEQ ID NO:27); a glutamic acid at residue 20 of SEQ ID NO:10 (SEQ ID NO:28); an alanine at residue 20 of SEQ ID NO:10 (SEQ ID NO:29); an alanine at residue 21 of SEQ ID NO:10 (SEQ ID NO:30); a lysine at residue 24 of SEQ ID NO:10 (SEQ ID NO:31); a serine at residue 29 of SEQ ID NO:10 (SEQ ID NO:32); a tyrosine at residue 29 of SEQ ID NO:10 (SEQ ID NO:33); an asparagine at residue 30 of SEQ ID NO:10 (SEQ ID NO:34); a threonine at residue 32 of SEQ ID NO:10 (SEQ ID NO:35); a serine at residue 33 of SEQ ID NO:10 (SEQ ID NO:36); a tyrosine at residue 33 of SEQ ID NO:10 (SEQ ID NO:37); a lysine at residue 38 of SEQ ID NO:10 (SEQ ID NO:38); a lysine at residue 83 of SEQ ID NO:10 (SEQ ID NO:39); an asparagine at residue 42 of SEQ ID NO:10 (SEQ ID NO:40); an alanine at residue 42 of SEQ ID NO:10 (SEQ ID NO:41); an alanine at residue 43 of SEQ ID NO:10 (SEQ ID NO:42); an isoleucine at residue 44 of SEQ ID NO:10 (SEQ ID NO: 43); an aspartic acid at residue 45 of SEQ ID NO:10 (SEQ ID NO:44); an alanine at residue 45 of SEQ ID NO:10 (SEQ ID NO:45); an alanine at residue 46 of SEQ ID NO:10 (SEQ ID NO:46); an alanine at residue 47 of SEQ ID NO:10 (SEQ ID NO:47); an isoleucine at residue 48 of SEQ ID NO:10 (SEQ ID NO:48); an alanine at residue 48 of SEQ ID NO:10 (SEQ ID NO:49); an alanine at residue 49 of SEQ ID NO:10 (SEQ ID NO:50); a serine at residue 49 of SEQ ID NO:10 (SEQ ID NO:51); a phenylalanine at residue 51 of SEQ ID NO:10 (SEQ ID NO:52); an asparagine at residue 51 of SEQ ID NO:10 (SEQ ID NO:53); an alanine at residue 52 of SEQ ID NO:10 (SEQ ID NO:54); an asparagine at residue 59 of SEQ ID NO:10 (SEQ ID NO:55); a threonine at residue 62 of SEQ ID NO:10 (SEQ ID NO:56); a serine at residue 67 of SEQ ID NO:10 (SEQ ID NO: 57); an alanine at residue 70 of SEQ ID NO:10 (SEQ ID NO:58); an arginine at residue 96 of SEQ ID NO:10 (SEQ ID NO:59); an alanine at residue 97 of SEQ ID NO:10 (SEQ ID NO:60); an arginine at residue 100 of SEQ ID NO:10 (SEQ ID NO:61); a glutamic acid at residue 100 of SEQ ID NO:10 (SEQ ID NO:62); an alanine at residue 100 of SEQ ID NO: 10 (SEQ ID NO:63); a threonine at residue 100 of SEQ ID NO:10 (SEQ ID NO:64); an alanine at residue 101 of SEQ ID NO:10 (SEQ ID NO:65); an isoleucine at residue 101 of SEQ ID NO:10 (SEQ ID NO:66); an alanine at residue 102 of SEQ ID NO:10 (SEQ ID NO:67); an alanine at residue 103 of SEQ ID NO:10 (SEQ ID NO:68); a glutamic acid at residue 103 of SEQ ID NO:10 (SEQ ID NO:69); an alanine at residue 104 of SEQ ID NO: 10 (SEQ ID NO:70); an isoleucine at residue 104 of SEQ ID NO:10 (SEQ ID NO:71); an alanine at residue 105 of SEQ ID NO:10 (SEQ ID NO:72); an alanine at residue 106 of SEQ ID NO:10 (SEQ ID NO:73); an isoleucine at residue 106 of SEQ ID NO:10 (SEQ ID NO:74); an alanine at residue 107 of SEQ ID NO:10 (SEQ ID NO:75); a leucine at residue 107 of SEQ ID NO:10 (SEQ ID NO:76); a lysine at residue 108 of SEQ ID NO:10 (SEQ ID NO:77); an alanine at residue 108 of SEQ ID NO:10 (SEQ ID NO:78); a serine at residue 108 of SEQ ID NO:10 (SEQ ID NO:79); an alanine at residue 116 of SEQ ID NO:10 (SEQ ID NO:80); an alanine at residue 126 of SEQ ID NO:10 (SEQ ID NO:81); an alanine at residue 132 of SEQ ID NO:10 (SEQ ID NO:82); an alanine at residue 133 of SEQ ID NO:10 (SEQ ID NO:83); an alanine at residue 134 of SEQ ID NO:10 (SEQ ID NO:84); an alanine at residue 140 of SEQ ID NO:10 (SEQ ID NO:85); an isoleucine at residue 142 of SEQ ID NO:10 (SEQ ID NO:86); an alanine at residue 143 of SEQ ID NO: 10 (SEQ ID NO:87); an alanine at residue 146 of SEQ ID NO:10 (SEQ ID NO:88); a lysine at residue 147 of SEQ ID NO:10 (SEQ ID NO:89); an alanine at residue 147 of SEQ ID NO:10 (SEQ ID NO:90); a tyrosine at residue 148 of SEQ ID NO:10 (SEQ ID NO: 91); an alanine at residue 148 of SEQ ID NO:10 (SEQ ID NO:92); an alanine at residue 149 of SEQ ID NO:10 (SEQ ID NO:93); an alanine at residue 150 of SEQ ID NO:10 (SEQ ID NO:94); a glutamic acid at residue 150 of SEQ ID NO:10 (SEQ ID NO:95); an alanine at residue 151 of SEQ ID NO:10 (SEQ ID NO:96); an alanine at residue 152 of SEQ ID NO:10 (SEQ ID NO:97); a tryptophan at residue 152 of SEQ ID NO:10 (SEQ ID NO:98); an alanine at residue 153 of SEQ ID NO:10 (SEQ ID NO:99); an alanine at residue 154 of SEQ ID NO:10 (SEQ ID NO:100); an alanine at residue 155 of SEQ ID NO:10 (SEQ ID NO:101); an alanine at residue 158 of SEQ ID NO:10 (SEQ ID NO: 102); a serine at residue 160 of SEQ ID NO:10 (SEQ ID NO:103); an alanine at residue 161 of SEQ ID NO:10 (SEQ ID NO:104); or an alanine at residue 162 of SEQ ID NO:10 (SEQ ID NO:105). In one embodiment, the recombinant tissue protective cytokine comprises the amino acid sequence of SEQ ID NO:10 with one or more of the amino acid residue substitutions of SEQ ID NOs: 15-105 and 119.

In yet another embodiment, the recombinant tissue protective cytokine comprises the amino acid sequence of SEQ ID NO:10 with a deletion of amino acid residues 44-49 of SEQ ID NO:10.

In still another embodiment, the recombinant tissue protective cytokine comprises, the amino acid sequence of SEQ ID NO:10 with at least one of the following changes (each altered sequence has been assigned a separate sequence identification number): i) an aspartic acid at residue 45, and a glutamic acid at residue 100 of SEQ ID NO:10 (SEQ ID NO:106); ii) an asparagine at residue 30, a threonine at residue 32 of SEQ ID NO:10 (SEQ ID NO:107); iii) an aspartic acid at residue 45, a glutamic acid at residue 150 SEQ ID NO:10 (SEQ ID NO:108); iv) a glutamic acid at residue 103, and a serine at residue 108 of SEQ ID NO:10 (SEQ ID NO:109); v) an alanine at residue 140 and an alanine at residue 52 of SEQ ID NO:10 (SEQ ID NO:110); vi) an alanine at residue 140, an alanine at residue 52, an alanine at residue 45 of SEQ ID NO:10 (SEQ ID NO:111); vii) an alanine at residue 97, and an alanine at residue 152 of SEQ ID NO:10 (SEQ ID NO:112); iix) an alanine at residue 97, an alanine at residue 152, an alanine at residue 45 of SEQ ID NO:10 (SEQ ID NO:113); ix) an alanine at residue 97, an alanine at residue 152, an alanine at residue 45, and an alanine at residue 52 of SEQ ID NO:10 (SEQ ID NO:114); x) an alanine at residue 97, an alanine at residue 152, an alanine at residue 45, an alanine at residue 52, and an alanine at residue 140 of SEQ ID NO:10 (SEQ ID NO:115); xi) an alanine at residue 97, an alanine at residue 152, an alanine at residue 45, an alanine at residue 52, an alanine at residue 140, an alanine at residue 154, a lysine at residue 24, a lysine at residue 38, a lysine at residue 83, a lysine at residue 24 and an alanine at residue 15 of SEQ ID NO:10 (SEQ ID NO:116); xii) a lysine at residue 24, a lysine at residue 38, and a lysine at residue 83 SEQ ID NO:10 (SEQ ID NO:117); or xiv) a lysine at residue 24 and an alanine at residue 15 SEQ ID NO:10 (SEQ ID NO:118). In one embodiment, the recombinant tissue protective cytokine comprises, the amino acid sequence of SEQ ID NO: 10 with at least one of the following amino acid residue substitutions of SEQ ID NOs: 106-118.

One embodiment of the invention is directed to the recombinant tissue protective cytokine as described herein above, further comprising a chemical modification of one or more amino acids. In another embodiment the chemical modification comprises altering the charge of the recombinant tissue protective cytokine. In yet another embodiment, a positive or negative charge is chemically added to an amino acid residue where a charged amino acid residue is modified to an uncharged residue.

Moreover, such aforementioned recombinant tissue protective cytokines may be further modified by having a chemical modification of one or more amino acids, such as described in the following co-pending applications: PCT application serial no. PCT/US01/49479, filed Dec. 28, 2001, U.S. patent application Ser. No.09/753,132 filed Dec. 29, 2000, and U.S. Patent Application Attorney Docket No. KW00-009C02-US filed Jul. 3, 2002, each of these applications is incorporated herein by reference in their entirety. These further chemical modifications may be used to enhance the tissue protective activities of the recombinant tissue protective cytokines or suppress any effects the recombinant tissue protective cytokines may have on bone marrow. In a further embodiment, the additional chemical modification is provided to restore solubility of the molecule that may be reduced as a result of the aforementioned genetic modification, such as chemically adding a positive or negative charge to the molecule if a charged amino acid residue is changed to an uncharged residue.

By way of non-limiting examples, recombinant tissue protective cytokines of the invention include human erythropoietin mutein S100E (SEQ ID NO:5), human erythropoietin mutein K45D (SEQ ID NO:6), and any of the nonerythropoietic yet cellular protective recombinant tissue protective cytokines or those able to benefit a responsive cell, tissue or organ, that are described in Elliott et al., 1997, Blood 89:493-502; Boissel et al., Journal of Biological Chemistry, vol. 268, No. 21, pp. 15983-15993 (1993); Wen et al., Journal of Biological Chemistry, vol. 269, No. 36, pp. 22839-22846 (1994); and Syed et al., Nature, vol. 395, pp. 511-516 (1998), which are incorporated herein by reference in their entireties. The present invention is directed to methods for the use of any of the aforementioned recombinant tissue protective cytokines for the protection, restoration, and enhancement of responsive cells, tissues, and organs.

Other recombinant tissue protective cytokines of the invention include an aforementioned erythropoietin comprising at least one genetically altered amino acid with at least one additional modification which may be another modification of at least one additional amino acid of the erythropoietin molecule, or a modification of at least one carbohydrate of the erythropoietin molecule. The genetically altered amino acid(s) may be the one or among those further modified. Of course, recombinant tissue protective cytokine molecules useful for the purposes herein may have a plurality of modifications as compared to the native erythropoietin molecule, such as multiple modifications of the amino acid portion of the molecule, multiple modifications of the carbohydrate portion of the molecule, or at least a second modification of the amino acid portion of the molecule and at least one modification of the carbohydrate portion of the molecule. The recombinant tissue protective cytokine molecule retains its ability of protecting, maintaining, enhancing or restoring the function or viability of responsive mammalian cells, yet other properties of the recombinant tissue protective cytokine unrelated to the aforementioned, desirable feature may be absent as compared to the native molecule. In a preferred embodiment, the recombinant tissue protective cytokine is non-erythropoietic.

In another embodiment, the recombinant tissue protective cytokines can be modified by fucosylation to alter glycoslyation patterns on a glycoprotein.

One embodiment of the invention is directed to the recombinant tissue protective cytokine as described herein above is a human erythropoietin mutein. In another embodiment of the invention the recombinant tissue protective cytokine is a human phenylglyoxal erythropoietin mutein. In another embodiment of the invention, the recombinant tissue protective cytokine is a human asialoerythropoietin mutein.

In one embodiment, as described herein above, the recombinant tissue protective cytokine comprises at least one responsive cellular protective activity selected from the group consisting of protecting, maintaining, enhancing or restoring the function or viability of a responsive mammalian cell, tissue, or organ. In such an embodiment, the responsive mammalian cell comprises a neuronal, muscle, heart, lung, liver, kidney, small intestine, adrenal cortex, adrenal medulla, capillary, endothelial, testes, ovary, endometrial, or stem cell. In other embodiments, the cell comprises a photoreceptor, ganglion, bipolar, horizontal, amacrine, Müller, myocardium, pace maker, sinoatrial node, sinus node, atrioventricular node, bundle of His, hepatocyte, stellate, Kupffer, mesangial, goblet, intestinal gland, enteral endocrine, glomerulosa, fasciculate, reticularis, chromaffin, pericyte, Leydig, Sertoli, sperm, Graffian follicles, primordial follicles, endometrial stroma cells, or endometrial cell.

According to another aspect of the invention, the recombinant tissue protective cytokine, as described herein above, is capable of traversing an endothelial cell barrier. In a related embodiment, the endothelial cell barrier comprises the blood-brain barrier, the blood-eye barrier, the blood testis barrier, the blood-ovary barrier, blood-placenta, blood-heart, blood-kidney, and the blood-uterus barrier.

In another embodiment of the invention, the recombinant tissue protective cytokine as described herein above is further modified. In one embodiment, the recombinant tissue protective cytokine is selected from the group consisting of: i) a cytokine having a reduced number or no sialic acid moieties; ii) a cytokine having a reduced number or no N-linked or O-linked carbohydrates; iii) a cytokine having at least a reduced carbohydrate content by virtue of treatment of native cytokine with at least one glycosidase; iv) a cytokine having at least one or more oxidized carbohydrates; v) a cytokine having at least one or more oxidized carbohydrates and is chemically reduced; vi) a cytokine having at least one or more modified arginine residues; vii) a cytokine having at least one or more modified lysine residues or a modification of the N-terminal amino group of a cytokine molecule; viii) a cytokine having at least a modified tyrosine residue; ix) a cytokine having at least a modified aspartic acid or glutamic acid residue; x) a cytokine having at a modified tryptophan residue; xi) a cytokine having at least one amino acid group removed; xii) a cytokine having at least one opening of at least one of the cystine linkages in the cytokine molecule; xiii) a truncated cytokine; xiv) a cytokine having at least one polyethylene glycol molecule attached; xv) a cytokine having at least one fatty acid attached; xvi) a cytokine having a non-mammalian glycosylation pattern by virtue of the expression of a recombinant cytokine in non-mammalian cells; and xvi) a cytokine having at least one histidine tagged amino acid to facilitate purification.

In one embodiment, the recombinant tissue protective cytokine of the invention has a reduced number of sialic acid moieties, or no sialic acid moieties. In a preferred embodiment, the recombinant tissue protective cytokine is the asialo form of an erythropoietin (i.e. has no sialic acid moieties), and most preferably, a human asialoerythropoietin. In another embodiment, the recombinant tissue protective cytokine has 1, 2, 3, 4, 5, 6, 7, 8, 9, 10, 11, 12, or 13 sialic acid moieties. The number of available sites for sialylation may be altered by the presence of one or more altered or modified amino acids in the recombinant tissue protective cytokine. Therefore, the present invention covers embodiments wherein the recombinant tissue protective cytokine is either hyposialylated or hypersialylated. In a preferred aspect, the erythropoietin mutein has more than the 14 sialic acid moieties present in native erythropoietin.

In one embodiment, the recombinant tissue protective cytokine is an erythropoietin with no N-linked carbohydrates. In another embodiment, the recombinant tissue protective cytokine is an erythropoietin with a reduced number of N-linked carbohydrates. In one embodiment, the recombinant tissue protective cytokine is an erythropoietin with no O-linked carbohydrates. In another embodiment, the recombinant tissue protective cytokine is an erythropoietin with a reduced number of O-linked carbohydrates.

In another embodiment, the recombinant tissue protective cytokines can be modified by fucosylation to alter glycoslyation patterns on a glycoprotein.

In yet another embodiment, the recombinant tissue protective cytokine is treated with at least one glycosidase. In another embodiment, the recombinant tissue protective cytokine has at least a reduced carbohydrate content by virtue of treatment of the recombinant tissue protective cytokine with at least one glycosidase.

In yet another embodiment, the carbohydrate portion of the recombinant tissue protective cytokine has at least a non-mammalian glycosylation pattern by virtue of the expression of a recombinant erythropoietin in non-mammalian cells. In preferred embodiments, the recombinant tissue protective cytokines are expressed in insect cells, plant cells, bacteria cells, or yeast cells.

In yet another embodiment, the recombinant tissue protective cytokine further has at least one or more oxidized carbohydrates which also may be chemically reduced. In a preferred embodiment, the recombinant tissue protective cytokine is periodate-oxidized erythropoietin. In certain embodiments, the periodate-oxidized erythropoietin is chemically reduced with sodium cyanoborohydride.

In yet another embodiment, the recombinant tissue protective cytokine for the aforementioned uses has at least one or more modified arginine residues. In one embodiment, the recombinant tissue protective cytokine comprises an R-glyoxal moiety on the one or more arginine residues, wherein R is aryl or alkyl moiety. In yet another embodiment, the recombinant tissue protective cytokine is phenylglyoxal-erythropoietin. In yet another embodiment, the recombinant tissue protective cytokine is an erythropoietin in which an arginine residue is modified by reaction with a vicinal diketone, such as but not limited to, 2,3-butanedione and cyclohexanedione. In yet another embodiment, the recombinant tissue protective cytokine is an erythropoietin in which an arginine residue is reacted with 3-deoxyglucosone.

In yet another embodiment, the recombinant tissue protective cytokine comprises at least one or more modified lysine residues or a modification of the N-terminal amino group of the erythropoietin molecule, such modifications as those resulting from reaction of the lysine residue or N-terminal amino group with an amino-group-modifying agent. The modified lysine residue further may be chemically reduced. In one preferred embodiment, a recombinant tissue protective cytokine is biotinylated or carbamylated or acylated, such as acetylated, via one or more lysine groups. In another preferred embodiment, the lysine is reacted with an aldehyde or reducing sugar to form an imine, which may be stabilized by reduction as with sodium cyanoborohydride to form an N-alkylated lysine such as glucitolyl lysine, or which in the case of reducing sugars may be stabilized by Amadori or Heyns rearrangement to form an alpha-deoxy alpha-amino sugar such as alpha-deoxy-alpha-fructosyllysine. In another preferred embodiment, the lysine group is carbamylated (carbamoylated), such as by virtue of reaction with cyanate ion, alkyl-carbamylated, aryl-carbamylated, or aryl-thiocarbamylated with an alkyl-isocyanate, aryl-isocyanate, or aryl isothiocyanate, respectively, or it may be acylated by a reactive alkylcarboxylic or arylcarboxylic acid derivative, such as by reaction with acetic anhydride, succinic anhydride or phthalic anhydride. At least one lysine group may also be trinitrophenyl, modified by reaction with a trinitrobenzenesulfonic acid, or preferably its salts. In another embodiment, lysine residues may be modified by reaction with a glyoxal derivative, such as reaction with glyoxal, methylglyoxal or 3-deoxyglucosone, to form the corresponding alpha-carboxyalkyl derivatives. In a related embodiment, the carbamylated cytokine is comprised of alpha-N-carbamoylerythropoietin; N-epsilon-carbamoylerythropoietin; alpha-N-carbamoyl, N-epsilon-carbamoylerythropoietin; alpha-N-carbamoylasialoerythropoietin; N-epsilon-carbamoylasialoerythropoietin; alpha-N-carbamoyl, N-epsilon-carbamoylasialoerythropoietin; alpha-N-carbamoylhyposialoerythropoietin; N-epsilon-carbamoylhyposialoerythropoietin; and alpha-N-carbamoyl, N-epsilon-carbamoylhyposialoerythropoietin. In yet another embodiment, the recombinant tissue protective cytokine comprises at least one acylated lysine residue. In yet another embodiment, the recombinant tissue protective cytokine comprises at least one acylated lysine residue. In yet another embodiment, the recombinant tissue protective cytokine comprises at least one acylated lysine residue. In a related embodiment, the acetylated cytokine is comprised of alpha-N-acetylerythropoietin; N-epsilon-acetylerythropoietin; alpha-N-acetyl, N-epsilon-acetylerythropoietin; alpha-N-acetylasialoerythropoietin; N-epsilon-acetylasialoerythropoietin; alpha-N-acetyl, N-epsilon-acetylasialoerythropoietin; alpha-N-acetylhyposialoerythropoietin; N-epsilon-acetylhyposialoerythropoietin; alpha-N-acetyl, N-epsilon-acetylhyposialoerythropoietin; alpha-N-acetylhypersialoerythropoietin; N-epsilon-acetylhypersialoerythropoietin; alpha-N-acetyl, and N-epsilon-acetylhypersialoerythropoietin.

In yet another embodiment, the recombinant tissue protective cytokine has a lysine residue that is succinylated. In a related embodiment, the succinylated cytokine is comprised of alpha-N-succinylerythropoietin; N-epsilon-succinylerythropoietin; alpha-N-succinyl, N-epsilon-succinylerythropoietin; alpha-N-succinylasialoerythropoietin; N-epsilon-succinylasialoerythropoietin; alpha-N-succinyl, N-epsilon-succinylasialoerythropoietin; alpha-N-succinylhyposialoerythropoietin; N-epsilon-succinylhyposialoerythropoietin; alpha-N-succinyl, N-epsilon-succinylhyposialoerythropoietin; alpha-N-succinylhypersialoerythropoietin; N-epsilon-succinylhypersialoerythropoietin; and N-epsilon-succinylhypersialoerythropoietin.

In one embodiment, at least one tyrosine residue of a recombinant tissue protective cytokine may be modified in an aromatic ring position by an electrophilic reagent, such as by nitration or iodination. In a related embodiment, the recombinant tissue protective cytokine as described herein above comprises at least one lysine residue modified by 2, 4, 6 trintrobenzenesulfonate sodium or another salt thereof.

In another embodiment, the recombinant tissue protective cytokine comprises at least one nitrated or iodinated tyrosine residue.

In another embodiment, the recombinant tissue protective cytokine comprises an aspartic acid or glutamic acid residue that is reacted with a carbodiimide followed by reaction with an amine. In a related embodiment, the amine is glycinamide.

In one embodiment, at least a tryptophan residue of a recombinant tissue protective cytokine is modified, such as by reaction with n-bromosuccinimide or n-chlorosuccinimide.

In another embodiment, a recombinant tissue protective cytokine is provided having at least one erythropoietin amino group removed, such as by reaction with ninhydrin followed by reduction of the resulting carbonyl group by reaction with borohydride.

In yet another embodiment, a recombinant tissue protective cytokine is provided having at least an opening of at least one of the cystine linkages in the molecule by reaction with a reducing agent such as dithiothreitol, followed by reaction of the subsequent sulfhydryls with iodoacetamide, iodoacetic acid or another electrophile to prevent reformation of the disulfide linkages.

In yet another embodiment, a recombinant tissue protective cytokine is subjected to a limited chemical proteolysis that targets specific residues, for example, to cleave after tryptophan residues. Such resulting recombinant tissue protective cytokine fragments are embraced herein.

As noted above, a recombinant tissue protective cytokine useful for the purposes herein optionally may have at least one of the aforementioned chemical modifications in addition to the genetically altered amino acid(s), but may have more than one of the above modifications. By way of example of a recombinant tissue protective cytokine with one modification to the carbohydrate portion of the molecule and one modification to the amino acid portion, a recombinant tissue protective cytokine is an asialoerythropoietin that has its lysine residues biotinylated, acylated (such as acetylated) or carbamylated. The recombinant tissue protective cytokines can also be modified by the addition of fatty acid chains. In another embodiment, a recombinant tissue protective cytokine is modified by pegalation, to create pegylated tissue protective cytokines by the addition of polyethylene glycol (PEG).

According to one aspect of the invention, there is provided an isolated nucleic acid molecule that comprises a nucleotide sequence which encodes a polypeptide comprising the recombinant tissue protective cytokine as described herein above. In one embodiment, the isolated nucleic acid molecule comprises the nucleotide sequence of nucleotide residues 5461 through 6041 of the vecotor contruct of SEQ ID NO:208, nucleotide residues 5461 through 6041 of SEQ ID NO:209, nucleotide residues 5461 through 6041 of SEQ ID NO: 210, nucleotide residues 5461 through 6041 of SEQ ID NO:211, or nucleotide residues 5461 through 6041 of SEQ ID NO:212.

In one embodiment of the invention, there is provided an isolated nucleic acid molecule that comprises a nucleotide sequence (i.e., a cDNA, a nucleotide sequence interrupted by introns, or uninterrupted by introns), which encodes a polypeptide comprising or consisting of the recombinant tissue protective cytokine as described herein above with the proviso that the nucleic acid molecule does not encode a recombinant tissue protective cytokine that comprises one or more of the following amino acid substitutions: I16A, C7A, K20A, P42A, D43A, K45D, K45A, F48A, Y49A, K52A, K49A, S100E, R103A, K116A, T132A, I133A, K140A, N147K, N147A, R150A, R150E, G151A, K152A, K154A, G158A, C161A, or R162A. In a related embodiment, there is provided an isolated nucleic acid molecule that comprises a nucleotide sequence which encodes a polypeptide comprising the recombinant tissue protective cytokine as described herein above with the proviso that the nucleic acid molecule does not encode a recombinant tissue protective cytokine that comprises any of the following combinations of substitutions: N24K/N38K/N83K or A30N/H32T. In one embodiment, the a nucleotide sequence, encoding the recombinant tissue protective cytokine, is synthesized using preferred codons that facilitate optimal expression in a particular host cell. Such preferred codons can be optimal for expression in cells of a species of plant, bacteria, yeast, mammal, fungi, or insect.

The invention also provides for a vector comprising the nucleic acid molecule. The invention also provides for an expression vector comprising the nucleic acid molecule and at least one regulatory region operably linked to the nucleic acid molecule. In one embodiment, the vector is a pCiNeo vector. In another embodiment, the invention provides for a cell comprising the expression vector. In yet another embodiment, there is provided a genetically-engineered cell which comprises the nucleic acid molecule.

In another embodiment, the present invention also embraces compositions, including pharmaceutical compositions, comprising one or more of the aforementioned recombinant tissue protective cytokines.

According to another aspect of the invention, there is provided a pharmaceutical composition comprising a recombinant tissue protective cytokine as described herein above, lacking at least one erythropoietic activity selected from the group consisting of increasing hematocrit, vasoconstriction, hyperactivating platelets, pro-coagulant activities and increasing production of thrombocytes. According to another aspect of the invention, there is provided a pharmaceutical composition comprising a recombinant tissue protective cytokine as described herein above, but the cytokines do not lack at least one erythropoietic activity selected from the group consisting of increasing hematocrit, vasoconstriction, hyperactivating platelets, pro-coagulant activities and increasing production of thrombocytes. The cytokine comprises at least one responsive cellular protective activity selected from the group consisting of protecting, maintaining, enhancing or restoring the function or viability of a responsive mammalian cell, tissue or organ. The recombinant tissue protective cytokine of the pharmaceutical composition may comprise the amino acid sequence of SEQ ID NO:10 with at least one of the following changes, i.e. substitutions, (each change or combination of changes listed has been assigned a separate sequence identification number): i) an aspartic acid at residue 45, and a glutamic acid at residue 100 of SEQ ID NO:10 (SEQ ID NO:106); ii) an asparagine at residue 30, a threonine at residue 32 of SEQ ID NO:10 (SEQ ID NO:107); iii) an aspartic acid at residue 45, a glutamic acid at residue 150 SEQ ID NO:10 (SEQ ID NO:108); iv) a glutamic acid at residue 103, and a serine at residue 108 of SEQ ID NO:10 (SEQ ID NO:109); v) an alanine at residue 140 and an alanine at residue 52 of SEQ ID NO:10 (SEQ ID NO:110); vi) an alanine at residue 140, an alanine at residue 52, an alanine at residue 45 of SEQ ID NO:10 (SEQ ID NO: 111); vii) an alanine at residue 97, and an alanine at residue 152 of SEQ ID NO:10 (SEQ ID NO:112); iix) an alanine at residue 97, an alanine at residue 152, an alanine at residue 45 of SEQ ID NO:10 (SEQ ID NO:113); ix) an alanine at residue 97, an alanine at residue 152, an alanine at residue 45, and an alanine at residue 52 of SEQ ID NO:10 (SEQ ID NO: 114); x) an alanine at residue 97, an alanine at residue 152, an alanine at residue 45, an alanine at residue 52, and an alanine at residue 140 of SEQ ID NO:10 (SEQ ID NO:115); xi) an alanine at residue 97, an alanine at residue 152, an alanine at residue 45, an alanine at residue 52, an alanine at residue 140, an alanine at residue 154, a lysine at residue 24, a lysine at residue 38, a lysine at residue 83, a lysine at residue 24 and an alanine at residue 15 of SEQ ID NO:10 (SEQ ID NO:116); xii) a lysine at residue 24, a lysine at residue 38, and a lysine at residue 83 SEQ ID NO:10 (SEQ ID NO:117); or xiv) a lysine at residue 24 and an alanine at residue 15 SEQ ID NO:10 (SEQ ID NO:118).

According to another aspect of the invention, there is provided a pharmaceutical composition for protecting, maintaining, enhancing, or restoring the function or viability of responsive mammalian cells and their associated cells, tissues, and organs, comprising a therapeutically effective amount of a recombinant tissue protective cytokine, comprising at least one of the following amino acid residue substitutions: (each change or combination of changes listed has been assigned a separate sequence identification number): a tryptophan at residue 152 of SEQ ID NO:10 (SEQ ID NO:98); an alanine at residue 14 and an alanine at residue 15 of SEQ ID NO:10 (SEQ ID NO:119); an alanine at residue 6 of SEQ ID NO:10 (SEQ ID NO:15); an alanine at residue 7 of SEQ ID NO:10 (SEQ ID NO:16); an alanine at residue 43 of SEQ ID NO:10 (SEQ ID NO:42); an alanine at residue 42 of SEQ ID NO: 10 (SEQ ID NO:41); an alanine at residue 48 of SEQ ID NO:10 (SEQ ID NO:49); an alanine at residue 49 of SEQ ID NO:10 (SEQ ID NO:50); an threonine at residue 32 of SEQ ID NO:10 (SEQ ID NO:35); an alanine at residue 133 of SEQ ID NO:10 (SEQ ID NO:83); an alanine at residue 134 of SEQ ID NO:10 (SEQ ID NO:84); an alanine at residue 147 of SEQ ID NO:10 (SEQ ID NO:90); an alanine at residue 148 of SEQ ID NO: 10 (SEQ ID NO:92); an alanine at residue 150 of SEQ ID NO:10 (SEQ ID NO:94); an alanine at residue 151 of SEQ ID NO:10 (SEQ ID NO:96); an alanine at residue 158 of SEQ ID NO:10 (SEQ ID NO:102); an alanine at residue 161 of SEQ ID NO:10 (SEQ ID NO:104); or an alanine at residue 162 of SEQ ID NO:10 (SEQ ID NO:105).

In one embodiment, the pharmaceutical composition described above herein is formulated for oral, intranasal, or parenteral administration. In another embodiment, the pharmaceutical composition is formulated as a perfusate solution.

In certain embodiments, the pharmaceutical compositions of the invention for protecting, maintaining, enhancing, or restoring the function or viability of responsive mammalian cells and their associated cells, tissues, and organs, comprise a therapeutically effective amount of a recombinant tissue protective cytokine, comprising at least one substitution of amino acid residues of native, human erythropoietin amino acid sequence.

In other embodiments, a pharmaceutical composition of the invention for protecting, maintaining, enhancing, or restoring the function or viability of responsive mammalian cells and their associated cells, tissues, and organs, comprises a therapeutically effective amount of a recombinant tissue protective cytokine, comprising cellular protective activity may lack one or more erythropoietic activities or effects such as increasing hematocrit, vasoactive action (vasoconstriction/vasodilatation), hyperactivating platelets, pro-coagulant activities and increasing production of thrombocytes.

In other embodiments, a pharmaceutical composition of the invention for protecting, maintaining, enhancing, or restoring the function or viability of responsive mammalian cells and their associated cells, tissues, and organs, comprises a therapeutically effective amount of a recombinant tissue protective cytokine, comprising cellular protective activity also has one or more erythropoietic activities or effects such as increasing hematocrit, vasoactive action (vasoconstriction/vasodilatation), hyperactivating platelets, pro-coagulant activities and increasing production of thrombocytes.

According to one aspect of the invention, there is provided a method for protecting, maintaining or enhancing the viability of a cell, tissue, or organ isolated from a mammalian body comprising exposing said cell, tissue, or organ to a pharmaceutical composition comprising a recombinant tissue protective cytokine comprised of an erythropoietin that lacks at least one erythropoietic activity selected from the group consisting of increasing hematocrit, vasoactive action (vasoconstriction/vasodilatation), hyperactivating platelets, pro-coagulant activity and increasing production of thrombocytes. In certain embodiments, the protection does not effect bone marrow.

The invention also provides for a method for protecting, maintaining or enhancing the viability of a cell, tissue, or organ isolated from a mammalian body comprising exposing said cell, tissue, or organ to a pharmaceutical composition comprising a recombinant tissue protective cytokine comprised, as described herein above, that lacks at least one erythropoietic activity selected from the group consisting of increasing hematocrit, vasoactive action (vasoconstriction/vasodilatation), hyperactivating platelets, pro-coagulant activity and increasing production of thrombocytes.

The invention further provides for the use of a recombinant tissue protective cytokine as described herein above, that lacks at least one erythropoietic activity selected from the group consisting of increasing hematocrit, vasoactive action (vasoconstriction/vasodilatation), hyperactivating platelets, pro-coagulant activity and increasing production of thrombocytes, for the preparation of a pharmaceutical composition for the protection against and prevention of a tissue injury as well as the restoration of and rejuvenation of tissue and tissue function in a mammal. In one embodiment, the injury is caused by a seizure disorder, multiple sclerosis, stroke, hypotension, cardiac arrest, ischemia, myocardial infarction, inflammation, age-related loss of cognitive function, radiation damage, cerebral palsy, neurodegenerative disease, Alzheimer's disease, Parkinson's disease, Leigh's disease, AIDS dementia, memory loss, amyotrophic lateral sclerosis, alcoholism, mood disorder, anxiety disorder, attention deficit disorder, hyperactivity, autism, Creutzfeld-Jakob disease, brain or spinal cord trauma or ischemia, heart-lung bypass, chronic heart failure, macular degeneration, diabetic neuropathy, diabetic retinopathy, glaucoma, retinal ischemia, or retinal trauma.

According to another aspect of the invention, there is provided a method for facilitating the transcytosis of a molecule across an endothelial cell barrier in a mammal comprising administration to said mammal a composition comprising said molecule in association with a recombinant tissue protective cytokine as described herein above, lacking at least one activity selected from the group consisting of increasing hematocrit, increasing blood pressure, hyperactivating platelets, and increasing production of thrombocytes. In one embodiment, the association is a labile covalent bond, a stable covalent bond, or a non-covalent association with a binding site for said molecule. According to another aspect of the invention, there is provided a method for facilitating the transcytosis of a molecule across an endothelial cell barrier in a mammal comprising administration to said mammal a composition comprising said molecule in association with a recombinant tissue protective cytokine as described herein above, and having activity selected from the group consisting of increasing hematocrit, increasing blood pressure, hyperactivating platelets, and increasing production of thrombocytes. In one embodiment, the association is a labile covalent bond, a stable covalent bond, or a non-covalent association with a binding site for said molecule. In another embodiment, the endothelial cell barrier is selected from the group consisting of the blood-brain barrier, the blood-eye barrier, the blood-testis barrier, the blood-ovary barrier, the blood-heart, the blood kidney, and the blood-placenta barrier. In yet another embodiment, the molecule is a receptor agonist or antagonist hormone, a neurotrophic factor, an antimicrobial agent, an antiviral agent, a radiopharmaceutical, an antisense oligonucleotide, an antibody, an immunosuppressant, a dye, a marker, or an anti-cancer drug.

According to another aspect of the invention, there is provided a composition for transporting a molecule via transcytosis across an endothelial cell barrier comprising said molecule in association with a recombinant tissue protective cytokine, as described herein above, lacking at least one erythropoietic activity selected from the group consisting of increasing hematocrit, vasoactive action (vasoconstriction/vasodilatation), hyperactivating platelets, pro-coagulant activity and increasing production of thrombocytes. According to another aspect of the invention, there is provided a composition for transporting a molecule via transcytosis across an endothelial cell barrier comprising said molecule in association with a recombinant tissue protective cytokine, as described herein above, and having at least one erythropoietic activity selected from the group consisting of increasing hematocrit, vasoactive action (vasoconstriction/vasodilatation), hyperactivating platelets, pro-coagulant activity and increasing production of thrombocytes. In one embodiment, the association is a labile covalent bond, a stable covalent bond, or a non-covalent association with a binding site for said molecule. In another embodiment, the molecule is a receptor agonist or antagonist hormone, a neurotrophic factor, an antimicrobial agent, a radiopharmaceutical, an antisense oligonucleotide, an antibody, an immunosuppressant, a dye, a marker, or an anti-cancer drug.

The invention also provides for the use of an recombinant tissue protective cytokine as described herein above, lacking at least one erythropoietic activity selected from the group consisting of increasing hematocrit, vasoactive action (vasoconstriction/vasodilatation), hyperactivating platelets, pro-coagulant activities and increasing production of thrombocytes. In one embodiment, the association is a labile covalent bond, a stable covalent bond, or a non-covalent association with a binding site for said molecule. In another embodiment, the molecule is a receptor agonist or antagonist hormone, a neurotrophic factor, an antimicrobial agent, a radiopharmaceutical, an antisense oligonucleotide, an antibody, an immunosuppressant, a dye, or a marker, or an anti-cancer drug.

Thus, the invention is directed to a cellular protective use of any recombinant tissue protective cytokine with an alteration in at least one amino acid of the native erythropoietin counterpart, wherein the recombinant tissue protective cytokine has cellular protective activity as described herein. Such cellular protective activity includes, but is not limited to, neuroprotective activity. The invention is further directed to a use of any of the aforementioned recombinant tissue protective cytokines in the treatment of a responsive cell, tissue or organ, in particular for treatment of a condition or disease involving such a responsive cell, tissue or organ. In one such embodiment, the recombinant tissue protective cytokines have at least one erythropoietic activity selected from the group consisting of increasing hematocrit, vasoactive action (vasoconstriction/vasodilatation), hyperactivating platelets, pro-coagulant activities and increasing production of thrombocytes. A recombinant tissue protective cytokine of the invention preferably maintains the three-dimensional conformation of native erythropoietin. The recombinant tissue protective cytokine may or may not have erythropoietic activity.

In one embodiment of the invention, the recombinant tissue protective cytokine is created as a recombinant protein with N terminal fusion of HisTag (6×His residues). In certain embodiments, additional amino acid sequences may be added as a spacer. In a specific embodiment, the histidine-tagged recombinant tissue protective cytokine muteins of the invention, include, but are not limited to, K45D-6×His and S100E-6×His.

In another aspect of the invention, any of the foregoing recombinant tissue protective cytokines can be used in the preparation of a pharmaceutical composition for ex vivo treatment of cells, tissues, and organs for the purpose of protecting, maintaining, enhancing, or restoring the function or viability of responsive mammalian cells and their associated cells, tissues, and organs. Such ex vivo treatment is useful, for example, for the preservation of cells, tissues, or organs for transplant, whether autotransplant or xenotransplant. The cells, tissue or organ may be bathed in a solution comprising erythropoietin muteins or recombinant tissue protective cytokines, or the perftisate instilled into the organ through the vasculature or other means, to maintain cellular functioning during the period wherein the cells, tissue or organ is not integrated with the vasculature of the donor or recipient. Administration of the perfusate may be made to a donor prior to organ harvesting, as well as to the harvested organ and to the recipient. Moreover, the aforementioned use of any recombinant tissue protective cytokine is useful whenever a cell, tissue or organ is isolated from the vasculature of the individual and thus essentially existing ex vivo for a period of time, the term isolated referring to restricting or clamping the vasculature of or to the cell, tissue, organ or bodily part, such as may be performed during surgery, including, in particular, cardio pulmonary bypass surgery; bypassing the vasculature of the cell, tissue, organ or bodily part; removing the cell, tissue, organ or bodily part from the mammalian body, such may be done in advance of xenotransplantation or prior to and during autotransplantation; or traumatic amputation of a cell, tissue, organ or bodily part. Thus, this aspect of the invention pertains both to the perfusion with an erythropoietin mutein in situ and ex vivo. Ex vivo, the recombinant tissue protective cytokine may be provided in a cell, tissue or organ preservation solution. For either aspect, the exposing may be by way of continuous perfusion, pulsatile perfusion, infusion, bathing, injection, or catheterization.

In yet a further aspect, the invention is directed to a method for protecting, maintaining, enhancing, or restoring the viability of a mammalian cell, tissue, organ or bodily part which includes a responsive cell or tissue, in which the cell, tissue, organ or bodily part is isolated from the mammalian body. The method includes at least exposing the isolated mammalian cell, tissue, organ or bodily part to an amount of an erythropoietin mutein or recombinant tissue protective cytokine for a duration which is effective to protect, maintain, enhance, or restore the aforesaid viability. In non-limiting examples, isolated refers to restricting or clamping the vasculature of or to the cell, tissue, organ or bodily part, such as may be performed during surgery, in particular, cardio pulmonary bypass surgery; bypassing the vasculature of the cell, tissue, organ or bodily part; removing the cell, tissue, organ or bodily part from the mammalian body, such may be done in advance of xenotransplantation or prior to and during autotransplantation; or traumatic amputation of a cell, tissue, organ or bodily part. Thus, this aspect of the invention pertains both to the perfusion with an erythropoietin mutein or recombinant tissue protective cytokine in situ and ex vivo. Ex vivo, the recombinant tissue protective cytokine may be provided in a cell, tissue or organ preservation solution. For either aspect, the exposing may be by way of continuous perfusion, pulsatile perfusion, infusion, bathing, injection, or catheterization.

By way of non-limiting examples, the aforementioned ex vivo responsive cell or tissue may be or comprise neuronal, retinal, muscle, heart, lung, liver, kidney, small intestine, adrenal cortex, adrenal medulla, capillary endothelial, testis, ovary, pancreas, bone, bone marrow, skin, umbilical chord blood, or endometrial cells or tissue. These examples of responsive cells are merely illustrative.

All of the foregoing methods and uses are preferably applicable to human beings, but are useful as well for any mammal, such as, but not limited to, companion animals, domesticated animals, livestock and zoo animals. Routes of administration of the aforementioned pharmaceutical compositions include oral, intravenous, intranasal, topical, intraluminal, inhalation or parenteral administration, the latter including intravenous, intraarterial, subcutaneous, intramuscular, intraperitoneal, submucosal or intradermal. For ex vivo use, a perfusate or bath solution is preferred. This includes perfusing an isolated portion of the vasculature in situ.

In yet another aspect of the invention, any of the aforementioned recombinant tissue protective cytokines are useful in preparing a pharmaceutical composition for restoring a dysfunctional cell, tissue or organ when administered after the onset of the disease or condition responsible for the dysfunction. By way of non-limiting example, administration of a pharmaceutical composition comprising a recombinant tissue protective cytokine restores cognitive function in animals previously having brain trauma, even when administered long after (e.g., one day, three days, five days, a week, a month, or longer) the initial trauma. The present invention encompasses pharmaceutical compositions for the treatment (i.e. ameliorating or reversing the symptoms or effects of ) and prevention, (i.e. delaying the onset of, inhibiting, or stopping) of subsequent damage to cells and tissues that cascades from initial trauma. Recombinant tissue protective cytokines useful for such applications include any of the particular aforementioned recombinant tissue protective cytokines. Any form of a recombinant tissue protective cytokine capable of benefiting responsive cells is embraced in this aspect of the invention.

In yet another embodiment, the invention provides methods for the use of the aforementioned recombinant tissue protective cytokine for restoring a dysfunctional cell, tissue or organ when administered after the onset of the disease or condition responsible for the dysfunction. By way of non-limiting example, methods for administration of a pharmaceutical composition comprising a recombinant tissue protective cytokine restores cognitive function in animals previously having brain trauma, even when administered long after (e.g., three days, five days, a week, a month, or longer) the trauma has subsided. Recombinant tissue protective cytokines and further modifications thereof are as herein above described. Any form of a recombinant tissue protective cytokine capable of benefiting responsive cells is embraced in this aspect of the invention.

In still yet a further aspect of the present invention, methods are provided for facilitating the transcytosis of a molecule across an endothelial cell barrier in a mammal by administration of a composition of a molecule in association with an erythropoietin mutein or a recombinant tissue protective cytokine as herein before described.

The association between the molecule to be transported and the recombinant tissue protective cytokine may be, for example, a labile covalent bond, a stable covalent bond, or a noncovalent association with a binding site for the molecule. The recombinant tissue protective cytokine and a protein to be transported may be expressed as a fusion polypeptide. Endothelial cell barriers may be the blood-brain barrier, the blood-heart barrier, the blood-kidney barrier, the blood-eye barrier, the blood-testis barrier, the blood-ovary barrier and the blood-placenta barrier. Suitable molecules for transport by the method of the present invention include hormones, such as growth hormone, antibiotics, and anti-cancer agents.

It is a further aspect of the present invention to provide a composition for facilitating the transcytosis of a molecule across an endothelial cell barrier in a mammal, said composition comprising said molecule in association with a recombinant tissue protective cytokine such as is described above.

In a still further aspect of the present invention, any of the aforementioned recombinant tissue protective cytokines are useful in preparing a pharmaceutical composition for facilitating the transcytosis of a molecule across an endothelial cell barrier in a mammal, said composition comprising said molecule in association with a recombinant tissue protective cytokine as described herein above.

The association may be, for example, a labile covalent bond, a fusion polypeptide, a stable covalent bond, or a noncovalent association with a binding site for the molecule. Endothelial cell barriers may be the blood-brain barrier, the blood-eye barrier, the blood-testes barrier, the blood-ovary barrier, and the blood-placenta barrier. Suitable molecules for transport by the method of the present invention include, for example, hormones, such as growth hormone, neurotrophic factors, antibiotics, antivirals, or antifungals such as those normally excluded from the brain and other barriered organs, peptide radiopharmaceuticals, antisense drugs, antibodies against biologically-active agents, pharmaceuticals, dyes, markers, and anti-cancer agents

These and other aspects of the present invention will be better appreciated by reference to the following Figures and Detailed Description.

### 4. BRIEF DESCRIPTION OF THE FIGURES

FIG. 1 shows the distribution of erythropoietin receptor in a normal human brain, in thin sections stained with an anti-erythropoietin antibody.

FIG. 2 is a higher power magnification of the image in FIG. 1.

FIG. 3 shows, using gold-labeled secondary antibodies, the ultramicroscopic distribution of erythropoietin receptors.

FIG. 4, prepared similarly to FIG. 3, shows high density erythropoietin receptors at the luminal and anti-luminal surfaces of human brain capillaries.

FIG. 5 depicts the translocation of parenterally-administered erythropoietin into the cerebrospinal fluid.

FIGS. 6A and 6B indicates the results of the SK-N-SH neuroblastoma cell neuroprotection assay (against rotenone) for erythropoietin as well as the recombinant tissue protective cytokines with the K45D and S100E recombinant tissue protective cytokines. The y-axis on the graph indicates the absorbance readings, and the data are means ± range of duplicate determinations. The graph within FIG. 6A clearly indicates that the viability of the cells within the K45D and S100E samples maintained their viability demonstrating their cellular protective effect. FIG. 6B shows the plasmid map of hEPO-6×HisTag-PCiNeo.

FIG. 7 compares the in vitro efficacy of erythropoietin and asialoerythropoietin on the viability of serum-starved P19 cells.

FIG. 8 is another experiment which compares the in vitro efficacy of erythropoietin and asialoerythropoietin on the viability of serum-starved P19 cells.

FIG. 9 shows protection of erythropoietin and asialoerythropoietin in a rat focal cerebral ischemia model.

FIG. 10 shows a dose response comparing the efficacy of human erythropoietin and human asialoerythropoietin in middle cerebral artery occlusion in a model of ischemic stroke.

FIG. 11 shows the activity of iodinated erythropoietin in the P19 assay.

FIG. 12 shows the effect of biotinylated erythropoietin and asialoerythropoietin in the P19 assay.

FIG. 13 compares the in vitro efficacy of erythropoietin with phenylglyoxal-modified erythropoietin on the viability of serum-starved P19 cells.

FIG. 14 shows the effect of tissue protective cytokines in the water intoxication assay.

FIG. 15 shows the maintenance of the function of a heart prepared for transplantation by an erythropoietin.

FIG. 16 shows the protection of the myocardium from ischemic damage by erythropoietin after temporary vascular occlusion.

FIG. 17 depicts the effects of a erythropoietin treatment in a rat glaucoma model.

FIG. 18 shows the extent of preservation of retinal function by an erythropoietin in the rat glaucoma model.

FIG. 19 depicts the restoration of cognitive function following brain trauma by administration of an erythropoietin starting five days after trauma.

FIG. 20 depicts the restoration of cognitive function following brain trauma by administration of an erythropoietin starting 30 days after trauma.

FIG. 21 depicts the efficacy of human asialoerythropoietin in a kainate model of cerebral toxicity.

FIG. 22 depicts the efficacy of tissue protective cytokines in a rat spinal cord injury model.

FIG. 23 shows the efficacy of tissue protective cytokines within a rabbit spinal cord injury model.

FIG. 24 shows a coronal section of the brain cortical layer stained by hematoxylin and eosin.

FIG. 25 shows coronal sections of frontal cortex adjacent to the region of infarction stained by GFAP antibody.

FIG. 26 shows coronal sections of brain cortical layer stained by OX-42 antibody.

FIG. 27 shows coronal sections of brain cortical layer adjacent to the region of infarction stained by OX-42 antibody.

FIG. 28 shows the efficacy of an erythropoietin against inflammation in an EAE model.

FIG. 29 compares the affects of dexamethasone and an erythropoietin on inflammation in the EAE model.

FIG. 30 shows that erythropoietin suppresses inflammation associated with neuronal death.

FIG. 31 shows that human erythropoietin and recombinant tissue protective cytokines R130E and R150E effectively reduce cell death induced by NMDA when added to the primary hippocampal neuron cell cultures prior to NMDA treatment. Cells treated with R103E (5 nM) exhibited significantly less cell death in comparison to vehicle control cells (p=0.01). Cells treated with R103E (5 nM) exhibited significantly less cell death in comparison to vehicle control cells (p=0.01). Cells treated with R150E (5 nM) exhibited approximately a 20% decrease in cell death in comparison to solvent control cells (p=0.001). Statistics: ANOVA plus Tukey's post-hoc test.

FIG. 32 shows neuronal protection from serum withdrawal in P19 cells. The percent of apaptotic cells decreased for cells pretreated with Epo, EpoWT, and recombinant tissue protective cytokine S100E. Cells treated with Epo exhibited approximately a 20% decrease in apoptotic cell death in comparison to untreated control cells. Cells treated with EpoWT and S100E both exhibited approximately a 10% decrease in apoptotic cell death in comparison to untreated control cells.

FIGS. 33A and 33B Show the effect of pre-incubation with S100E in differentiated PC 12 cells submitted to NGF withdrawal in two independent experiments. Differentiated PC12 cells were pre-treated with S100E at the indicated concentrations for 24 h, FIG. 33A (3 pM) FIG. 33B (0.00003 pM-3 pM). Viability was measured in the MTT assay. NGF (100 ng/ml) was used as a positive control and NGF-free medium (−NGF) as a negative control. Data presented in FIG. 33 are presented as % viability of positive control (+NGF) (n=8 in both experiments). There is a statistically significant increase in viability of S100E treated cells compared to negative control cells (−NGF) by use of one-way ANOVA and Bonferroni post-hoc test. ***p<0.001, *p<0.05. The effects observed with S100E were similar to those of Epo in this test system with respect to potency and efficacy.

FIG. 34 Shows the effect of pre-incubation with Epo in differentiated PC12 cells submitted to NGF withdrawal. Differentiated PC12 cells were pre-treated with Epo, S100E, or carbamylated Epo (30 pM-30 nM) for 24 h. The chemically modified Epo molecule, AA24496, has a 10000 times lower activity than EPO in the UT-7 cell assay. Viability was measured in the MTT assay. NGF (100 ng/ml) was used as a positive control and NGF-free medium (−NGF) as a negative control.

FIG. 35 shows concentration-response curves of Epo, K45D and S100E in UT-7 cells. Different concentrations of Epo, EpoWT, K45D and S100E were added to UT-7 cells. Viability was measured 48 h later in the WST-1 assay. Data are mean ± SD of three different experiments each performed in duplicate. The curve is a non-linear regression curve fit.

FIG. 36 shows dose response curves of Epo, R103E and R150E in UT-7 cells. Different concentrations of Epo, EpoWT, R103E and R150E were added to UT-7 cells. Viability was measured 48 h later in the WST-1 assay. Data are mean ± SD of three different experiments each performed in duplicate. The curve is a non-linear regression curve fit.

FIG. 37 is a graph demonstrating the locomotor ratings of the rats recovering from the spinal cord trauma over a period of forty-two days. As can be seen from the graph, the rats that were given S100E recovered from the injury more readily and demonstrated better overall recovery from the injury than the control rats and rats administered methylprednisolone.

FIG. 38 shows the ratio of the latency of the injured eye over the latency the normal eye for the various treatment regimens. The rat treated with EPO exhibited a latency of 1.2, which is better than the rat treated with saline. Each of the four recombinant tissue protective cytokines resulted in latency results equal to or better than EPO with R103E, R150E, and S100E showing a statistical improvement over EPO.

### 5. DETAILED DESCRIPTION OF THE INVENTION

The present invention relates to mutein recombinant tissue protective cytokines. In particular, the present invention provides compositions comprising isolated nucleic acid molecules encoding recombinant tissue protective cytokine muteins, as well as isolated and/or recombinant cells and vectors comprising the nucleic acid molecules. The invention further encompasses isolated polypeptides of mutein recombinant tissue protective cytokine, lacking at least one erythropoietic activity selected from the group consisting of increasing hematocrit, vasoactive action (vasoconstriction/vasodilatation), hyperactivating platelets, pro-coagulant activities and increasing production of thrombocytes, the cytokine having at least one responsive cellular protective activity selected from the group consisting of protecting, maintaining, enhancing or restoring the function or viability of a responsive mammalian cell, tissue, or organ. The invention also encompasses methods for protecting, maintaining or enhancing the viability of a cell, tissue, or organ isolated from a mammalian body using the recombinant tissue protective cytokine muteins of the invention, and use of such muteins in treatment and prevention of diseases and conditions.

“Responsive cell” refers to a mammalian cell whose function or viability may be maintained, promoted, enhanced, regenerated, or in any other way benefited, by exposure to an erythropoietin. Non-limiting examples of such cells include neuronal, retinal, muscle, heart, lung, liver, kidney, small intestine, adrenal cortex, adrenal medulla, capillary endothelial, testes, ovary, pancreas, bone, skin, and endometrial cells. In particular, responsive cells would include, without limitation, neuronal cells; Purkinje cells; retinal cells: photoreceptor (rods and cones), ganglion, bipolar, horizontal, amacrine, and Müeller cells; muscle cells; heart cells: myocardium, pace maker, sinoatrial node, sinus node, and junction tissue cells (atrioventricular node and bundle of his); lung cells; liver cells: hepatocytes, stellate, and Kupffer cells; kidney cells: mesangial, renal epithelial, and tubular interstitial cells; small intestine cells: goblet, intestinal gland (crypts) and enteral endocrine cells; adrenal cortex cells: glomerulosa, fasciculate, and reticularis cells; adrenal medulla cells: chromaffin cells; capillary cells: pericyte cells; testes cells: Leydig, Sertoli, and sperm cells and their precursors; ovary cells: Graffian follicle and primordial follicle cells; pancreas cells: islets of Langerhans, α-cells, β-cells, γ-cells, and F-cells; bone cells: osteoprogenitors, osteoclasts, and osteoblasts; skin cells; endometrial cells: endometrial stroma and endometrial cells; as well as the stem and endothelial cells present in the above listed organs. Moreover, such responsive cells and the benefits provided thereto by a recombinant tissue protective cytokine may be extended to provide protection or enhancement indirectly to other cells that are not directly responsive, or of tissues or organs which contain such non-responsive cells. These other cells, tissues, or organs which benefit indirectly from the enhancement of responsive cells present as part of the cells, tissue or organ as “associated” cells, tissues, and organs. Thus, benefits of a recombinant tissue protective cytokine as described herein may be provided as a result of the presence of a small number or proportion of responsive cells in a tissue or organ, for example, excitable or neuronal tissue present in such tissue, or the Leydig cells of the testis, which make testosterone. In one aspect, the responsive cell or its associated cells, tissues, or organs are not excitable cells, tissues, or organs, or do not predominantly comprise excitable cells or tissues.

The methods of the invention provide for the local or systemic protection or enhancement of cells, tissues, and organs within a mammalian body, under a wide variety of normal and adverse conditions, or protection of those which are destined for relocation to another mammalian body. In addition, restoration or regeneration of dysfunction is also provided. As mentioned above, the ability of an erythropoietin mutein or a recombinant tissue protective cytokine to cross a tight endothelial cell barrier and exert its positive effects on responsive cells (as well as other types of cells) distal to the vasculature offers the potential to prevent as well as treat a wide variety of conditions and diseases which otherwise cause significant cellular and tissue damage in an animal, including human beings, and moreover, permit success of heretofore untenable surgical procedures for which risk traditionally outweighed the benefits. The duration and degree of purposeful adverse conditions induced for ultimate benefit, such as high-dose chemotherapy, radiation therapy, prolonged ex vivo transplant survival, and prolonged periods of surgically-induced ischemia, may be carried out by taking advantage of the invention herein. However, the invention is not so limited, but includes as one aspect, methods or compositions wherein the target responsive cells are distal to the vasculature by virtue of an endothelial-cell barrier or endothelial tight junctions. In general, the invention is directed to any responsive cells and associated cells, tissues, and organs which may benefit from exposure to a recombinant tissue protective cytokine. Furthermore, cellular, tissue or organ dysfunction may be restored or regenerated after an acute adverse event (such as trauma) by exposure to a recombinant tissue protective cytokine.

The invention is therefore directed generally to the use of recombinant tissue protective cytokines for the preparation of pharmaceutical compositions for the aforementioned purposes in which cellular function is maintained, promoted, enhanced, regenerated, or in any other way benefited. The invention is also directed to methods for maintaining, enhancing, promoting, or regenerating cellular function by administering to a mammal an effective amount of a recombinant tissue protective cytokine as described herein. The invention is further directed to methods for maintaining, promoting, enhancing, or regenerating cellular function ex vivo by exposing a cell, a tissue or an organ to a recombinant tissue protective cytokine. The invention is also directed to a perfusate composition comprising a recombinant tissue protective cytokine for use in organ or tissue preservation.

The various methods of the invention utilize a pharmaceutical composition which at least includes a recombinant tissue protective cytokine at an effective amount for the particular route and duration of exposure to exert positive effects or benefits on responsive cells within or removed from a mammalian body. Where the target cell, tissues, or organs of the intended therapy require the recombinant tissue protective cytokine to cross an endothelial cell barrier, the pharmaceutical composition includes the recombinant tissue protective cytokine at a concentration which is capable, after crossing the endothelial cell barrier, of exerting its desirable effects upon the responsive cells. Molecules capable of interacting with an erythropoietin receptor, and modulating cellular protective activity within the cell are useful in the context of the present invention.

### 5.1. NUCLEIC ACIDS OF THE INVENTION

A recombinant tissue protective cytokine comprising a nucleic acid molecule of the invention includes nucleic acids encoding tissue protective cytokines comprising an erythropoietin mutein lacking or exhibiting a decrease in at least one erythropoietic activity selected from the group consisting of increasing hematocrit, vasoactive action (vasoconstriction/vasodilatation), hyperactivating platelets, pro-coagulant activities and increasing production of thrombocytes, the cytokine having at least one responsive cellular protective activity selected from the group consisting of protecting, maintaining, enhancing or restoring the function or viability of a responsive mammalian cell, tissue or organ. A tissue protective cytokine comprising a nucleic acid molecule of the invention includes nucleic acids encoding the erythropoietin mutein, with the activity described above, comprising one or more altered amino acid residue between position 11-15 of SEQ ID NO:10 [SEQ ID NO:1], position 44-51 of SEQ ID NO 10 [SEQ ID NO:2], position 100-108 of SEQ ID NO [SEQ ID NO:3], or position 146-151 of SEQ ID NO 10 [SEQ ID NO:4]. A tissue protective cytokine comprising a nucleic acid molecule of the invention includes nucleic acids encoding the erythropoietin mutein, with the activity described above, comprising an altered amino acid residue at one or more of the following positions of SEQ ID NO:10: 7, 20, 21, 29, 33, 38, 42, 59, 63, 67, 70, 83, 96, 126, 142, 143, 152, 153, 155, 156, or 161. A tissue protective cytokine comprising a nucleic acid molecule of the invention includes nucleic acids encoding the erythropoietin mutein, with the activity described above, comprising the amino acid sequence of SEQ ID NO:10 with one or more of the following changes: an alanine at residue 6 of SEQ ID NO:10, an alanine at residue 7 of SEQ ID NO:10, a serine at residue 7 of SEQ ID NO:10, an isoleucine at residue 10 of SEQ ID NO:10, a serine at residue 11 of SEQ ID NO:10, an alanine at residue 12 of SEQ ID NO:10, an alanine at residue 13 of SEQ ID NO:10, an alanine residue 14 of SEQ ID NO:10, a glutamic acid at residue 14 of SEQ ID NO:10, a glutamine at residue 14 of SEQ ID NO:10, an alanine at residue 15 of SEQ ID NO:10, a phenylalanine at residue 15 of SEQ ID NO:10, an isoleucine at residue 15 of SEQ ID NO:10, a glutamic acid at residue 20 of SEQ ID NO:10, an alanine at residue 20 of SEQ ID NO:10, an alanine at residue 21 of SEQ ID NO:10, a lysine at residue 24 of SEQ ID NO:10, a serine at residue 29 of SEQ ID NO:10; a tyrosine at residue 29 of SEQ ID NO:10, an asparagine at residue 30 of SEQ ID NO:10, a threonine at residue 32 of SEQ ID NO:10, a serine at residue 33 of SEQ ID NO:10, a tyrosine at residue 33 of SEQ ID NO:10, a lysine at residue 38 of SEQ ID NO: 10, a lysine at residue 83 of SEQ ID NO:10, an asparagine at residue 42 of SEQ ID NO:10, an alanine at residue 42 of SEQ ID NO:10, an alanine at residue 43, an isoleucine at residue 44 of SEQ ID NO:10, an aspartic acid at residue 45 of SEQ ID NO:10, an alanine at residue 45 of SEQ ID NO:10, an alanine at residue 46 of SEQ ID NO:10, an alanine at residue 47 of SEQ ID NO:10, an isoleucine at residue 48 of SEQ ID NO:10, an alanine at residue 48 of SEQ ID NO:10, an alanine at residue 49 of SEQ ID NO:10, a serine at residue 49 of SEQ ID NO:10, a phenylalanine at residue 51 of SEQ ID NO:10, an asparagine at residue 51 of SEQ ID NO:10, an alanine at residue 52 of SEQ ID NO:10, an asparagine at residue 59 of SEQ ID NO:10, a threonine at residue 62 of SEQ ID NO:10, a serine at residue 67 of SEQ ID NO:10, an alanine at residue 70 of SEQ ID NO:10, an arginine at residue 96 of SEQ ID NO:10, an alanine at residue 97 of SEQ ID NO:10, an arginine at residue 100 of SEQ ID NO:10, a glutamic acid at residue 100 of SEQ ID NO: 10 of SEQ ID NO:10, an alanine at residue 100, a threonine at residue 100 of SEQ ID NO: 10, an alanine at residue 101 of SEQ ID NO:10, an isoleucine at residue 101 of SEQ ID NO:10, an alanine at residue 102 of SEQ ID NO:10, an alanine at residue 103 of SEQ ID NO:10, a glutamic acid at residue 103 of SEQ ID NO:10, an alanine at residue 104 of SEQ ID NO:10, an isoleucine at residue 104 of SEQ ID NO:10, an alanine at residue 105 of SEQ ID NO:10, an alanine at residue 106 of SEQ ID NO:10, an isoleucine at residue 106 of SEQ ID NO:10, an alanine at residue 107 of SEQ ID NO:10, a leucine at residue 107 of SEQ ID NO:10, a lysine at residue 108 of SEQ ID NO:10, an alanine at residue 108 of SEQ ID NO:10, a serine at residue 108 of SEQ ID NO:10, an alanine at residue 116 of SEQ ID NO:10, an alanine at residue 126 of SEQ ID NO:10, an alanine at residue 132 of SEQ ID NO:10, an alanine at residue 133 of SEQ ID NO:10, an alanine at residue 134 of SEQ ID NO:10, an alanine at residue 140 of SEQ ID NO:10, an isoleucine at residue 142 of SEQ ID NO:10, an alanine at residue 143 of SEQ ID NO:10, an alanine at residue 146 of SEQ ID NO:10, a lysine at residue 147 of SEQ ID NO:10, an alanine at residue 147 of SEQ ID NO:10, a tyrosine at residue 148 of SEQ ID NO:10, an alanine at residue 148 of SEQ ID NO:10, an alanine at residue 149 of SEQ ID NO:10, an alanine at residue 150 of SEQ ID NO:10, a glutamic acid at residue 150 of SEQ ID NO:10, an alanine at residue 151 of SEQ ID NO:10, an alanine at residue 152 of SEQ ID NO:10, a tryptophan at residue 152 of SEQ ID NO:10, an alanine at residue 153 of SEQ ID NO:10, an alanine at residue 154 of SEQ ID NO:10, an alanine at residue 155 of SEQ ID NO:10, an alanine at residue 158 of SEQ ID NO:10, a serine at residue 160 of SEQ ID NO:10, an alanine at residue 161 of SEQ ID NO:10, or an alanine at residue 162 of SEQ ID NO:10.

The nucleic acid molecules of the invention further include nucleotide sequences that encode recombinant erythropoietin muteins having at least 30%, 35%, 40%, 45%, 50%, 55%, 60%, 65%, 70%, 75%, 80%, 85%, 90%, 95%, 98%, or higher amino acid sequence identity to one of the erythropoietin muteins described above. To determine the percent identity of two amino acid sequences or of two nucleic acids encoding erythropoietin muteins, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of overlapping positions=100%). In one embodiment, the two sequences are the same length.

The nucleic acid molecules of the invention further include nucleotide sequences that encode recombinant erythropoietin muteins wherein the erythropoietin encoding nucleic acid sequence that is altered by one or more of the substitutions, deletions, or modifications described above comprises at least 30%, 35%, 40%, 45%, 50%, 55%, 60%, 65%, 70%, 75%, 80%, 85%, 90%, 95%, or 98% sequence identity to SEQ ID NO:7. The nucleic acid molecules of the invention also include nucleotide sequences that encode recombinant erythropoietin muteins wherein the erythropoietin encoding nucleic acid sequence that is altered by one or more of the substitutions, deletions, or modifications described above is a non-human erythropoietin encoding nucleic acid.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The nucleic acid molecules of the invention further include: (a) any nucleotide sequence that hybridizes to an erythropoietin mutein or a recombinant tissue protective cytokine encoding nucleic acid molecule of the invention described above, under stringent conditions, e.g., hybridization to filter-bound DNA in 6× sodium chloride/sodium citrate (SSC) at about 45° C. followed by one or more washes in 0.2×SC/0.1% SDS at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the encoding erythropoietin mutein nucleic acid molecule that hybridizes under conditions described under (a) and (b), above, is one that comprises the complement of a nucleic acid molecule that encodes a erythropoietin mutein. In a preferred embodiment, nucleic acid molecules that hybridize under conditions (a) and (b), above, encode protein products, e.g., protein products functionally equivalent, i.e. having one or more of the activities of erythropoietin described above, to an erythropoietin mutein. Preferably, the nucleic acids of the invention are human.

The nucleic acid molecules of the invention further include the above nucleotide sequences that hybridize to a erythropoietin mutein or a recombinant tissue protective cytokine as described above and farther lack or exhibit a decrease in at least one erythropoietic activity selected from the group consisting of increasing hematocrit, vasoactive action (vasoconstriction/vasodilatation), hyperactivating platelets, pro-coagulant activities and increasing production of thrombocytes, the cytokine or mutein comprising at least one responsive cellular protective activity selected from the group consisting of protecting, maintaining, enhancing or restoring the function or viability of a responsive mammalian cell, tissue or organ. The decrease may be a slight diminishment or near lack of one of the erythropoietic activities. Such decreases can be measured by standard techniques known in the art (Gruber et al., 2002, J. Biol Chem. 277(81):27581-27584; Page et al., 1996, Cytokine 8(1):66-69; Park et al., 1997, Mol. Cells 7(6):699-704; Wolf et al., 1997, Thromb Haemost 78:1505-1509; and Dale et al., 2002, Nature 415:175-179. The UT-7 cell assays described in Section 6.17 are one, non-limiting, example of a technique to measure decreased or diminished erythropoietic activity.

The nucleic acid molecules of the invention further comprise the complements of the nucleic acids described above.

Fragments of the erythropoietin mutein nucleic acid molecules refer to erythropoietin mutein nucleic acid sequences described above that can be at least 10, 12, 15, 20, 30, 40, 50, 60, 70, 80, 90, 100, 200, 300, 400, 500, 600, 700, 800, 900, 1000, 1050, or more contiguous nucleotides in length. Alternatively, the fragments can comprise sequences that encode at least 10, 20, 30, 40, 50, 60, 70, 80 or more contiguous amino acid residues of the erythropoietin mutein. In one embodiment, the erythropoietin mutein nucleic acid molecule encodes a gene product exhibiting at least one biological activity of a corresponding erythropoietin mutein. Fragments of the erythropoietin mutein nucleic acid molecules can also refer to portions of erythropoietin mutein coding regions that encode domains of, or mature erythropoietin mutein.

Erythropoietin derived from other organisms may be used to create the erythropoietin muteins of the invention. With respect to the cloning of variants of the erythropoietin mutein or recombinant tissue protective cytokine nucleic acids and homologous and orthologs from other species, the isolated erythropoietin nucleic acid sequences disclosed herein may be labeled and used to screen a cDNA library constructed from mRNA obtained from appropriate cells or tissues derived from the organism of interest. The hybridization conditions used should generally be of a lower stringency when the cDNA library is derived from an organism different from the type of organism from which the labeled sequence was derived, and can routinely be determined based on, e.g., relative relatedness of the target and reference organisms.

Alternatively, the labeled fragment may be used to screen a genomic library derived from the organism of interest, again, using appropriately stringent conditions. Appropriate stringency conditions are well known to those of skill in the art as discussed above, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Press, N.Y.; and Ausubel, et al., 1989-1999, Current Protocols in Molecular Biology, Green Publishing Associates and Wiley Interscience, N.Y., both of which are incorporated herein by reference in their entirety.

In a preferred embodiment, to make a recombinant tissue protective cytokine DNA can be amplified from genomic or cDNA (i.e. SEQ ID NO:7) by polymerase chain reaction (PCR) amplification using primers designed from the known sequence of a related or homologous recombinant tissue protective cytokine. PCR is used to amplify the desired sequence in DNA clone or a genomic or cDNA library, prior to selection. PCR can be carried out, e.g., by use of a thermal cycler and Taq polymerase (Gene Amp®). The polymerase chain reaction (PCR) is commonly used for obtaining genes or gene fragments of interest. For example, a nucleotide sequence encoding a recombinant tissue protective cytokine of any desired length can be generated using PCR primers that flank the nucleotide sequence encoding open reading frame. Alternatively, a recombinant tissue protective cytokine gene sequence can be cleaved at appropriate sites with restriction endonuclease(s) if such sites are available, releasing a fragment of DNA encoding the recombinant tissue protective cytokine gene. If convenient restriction sites are not available, they may be created in the appropriate positions by site-directed mutagenesis and/or DNA amplification methods known in the art (see, for example, Shankarappa et al., 1992, PCR Method Appl. 1: 277-278). The DNA fragment that encodes the recombinant tissue protective cytokine is then isolated, and ligated into an appropriate expression vector, care being taken to ensure that the proper translation reading frame is maintained.

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem. 253: 6551), oligonucleotide-directed mutagenesis (Smith, 1985, Ann. Rev. Genet. 19: 423-463; Hill et al., 1987, Methods Enzymol. 155: 558-568) and as described in section 6.3, PCR-based overlap extension (Ho et al., 1989, Gene 77: 51-59), PCR-based megaprimer mutagenesis (Sarkar et al., 1990, Biotechniques 8: 404-407), etc. Modifications can be confirmed, e.g., by double-stranded dideoxynucleotide DNA sequencing.

The invention also includes nucleic acid molecules, preferably DNA molecules, that are the complements of the nucleotide sequences of the preceding paragraphs.

In certain embodiments, the nucleic acid molecules of the invention are present as part of nucleic acid molecules comprising nucleic acid sequences that contain or encode heterologous (e.g., vector, expression vector, or fusion protein) sequences.

### 5.2. RECOMBINANT TISSUE PROTECTIVE CYTOKINES OF THE INVENTION

Recombinant tissue protective cytokines of the invention include erythropoietin muteins, that maintain partial or full erythropoietic activity. Erythropoietin is a glycoprotein hormone which in humans has a molecular weight of about 34 kDa. The mature protein comprises 165 amino acids, and the glycosyl residues comprise about 40% of the weight of the molecule. The forms of recombinant tissue protective cytokine useful in the practice of the present invention encompass at least a single amino acid change in naturally-occurring, synthetic and recombinant forms of the following human and other mammalian erythropoietin-related molecules: erythropoietin, asialoerythropoietin, deglycosylated erythropoietin, erythropoietin analogs, erythropoietin mimetics, erythropoietin fragments, hybrid erythropoietin molecules, erythropoietin receptor-binding molecules, erythropoietin agonists, renal erythropoietin, brain erythropoietin, oligomers and multimers thereof, and congeners thereof. Such equivalent recombinant tissue protective cytokines include mutant erythropoietins, which may contain substitutions, deletions, including internal deletions, additions, including additions yielding fusion proteins, or conservative substitutions of amino acid residues within and/or adjacent to the amino acid sequence, but that result in a “silent” change, in that the change produces a functionally equivalent erythropoietin mutein or recombinant tissue protective cytokine. In a preferred embodiment, the recombinant tissue protective cytokine is nonerythropoietic, i.e. lacking or exhibiting diminished erythropoietic activity. Conservative amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, non-conservative amino acid changes, and larger insertions and deletions may be used to create functionally altered recombinant tissue protective cytokines. Such mutants can be used to alter erythropoietin properties in desirable ways. For example, in one embodiment, an erythropoietin useful for the practice of the invention can be a recombinant tissue protective cytokine altered in one or more amino acids within the four functional domains of erythropoietin which affect receptor binding: VLQRY (SEQ ID NO:1) and/or TKVNFYAW (SEQ ID NO:2) and/or SGLRSLTTL (SEQ ID NO:3) and/or SNFLRG (SEQ ID NO:4). In another embodiment, erythropoietins containing mutations in the surrounding areas of the molecule which affect the kinetics or receptor-binding properties of the molecule can be used. Determining which alterations, or which positions in the domains will effect binding can be accomplished using standard methods. For example, the domains may be altered by pair-wise alanine mutations (ala-scanning mutagenesis) followed by measurement of binding kinetics of mutants to examine the effect on binding to a receptor (Bernat et al., 2003, PNAS 100:952-957; Wells et al., 1989, Science 244:1081-1085).

The term “recombinant tissue protective cytokine” as well as “a recombinant tissue protective cytokine” may be used interchangeably or conjunctively, to encompass the recombinant tissue protective cytokines of the invention and further modifications thereof, such as deglycosylated, asialylated, and other partially glycosylated forms of the recombinant tissue protective cytokine, or chemical modifications of the amino acids. Non-limiting examples of such variants are described in Tsuda et al., 1990, Eur. J. Biochem. 188:405-411, incorporated herein by reference. Cytokines are highly flexible, and, in the case of human growth hormone it is known that flexibility is required for activation (Wells et al., 1989, Science 244:1081-1085). Thus, mutations that stabilize the three dimensional structure of a cytokine, preventing normal activation of the erythropoietin receptor are encompassed by the instant invention. In addition, a variety of host systems may be used for expression and production of recombinant tissue protective cytokines, including, but not limited to, bacteria, yeast, insect, plant, and mammalian, including human, cell systems. For example, recombinant erythropoietin produced in bacteria, which do not glycosylate, asialylate, or partially glycosylate the product, could be used to produce non-glycosylated forms of the recombinant tissue protective cytokine or may be further glycosylated using known methods in the art, such as, but not limited to, those techniques disclosed in U.S. patent application Ser. Nos: U.S. 2003/0040037 A1 and U.S. 2003/0003529 for use of fucosylation to adjust glycosylation of proteins. Alternatively, recombinant tissue protective cytokine can be produced in other systems capable of glycosylating expressed proteins, e.g., plants, and including human cells.

As noted above, the invention herein embraces any and all erythropoietin receptor activity modulator molecules capable of exerting positive activity on responsive cells, regardless of any structural relationship of the molecule with erythropoietin.

In addition, the recombinant tissue protective cytokine may be modified to tailor its activities for a specific tissue or tissues. Several non-limiting strategies which may be carried out to achieve this desired tissue specificity include modifications that shorten circulating half-life and thus reduce the time the recombinant tissue protective cytokine can interact with erythroid precursors, or modification of the primary structure of the erythropoietin mutein or recombinant tissue protective cytokine molecule. One approach to reducing circulating half life is to remove or modify the glycosylation moieties, of which erythropoietin has three N-linked and one O-linked. Such variants of a glycosylated recombinant tissue protective cytokine can be produced in a number of ways. For example, techniques to modify the primary structure of erythropoietin to generate the tissue protective cytokines of the present invention are myriad and include substitution of one or more specific amino acids, i.e., by mutating the amino acids at the N-linked or O-linked glycosylation sites and/or, chemical modification of one or more amino acids, or addition of other structures which interfere with the interaction of erythropoietin with any of its receptors. Use of such forms of recombinant tissue protective cytokines is fully embraced herein. The sialic acids which terminate the end of the sugar chains can be removed by specific sialidases depending on the chemical linkage connecting the sialic acid to the sugar chain. Alternatively, the glycosylated structure can be dismantled in different ways by using other enzymes that cleave at specific linkages. In a preferred embodiment, the half-life of the non-erythropoietic recombinant tissue protective cytokine of the invention is reduced by about 90% from that of native erythropoietin.

Some of these recombinant tissue protective cytokine molecules will nevertheless mimic the actions of erythropoietin itself in other tissues or organs. For example, a 17-mer containing the amino-acid sequence of 31-47 of native erythropoietin is inactive for erythropoiesis but fully active for neural cells in vitro (Campana & O'Brien, 1998: Int. J. Mol. Med. 1:235-41).

Furthermore, derivative recombinant tissue protective cytokine molecules desirable for the uses described herein may be generated by guanidination, amidination, carbamylation (carbamoylation), trinitrophenylation, acylation such as acetylation or succinylation, nitration, or modification of arginine, aspartic acid, glutamic acid, lysine, tyrosine, tryptophan, or cysteine residues or carboxyl groups, among other procedures, such as limited proteolysis, removal of amino groups, and/or mutational substitution of arginine, lysine, tyrosine, tryptophan, or cysteine residues by molecular biological techniques to produce erythropoietin muteins or recombinant tissue protective cytokines which maintain an adequate level of activities for specific organs and tissues but not for others, such as erythrocytes (e.g., Satake et al; 1990, Biochim. Biophys. Acta 1038:125-9; incorporated herein by reference in its entirety). One non-limiting example as described hereinbelow is the modification of erythropoietin arginine residues by reaction with a glyoxal such as phenylglyoxal (according to the protocol of Takahashi, 1977, J Biochem. 81:395-402). As will be seen below, such a recombinant tissue protective cytokine molecule fully retains the neurotrophic effect of erythropoietin. Such recombinant tissue protective cytokine molecules are fully embraced for the various uses and compositions described herein. In addition, these chemical modifications may be further used to enhance the protective effects of the recombinant tissue protective cytokines or neutralize any changes in the charge of the molecule resulting from the amino acid mutation of the native erythropoietin. Such modifications are described in co-pending applications:, Ser. No. PCT/US01/49479, filed Dec. 28, 2001; Ser. No. 09/753,132, filed Dec. 29, 2000 and Attorney's Docket No. KW00-009C02-US, filed Jul. 3, 2002, all of which are incorporated herein in their entireties.

Synthetic and recombinant molecules, such as brain erythropoietin and renal erythropoietin, recombinant mammalian forms of erythropoietin, as well as its naturally-occurring, tumor-derived, and recombinant isoforms, such as recombinantly-expressed molecules and those prepared by homologous recombination are provided herein. Furthermore, the present invention includes molecules including peptides which bind the erythropoietin receptor, as well as recombinant constructs or other molecules which possess part or all of the structural and/or biological properties of erythropoietin, including fragments and multimers of erythropoietin or its fragments. Erythropoietin muteins or other recombinant tissue protective cytokines which have additional or reduced numbers of glycosylation sites are included herein. As noted above, the terms “erythropoietin” and “mimetics” as well as the other terms are used interchangeably herein to refer to the responsive cell protective and enhancing molecules related to erythropoietin as well as the molecules which are capable of crossing endothelial cell barriers. Furthermore, molecules produced by transgenic animals are also encompassed here. It should be noted that erythropoietin molecules as embraced herein do not necessarily resemble erythropoietin structurally or in any other manner, except for ability to interact with the erythropoietin receptor or modulate erythropoietin receptor activity or activate erythropoietin-activated signaling cascades, as described herein.

By way of non-limiting examples, forms of recombinant tissue protective cytokines useful for the practice of the present invention include recombinant tissue protective cytokines, such as those with altered amino acids at the carboxy terminus described in U.S. Pat. No. 5,457,089 and in U.S. Pat. No. 4,835,260; asialoerythropoietin and erythropoietin isoforms with various numbers of sialic acid residues per molecule, such as described in U.S. Pat. No.t 5,856,298; polypeptides described in U.S. Pat. No. 4,703,008; agonists described in U.S. Pat. No. 5,767,078; peptides which bind to the erythropoietin receptor as described in U.S. Pat. Nos. 5,773,569 and 5,830,851; small-molecule mimetics which activate the erythropoietin receptor, as described in U.S. Pat. No. 5,835,382; and erythropoietin analogs described in WO 9505465, WO 9718318, and WO 9818926. All of the aforementioned citations are incorporated herein to the extent that such disclosures refer to the various alternate forms or processes for preparing such forms of the recombinant tissue protective cytokines of the present invention.

Erythropoietin can be obtained commercially, for example, under the trademarks of PROCRIT, available from Ortho Biotech Inc., Raritan, N.J., and EPOGEN, available from Amgen, Inc., Thousand Oaks, Calif.

The activity (in units) of erythropoietin (EPO) and erythropoietin-like molecules is traditionally defined based on its effectiveness in stimulating red cell production in rodent models (and as derived by international standards of erythropoietin). One unit (U) of regular erythropoietin (MW of ˜30,000 to ˜34,000) is - 8 ng of protein (1 mg protein is approximately 125,000 U). However, as the effect on erythropoiesis is incidental to the desired activities herein and may not necessarily be a detectable property of certain of the recombinant tissue protective cytokines of the invention, the definition of activity based on erythropoiesis is inappropriate. Thus, as used herein, the activity unit of erythropoietin or erythropoietin-related molecules is defined as the amount of protein required to elicit the same activity in neural or other responsive cellular systems as is elicited by WHO international standard erythropoietin in the same system. The skilled artisan will readily determine the units of a non-erythropoietic recombinant tissue protective cytokine or related molecule following the guidance herein.

The recombinant tissue protective cytokine muteins include, but are not limited to, those proteins and polypeptides encoded by the erythropoietin nucleic acid sequences described in Section 6.3. The invention encompasses muteins that are functionally equivalent to the erythropoietin gene product described in Section 6.3. Such erythropoietin gene products may contain one or more deletions, additions or substitutions of erythropoietin amino acid residues within the amino acid sequence encoded by an erythropoietin nucleic acid sequence, but which result in a silent change, thus producing a functionally equivalent erythropoietin gene product. Amino acid substitutions may be made on the basis of similarity in polarity, charge, solubility, hydrophobicity, hydrophilicity, and/or the amphipathic nature of the residues involved.

The recombinant tissue protective cytokine muteins of the invention can be generated by mutagenesis, e.g., discrete point mutation or truncation. A recombinant tissue protective cytokine mutein of the invention retains the cellular protective biological activities of the naturally occurring form, but may lack one or more of the erythropoietic activities of the naturally occurring form of the protein. Thus, specific biological effects can be elicited by addition of a mutein of limited function.

Modification of the structure of the recombinant tissue protective cytokine muteins can be for such purposes as enhancing efficacy, stability, or post-translational modifications (e.g., to alter the phosphorylation pattern of the muteins). Such modified recombinant tissue protective cytokine muteins, when designed to retain at least one cellular protective activity of the naturally-occurring form of the protein, or to produce specific antagonists thereof, are considered functional equivalents of the recombinant tissue protective cytokine muteins. Such modified recombinant tissue protective cytokine muteins can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule.

Whether a change in the amino acid sequence of a recombinant tissue protective cytokine mutein results in a functional homolog, or non-functional homolog (i.e. lacking one or more of the activities of the non-mutated cytokine), can be readily determined by assessing the ability of the variant mutein to produce a response in cells in a fashion similar to the wild-type cytokine, or competitively inhibit such a response. Recombinant tissue protective cytokine muteins in which more than one replacement has taken place can readily be tested in the same manner.

Muteins of the invention exhibiting altered function can be identified by screening combinatorial libraries of mutants, e.g., truncation mutants, of the recombinant tissue protective cytokine of the invention for desired activity or lack thereof. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into nucleic acid sequences such that a degenerate set of potential protein sequences is expressible as individual polypeptides, or alternatively, as a set of larger fusion proteins (e.g., for phage display). There are a variety of methods which can be used to produce libraries of potential variants of the recombinant tissue protective cytokines of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983, Nucleic Acid Res.11:477).

In addition, libraries of fragments of the coding sequence of a recombinant tissue protective cytokines of the invention can be used to generate a variegated population of recombinant tissue protective cytokines for screening and subsequent selection of muteins. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the recombinant tissue protective cytokine muteins of interest.

Several techniques are known in the art for screening gene products of combinatorial libraries made by point mutations or truncation, and for screening cDNA libraries for gene products having a selected property. The most widely used techniques, which are amenable to high through put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify muteins of a recombinant tissue protective cytokine of the invention (Arkin and Yourvan, 1992, Proc. Natl. Acad. Sci. USA 89:7811-7815; Delgrave et al., 1993, Protein Engineering 6(3):327-331).

An isolated nucleic acid molecule encoding a mutein can be created by introducing one or more nucleotide substitutions, additions or deletions into the erythropoietin nucleotide sequence, such that one or more amino acid substitutions, additions or deletions are introduced into the encoded recombinant tissue protective cytokine. Mutations can be introduced by standard techniques, such as site-directed mutagenesis and PCR-mediated mutagenesis. Briefly, PCR primers are designed that delete the trinucleotide codon of the amino acid to be changed and replace it with the trinucleotide codon of the amino acid to be included. This primer is used in the PCR amplification of DNA encoding the recombinant tissue protective cytokine of interest. This fragment is then isolated and inserted into the full length cDNA encoding the tissue protective cytokine of interest and expressed recombinantly. The resulting recombinant tissue protective cytokine now includes the amino acid replacement.

Either conservative or non-conservative amino acid substitutions can be made at one or more amino acid residues. Both conservative and non-conservative substitutions can be made. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (See, for example, Biochemistry, 4th ed., Ed. by L. Stryer, WH Freeman and Co.: 1995).

Alternatively, mutations can be introduced randomly along all or part of the coding sequence of a recombinant tissue protective cytokine, such as by saturation mutagenesis, and the resultant mutants can be screened for biological activity to identify mutants that retain activity. Following mutagenesis, the encoded protein can be expressed recombinantly and the activity of the recombinant tissue protective cytokine can be determined.

Further to the above-mentioned erythropoietin modifications useful herein, the following discussion expands on the various recombinant tissue protective cytokines of the invention. As described in Elliott et al., Boissel et al., and Wen et al., mentioned above, the following erythropoietin muteins are useful for the purposes described herein, and may be provided in a pharmaceutical composition for the methods herein. In the mutein nomenclature used throughout herein, the changed amino acid is depicted with the native amino acid's one-letter code first, followed by its position in the erythropoietin molecule, followed by the replacement amino acid one-letter code. For example, “human erythropoietin S100E” or “recombinant tisue protectiv cytokine S100E” refers to a human erythropoietin molecule in which amino acid 100, a serine has been changed to glutamic acid. Such muteins useful for the practice of the present invention include but are not limited to human erythropoietin with at least one of the following amino acid changes:

I6A, C7A, C7S,

R10I, V11S, L12A, E13A, R14A, R14E, R14Q, Y15A, Y15F, Y15I,

K20E,K20A,

E21A,

N24K, C29S, C29Y, A30N, H32T,

C33S, C33Y, N38K, N83K,

P42N,

P42A, D43A, T441, K45D, K45A, V46A, N47A, F481, F48A, Y49A, Y49S, 44-49 deletion,

W51F, W51N, K52A,

Q59N,

E62T,

L67S,

L70A,

D96R, K97A

S100R, S100E, S100A, S100T, G101A, G101I, L102A, R103A, R103E, S104A, S104I,

L105A, T106A, T106I, T107A, T107L, L108K, L108A, L108S,

K116A,

S126A,

T132A,

I133A, T134A,

K140A,

F142I,

R143A,

S146A, N147K, N147A, F148Y, P148A, L149A, R150A, R150E, G151A,

K152A, K152W,

L153A,

K154A,

L155A, G158A,

C160S, C161A, or R162A.

In preferred embodiments, an erythropoietin mutein or a recombinant tissue protective cytokine of the invention comprises one or more of the above substitutions. In other embodiments, erythropoietin mutein or another recombinant tissue protective cytokine of the invention comprises one of the above substitutions or a combination thereof.

In an alternative embodiment, the recombinant tissue protective cytokines, pharmaceutical compositions, use, and treatment methods of the invention comprise one or more of the above substitutions with the proviso that they do not comprise one or more of the following substitutions: I6A, C7A, K20A, P42A, D43A, K45D, K45A, F48A, Y49A, K52A, K49A, S100E, R103A, K116A, T132A, I133A, K140A, N147K, N147A, R150A, R150E, G151A, K152A, K154A, G158A, C161A, or R162A. In a related embodiment of the invention, the recombinant tissue protective cytokines, pharmaceutical compositions, use, and treatment methods of the invention comprise one or more of the above substitutions with the proviso that they do not comprise any of the following combinations of substitutions: N24K/N38K/N83K or A30N/H32T.

In certain embodiments, more than one of the amino acid changes above can be combined to make a mutein. Examples of such combinations include, but are not limited to: K45D/S100A30N/H32T, K45D/R150E, R103E/L108S, K140A/K52A, K140A/K52A/K45A, K97A/K152A, K97A/K152A/K45A, K97A/K152A/K45A/K52A, K97A/K152A/K45A/K52A/K140A, K97A/K152A/K45A/K52A/K140A/K154A, N24K/N38K/N83K, and N24K/Y15A. In certain embodiments, the recombinant tissue protective cytokine mutein of the invention does not comprise one or more of the above multiple substitutions. In certain embodiments the pharmaceutical compositions of the invention comprising the recombinant tissue protective cytokine mutein of the invention do not comprise one or more of the above multiple substitutions. In certain embodiments the use and treatment methods of the invention which utilize the recombinant tissue protective cytokine mutein of the invention do not comprise one or more of the above multiple substitutions.

Certain modifications or combinations of modifications can effect the flexibility of a erythropoietin muteins effecting binding to a receptor, such as the erythropoietin receptor or a secondary receptor to which erythropoietin or an erythropoietin mutein binds. Examples of such modifications or combinations thereof useful in the compositions and methods of the invention, include, but are not limited to, K152W, R14A/Y15A, I6A, C7A, D43A, P42A, F48A, Y49A, T132A, I133A, T134A, N147A, P148A, R150A, G151A, G158A, C161A, and R162A. Corresponding mutations are known to be detrimental in human growth hormone (Wells et al.). In certain embodiments, the recombinant tissue protective cytokine mutein of the invention does not comprise one or more of the above substitutions. In certain embodiments the pharmaceutical compositions of the invention comprising the recombinant tissue protective cytokine mutein of the invention do not comprise one or more of the above substitutions. In certain embodiments the use and treatment methods of the invention which utilize the recombinant tissue protective cytokine mutein of the invention do not comprise one or more of the above substitutions.

In addition to one of the foregoing amino acid modifications, a recombinant tissue protective cytokine of the invention may also have at least no sialic acid moieties, referred to as an asialoerythropoietin mutein. Preferably, an asialoerythropoietin mutein of the invention is human asialoerythropoietin. In alternative embodiments, the recombinant tissue protective cytokine of the invention may have at least 1, 2, 3, 4, 5, 6, 7, 8, 9, 10, 11, 12, or 13 sialic acid residues. It may be prepared by desialylating a recombinant tissue protective cytokine using a sialidase, such as is described in the manufacturer's packaging for Sialydase A from ProZyme Inc., San Leandro, Calif. Typically, PROZYME® GLYCOPRO® sequencing-grade SIALYDASE A™ (N-acetylneuraminate glycohydrolase, EC 3.2.1.18) is used to cleave all non-reducing terminal sialic acid residues from complex carbohydrates and glycoproteins such as erythropoietin. It will also cleave branched sialic acids (linked to an internal residue). Sialydase A is isolated from a clone of Arthrobacter ureafaciens.

A non-limiting example of sialylation of a glycopeptide is found in U.S. patent application Ser. No. U.S. 2003/0040037, which discloses methods of sialylation using mammalian or bacterial sialytransferases. Another non-limiting example of methods for sialylation and alteration of sialylation patterns on glycoproteins is found in U.S. patent application Ser. No. U.S. 2002/0160460 A1 and in U.S. Pat. No. 6,399,336 B1. Therein, in vitro methods for sialylating recombinant glycoproteins are disclosed where a sialic acid donor moiety is combined with a glycoprotein having a galactose or N-acetylgalactosamine acceptor moiety. In such methods a sialyltransferase combined with the acceptor and donor attached a sialic acid to a saccharide.

A recombinant tissue protective cytokine of the invention may have at least a reduced number of N-linked carbohydrates. To remove N-linked carbohydrates, a recombinant tissue protective cytokine may be treated with hydrazine, in accordance, for example, with the methods described by Hermentin et al., 1996, Glycobiology 6(2):217-30. As noted above, erythropoietin has three N-linked carbohydrate moieties; the present invention embraces those erythropoietins with two, one, or no N-linked carbohydrate.

A recombinant tissue protective cytokine of the invention may have at least a reduced carbohydrate content by virtue of treatment of a recombinant tissue protective cytokine with at least one glycosidase. For example, the procedure of Chen and Evangelista, 1998, Electrophoresis 19(15):2639-44, may be followed. Furthermore, removal of the 0-linked carbohydrate may be achieved following the methods described in Hokke et al., 1995, Eur. J Biochem.228(3):981-1008.

The carbohydrate portion of a recombinant tissue protective cytokine molecule may have at least a non-mammalian glycosylation pattern by virtue of the expression of a recombinant erythropoietin mutein in non-mammalian cells. Preferably, the recombinant tissue protective cytokines of the invention are expressed in insect or plant cells. By way of non-limiting example, expression of a recombinant tissue protective cytokine in insect cells using a baculovirus expression system may be carried out in accordance with Quelle et al., 1989, Blood 74(2):652-657. Another method is described in U.S. Pat. No. 5,637,477. Expression in a plant system may be carried out in accordance with the method of Matsumoto et al., 1993, Biosci. Biotech. Biochem. 57(8):1249-1252. Alternatively, expression in bacteria will result in non-glycosylated forms of a recombinant tissue protective cytokine. These are merely exemplary of methods useful for the production of a recombinant tissue protective cytokine of the invention and are in no way limiting.

A non-limiting example of modification of glycosylation patterns is using fucosylation as disclosed in U.S. patent application Ser. No. U.S. 2003/0040037 A1 and in U.S. patent application Ser. No. U.S. 2003/0003529 A1. Therein, methods are disclosed for modifying a glycosylation pattern of a glycopeptide by contacting a glycopeptide having an acceptor moiety for a fucosyltransferase with a reaction mixture having a fucose donor moiety to modify the glycosylation pattern of the glycopeptide. Methods are also disclosed for modification of glycosylation patterns using recombinant glycopeptide.

A recombinant tissue protective cytokine of the invention may have at least one or more oxidized carbohydrates that also may be chemically reduced. For example, the recombinant tissue protective cytokine may be a periodate-oxidized erythropoietin mutein; the periodate-oxidized erythropoietin mutein also may be chemically reduced with a borohydride salt such as sodium borohydride or sodium cyanoborohydride. Periodate oxidation of erythropoietin mutein may be carried out, for example, by the methods described by Linsley et al., 1994, Anal. Biochem. 219(2):207-17. Chemical reduction following periodate oxidation may be carried out following the methods of Tonelli and Meints, 1978, J. Supramol. Struct. 8(l):67-78.

It should be noted that certain of the aforementioned and following amino acid modifications to a native erythropoietin may not be possible as the particular target amino acid for chemical modification in the native molecule has been altered to form the recombinant tissue protective cytokine of the invention. Of course, the altered amino acid may be subject to chemical modification in its own right, and the present invention embraces all such molecules. One of skill in the art will readily determine the available amino acid residues of a recombinant tissue protective cytokine of the invention and modification(s) available thereto.

A recombinant tissue protective cytokine for the aforementioned uses may have at least one or more modified arginine residues. For example, the recombinant tissue protective cytokine may comprise a R-glyoxal moiety on the one or more arginine residues, where R may be an aryl, heteroaryl, lower alkyl, lower alkoxy, or cycloalkyl group, or an alpha-deoxyglycitolyl group. As used herein, the term lower “alkyl” means a straight- or branched-chain saturated aliphatic hydrocarbon group preferably containing 1-6 carbon atoms. Representative of such groups are methyl, ethyl, isopropyl, isobutyl, butyl, pentyl, hexyl and the like. The term “alkoxy” means a lower alkyl group as defined above attached to the remainder of the molecule by oxygen. Examples of alkoxy include methoxy, ethoxy, propoxy, isopropoxy and the like. The term “cycloalkyl” refers to cyclic alkyl groups with three up to about 8 carbons, including for example cyclopropyl, cyclobutyl, cyclohexyl and the like. The term aryl refers to phenyl and naphthyl groups. The term heteroaryl refers to heterocyclic groups containing 4-10 ring members and 1-3 heteroatoms selected from the group consisting of oxygen, nitrogen and sulfur. Examples include but are not limited to isoxazolyl, phenylisoxazolyl, furyl, pyrimidinyl, quinolyl, tetrahydroquinolyl, pyridyl, imidazolyl, pyrrolidinyl, 1,2,4-triazoylyl, thiazolyl, thienyl, and the like. The R group may be substituted, as for example the 2,3,4-trihydroxybutyl group of 3-deoxyglucosone. Typical examples of R-glyoxal compounds are glyoxal, methylglyoxal, 3-deoxyglucosone, and phenylglyoxal. Preferred R-glyoxal compounds are methylglyoxal or phenylglyoxal. An exemplary method for such modification may be found in Werber et al., 1975, Isr. J. Med. Sci. 11(11): 1169-70, using phenylglyoxal.

In a further example, at least one arginine residue may be modified by reaction with a vicinal diketone such as 2,3-butanedione or cyclohexanedione, preferably in ca. 50 millimolar borate buffer at pH 8-9. A procedure for the latter modification with 2,3-butanedione may be carried out in accordance with Riordan, 1973, Biochemistry 12(20): 3915-3923; and that with cyclohexanone according to Patthy et al., 1975, J. Biol. Chem 250(2): 565-9.

A recombinant tissue protective cytokine of the invention may comprise at least one or more modified lysine residues or a modification of the N-terminal amino group of the erythropoietin molecule, such modifications as those resulting from reaction of the lysine residue with an amino-group-modifying agent. In another embodiment, lysine residues may be modified by reaction with glyoxal derivatives, such as reaction with glyoxal, methylglyoxal and 3-deoxyglucosone to form alpha-carboxyalkyl derivatives. Examples are reaction with glyoxal to form carboxymethyllysine as in Glomb and Monnier, 1995, J. Biol. Chem. 270(17):10017-26, or with methylglyoxal to form (1-carboxyethyl)lysine as in Degenhardt et al., 1998, Cell. Mol. Biol. (Noisy-le-grand) 44(7):1139-45. The modified lysine residue further may be chemically reduced. For example, a recombinant tissue protective cytokine may be biotinylated via lysine groups, in which D-biotinoyl-ε-aminocaproic acid-N-hydroxysuccinimide ester was reacted with erythropoietin, followed by removal of unreacted biotin by gel filtration on a Centricon 10 column, as described by Wojchowski and Caslake, 1989, Blood 74(3):952-8. In this paper, the authors use three different methods of biotinylating erythropoietin, any of which may be used for the preparation of the erythropoietins for the uses herein. Biotin may be added to (1) the sialic acid moieties (2) carboxylate groups or (3) amino groups.

In another preferred embodiment, the lysine may be reacted with an aldehyde or reducing sugar to form an imine, which may be stabilized by reduction as with sodium cyanoborohydride to form an N-alkylated lysine such as glucitolyl lysine, or which in the case of reducing sugars may be stabilized by Amadori or Heyns rearrangement to form an alpha-deoxy alpha-amino sugar such as alpha-deoxy-alpha-fructosyllysine. As an example, preparation of a fructosyllysine-modified protein by incubation with 0.5 M glucose in sodium phosphate buffer at pH 7.4 for 60 days is described by Makita et al., 1992, J. Biol. Chem. 267:5133-5138. In another example, the lysine group may be carbamylated, such as by virtue of reaction with cyanate ion, or alkyl- or aryl-carbamylated or -thiocarbamylated with an alkyl- or aryl-isocyanate or -isothiocyanate, or it may be acylated by a reactive alkyl- or arylcarboxylic acid derivative, such as by reaction with acetic anhydride or succinic anhydride or phthalic anhydride. Exemplary are the modification of lysine groups with 4-sulfophenylisothiocyanate or with acetic anhydride, both as described in Gao et al., 1994, Proc Natl Acad Sci USA 91(25):12027-30. Lysine groups may also be trinitrophenyl modified by reaction with trinitrobenzenesulfonic acid or preferably its salts.

At least one tyrosine residue of a recombinant tissue protective cytokine may be modified in an aromatic ring position by an electrophilic reagent, such as by nitration or iodination. By way of non-limiting example, erythropoietin may be reacted with tetranitromethane (Nestler et al., 1985, J. Biol. Chem. 260(12):7316-21; or iodinated as described in Example 4.

At least an aspartic acid or a glutamic acid residue of a recombinant tissue protective cytokine may be modified, such as by reaction with a carbodiimide followed by reaction with an amine such as but not limited to glycinamide.

In another example, a tryptophan residue of a recombinant tissue protective cytokine may be modified, such as by reaction with n-bromosuccinimide or n-chlorosuccinimide, following methods such as described in Josse et al., Chem Biol Interact 1999 May 14;1 19-120.

In yet another example, a recombinant tissue protective cytokine may be prepared by removing at least one amino group, such may be achieved by reaction with ninhydrin followed by reduction of the subsequent carbonyl group by reaction with borohydride.

In still a further example, a recombinant tissue protective cytokine is provided that has at least an opening of at least one of the cysteine linkages in the erythropoietin molecule by reaction with a reducing agent such as dithiothreitol, followed by reaction of the subsequent sulfhydryls with iodoacetamide, iodoacetic acid or another electrophile to prevent reformation of the disulfide linkages. As noted above, alternatively or in combination, disulfide linkages may be abolished by altering a cysteine molecule that participates in the actual cross-link or at least one other amino acid residue that results in the inability of the erythropoietin mutein to form at least one of the disulfide linkages present in the native molecule.

A recombinant tissue protective cytokine may be prepared by subjecting an erythropoietin to a limited chemical proteolysis that targets specific residues, for example, to cleave after tryptophan residues. Such resulting recombinant tissue protective cytokine fragments are embraced herein.

As noted above, a recombinant tissue protective cytokine useful for the purposes herein may have at least one of the aforementioned modifications, but may have more than one of the above modifications. By way of example of a recombinant tissue protective cytokine with one modification to the carbohydrate portion of the molecule and one modification to the amino acid portion, a recombinant tissue protective cytokine may be asialoerythropoietin and have its lysine residue at position 45 changed to aspartic acid.

Thus, various recombinant tissue protective cytokine molecules and pharmaceutical compositions containing them for the uses described herein are embraced. As mentioned above, such erythropoietin molecules include but are not limited to muteins that are further asialoerythropoietin, N-deglycosylated erythropoietin, O-deglycosylated erythropoietin, erythropoietin with reduced carbohydrate content, erythropoietin with altered glycosylation patterns, erythropoietin with carbohydrates oxidized then reduced, arylglyoxal-modified erythropoietin, alkylglyoxal-modified erythropoietin, 2,3-butanedione-modified erythropoietin, cyclohexanedione-modified erythropoietin, biotinylated erythropoietin, N-alkylated-lysyl-erythropoietin, glucitolyl lysine erythropoietin, alpha-deoxy-alpha-fructosyllysine-erythropoietin, carbamylated erythropoietin, acetylated erythropoietin, succinylated erythropoietin, alpha-carboxyalkyl erythropoietin, nitrated erythropoietin, iodinated erythropoietin, to name some representative yet non-limiting examples based on the teachings herein. Preferred are the aforementioned modified forms based on human erythropoietin.

Moreover, the invention encompasses the aforementioned recombinant tissue protective cytokines, and pharmaceutical compositions comprising such compounds. By way of non-limiting example, such recombinant tissue protective cytokines include periodate-oxidized erythropoietin mutein, glucitolyl lysine erythropoietin mutein, fructosyl lysine erythropoietin mutein, 3-deoxyglucosone erythropoietin mutein, and carbamylated asialoerythropoietin mutein.

### 5.3. EXPRESSION SYSTEMS

A variety of host-expression vector systems may be utilized to produce the recombinant tissue protective cytokines, including erythropoietin mutein molecules of the invention. Such host-expression systems represent vehicles by which the recombinant tissue protective cytokines of interest may be produced and subsequently purified, but also represent cells that may, when transformed or transfected with the appropriate nucleotide coding sequences, exhibit the modified erythropoietin gene product in situ. These include, but are not limited to, bacteria, insect, plant, mammalian, including human host systems, such as, but not limited to, insect cell systems infected with recombinant virus expression vectors (e.g., baculovirus) containing the recombinant tissue protective cytokine product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing recombinant tissue protective cytokine coding sequences; or mammalian cell systems, including human cell systems, (e.g., HT1080, COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

An expression construct, as used herein, refers to a nucleotide sequence encoding a recombinant tissue protective cytokine operably associated with one or more regulatory regions which allows expression of the recombinant tissue protective cytokine in an appropriate host cell. “Operably-associated” refers to an association in which the regulatory regions and the recombinant tissue protective cytokine polypeptide sequence to be expressed are joined and positioned in such a way as to permit transcription, and ultimately, translation of the recombinant tissue protective cytokine sequence. A variety of expression vectors may be used for the expression of recombinant tissue protective cytokine, including, but not limited to, plasmids, cosmids, phage, phagemids, or modified viruses. Examples include bacteriophages such as lambda derivatives, or plasmids such as pBR322 or pUC plasmid derivatives or the Bluescript vector (Stratagene). Typically, such expression vectors comprise a functional origin of replication for propagation of the vector in an appropriate host cell, one or more restriction endonuclease sites for insertion of the recombinant tissue protective cytokine gene sequence, and one or more selection markers.

In preferred embodiments, the pCI-neo vector is used to anneal oligonucleotides to the original human EPO cDNA clone to introduce the mutations as described above. The pCI-neo vector contains the neomycin phosphotransferase gene, a selectable marker for mammalian cells. The pCI-neo Vector can be used for transient expression or for stable expression by selecting transfected cells with the antibiotic G-418. (Brondyk, 1995, New Mammalian Expression Vector with a selectable marker: pCI-neo. Promega Notes 51, 10-14).

For expression of recombinant tissue protective cytokine in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include, but are not limited to, those associated with the metallothionein II gene, mouse mammary tumor virus glucocorticoid responsive long terminal repeats (MMTV-LTR), and the α-interferon gene (Williams et al., 1989, Cancer Res. 49: 2735-42 Taylor et al., 1990, Mol. Cell. Biol. 10: 165-75).

The efficiency of expression of the recombinant tissue protective cytokine in a host cell may be enhanced by the inclusion of appropriate transcription enhancer elements in the expression vector, such as those found in SV40 virus, Hepatitis B virus, cytomegalovirus, immunoglobulin genes, metallothionein, a-actin (see Bittner et al., 1987, Methods in Enzymol. 153: 516-544; Gorman, 1990, Curr. Op. in Biotechnol. 1: 36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding a recombinant tissue protective cytokine. For long term, high yield production of recombinant tissue protective cytokines, stable expression in mammalian, plant, bacterial, or ftingal cells can be used. A number of selection systems may be used for mammalian cells, including, but not limited, to the Herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. U.S.A. 48:2026), and adenine phosphoribosyltransferase (Lowy et al., 1980, Cell 22:817) genes can be employed in tk-, hgprt- or aprt- cells, respectively. Also, antimetabolite resistance can be used as the basis of selection for dihydrofolate reductase (dhfr), which confers resistance to methotrexate (Wigler et al., 1980, Natl. Acad. Sci. U.S.A. 77:3567; O'Hare et al., 1981, Proc. Natl. Acad. Sci. U.S.A. 78:1527); gpt, which confers resistance to mycophenolic acid (Mulligan and Berg, 1981, Proc. Natl. Acad. Sci. U.S.A. 78:2072); neomycin phosphotransferase (neo), which confers resistance to the aminoglycoside G-418 (Colberre-Garapin et al., 1981, J. Mol. Biol. 150: 1); and hygromycin phosphotransferase (hyg), which confers resistance to hygromycin (Santerre et al., 1984, Gene 30:147). Other selectable markers, such as but not limited to histidinol and Zeocin™ can also be used.

In order to insert the recombinant tissue protective cytokine coding sequence into the cloning site of a vector, DNA sequences with regulatory functions, such as promoters, must be attached to the coding sequences. To do this, linkers or adapters providing the appropriate compatible restriction sites may be ligated to the ends of cDNA or synthetic DNA encoding a recombinant tissue protective cytokine, by techniques well known in the art (Wu et al., 1987, Methods Enzymol. 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

The expression construct comprising a recombinant tissue protective cytokine-coding sequence operably associated with regulatory regions can be directly introduced into appropriate host cells for expression and production of the recombinant tissue protective cytokines of the invention without further cloning (see e.g., U.S. Pat. No. 5,580,859). The expression constructs may also contain DNA sequences that facilitate integration of the coding sequence into the genome of the host cell, e.g., via homologous recombination. In this instance, it is not necessary to employ an expression vector comprising a replication origin suitable for appropriate host cells in order to propagate and express the recombinant tissue protective cytokines in the host cells.

Expression constructs containing cloned recombinant tissue protective cytokines coding sequences can be introduced into the mammalian host cell by a variety of techniques known in the art, including but not limited to calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc. Natl. Acad. Sci. 84:3344), and microinjection (Cappechi, 1980, Cell 22:479-488).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and WI38.

For long-term, high-yield production of recombinant proteins, stable expression is preferred. For example, cell lines that stably express the recombinant tissue protective cytokine-related molecule gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then they are switched to a selective media. The selectable marker in the recombinant plasmid confers resistance to the selection and allows cells to integrate the plasmid into their chromosomes in a stable manner and grow to form foci that in turn can be cloned and expanded into cell lines. This method may advantageously be used to engineer cell lines that express the recombinant tissue protective cytokine gene product. Such engineered cell lines may be particularly useful in screening and evaluation of compounds that affect the endogenous activity of the recombinant tissue protective cytokine gene product.

Any of the cloning and expression vectors described herein may be synthesized and assembled from known DNA sequences by techniques well known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

Alternatively, a number of viral-based expression systems may also be utilized with mammalian cells for recombinant expression of tissue protective cytokines. Vectors using DNA virus backbones have been derived from simian virus 40 (SV40) (Hamer et al., 1979, Cell 17:725), adenovirus (Van Doren et al., 1984, Mol. Cell Biol. 4:1653), adeno-associated virus (McLaughlin et al., 1988, J. Virol. 62:1963), and bovine papillomas virus (Zinn et al., 1982, Proc. Natl. Acad. Sci. 79:4897). In cases where an adenovirus is used as an expression vector, the donor DNA sequence may be ligated to an adenovirus transcription/translation control region, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts (see, e.g., Logan and Shenk, 1984, Proc. Natl. Acad. Sci. U.S.A. 81:3655-3659).

Alternatively, the vaccinia 7.5K promoter may be used (see, e.g., Mackett et al., 1982, Proc. Natl. Acad. Sci. U.S.A. 79:7415-7419; Mackett et al., 1984, J. Virol. 49:857-864; Panicali et al., 1982, Proc. Natl. Acad. Sci. U.S.A. 79:4927-4931) In cases where a human host cell is used, vectors based on the Epstein-Barr virus (EBV) origin (OriP) and EBV nuclear antigen 1 (EBNA-1; a trans-acting replication factor) may be used. Such vectors can be used with a broad range of human host cells, e.g., EBO-pCD (Spickofsky et al., 1990, DNA Prot. Eng. Tech. 2:14-18), pDR2 and oDR2 (available from Clontech Laboratories).

Recombinant tissue protective cytokine expression can also be achieved by a retrovirus-based expression system. In contrast to transfection, retroviruses can efficiently infect and transfer genes to a wide range of cell types including, for example, primary hematopoietic cells. In retroviruses such as Moloney murine leukemia virus, most of the viral gene sequences can be removed and replaced with a recombinant tissue protective cytokine coding sequence, while the missing viral functions can be supplied in trans. The host range for infection by a retroviral vector can also be manipulated by the choice of envelope used for vector packaging.

For example, a retroviral vector can comprise a 5′ long terminal repeat (LTR), a 3′ LTR, a packaging signal, a bacterial origin of replication, and a selectable marker. The recombinant tissue protective cytokine DNA is inserted into a position between the 5′ LTR and 3′ LTR, such that transcription from the 5′ LTR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells (see McLauchlin et al., 1990, Prog. Nucleic Acid Res. and Molec. Biol. 38:91-135; Morgenstern et al., 1990, Nucleic Acid Res. 18:3587-3596; Choulika et al., 1996, J. Virol 70:1792-1798; Boesen et al., 1994, Biotherapy 6:291-302; Salmons and Gunzberg, 1993, Human Gene Therapy 4:129-141; and Grossman and Wilson, 1993, Curr. Opin. in Genetics and Devel. 3:110-114).

In one embodiment of the invention, a recombinant tissue protective cytokine deficient in sialic residues, or completely lacking sialic residues, may be produced in mammalian cell, including a human cell. Such cells may be engineered to be deficient in, or lacking, the enzymes that add sialic acids, i.e., the β-galactoside a 2,3 sialyltransferase (Aα2,3 sialyltransferase@) and the β-galactoside a 2,6 sialyltransferase (Aα2,6 sialyltransferase@) activity). In one embodiment, a mammalian cell is used in which either or both the α2,3 sialyltransferase gene and/or the α2,6 sialyltransferase gene, is deleted. Such deletions may be constructed using gene knock-out techniques well known in the art. In another embodiment, dihydrofolate reductase (DHFR) deficient Chinese Hamster Ovary (CHO) cells are used as the host cell for the production of recombinant tissue protective cytokines. CHO cells do not express the enzyme α2,6 sialyltransferase and therefore do not add sialic acid in the 2,6 linkage to N-linked oligosaccharides of glycoproteins produced in these cells. As a result, recombinant proteins produced in CHO cells lack sialic acid in the 2,6 linkage to galactose (Sasaki et al. (1987; Takeuchi et al. supra; Mutsaers et al Eur. J Biochem. 156, 651 (1986); Takeuchi et al. J. Chromotgr. 400, 207 (1987). In one embodiment, to produce a host cell for the production of asialo-erythropoietin, the gene encoding α2,3 sialyltransferase in CHO cells is deleted. Such α2,3 sialyltransferase knock-out CHO cells completely lack sialyltransferase activity, and as a result, are useful for the recombinant expression and production of asialoerythropoietin mutein.

In another embodiment, asialo glycoproteins can be produced by interfering with sialic acid transport into the Golgi apparatus e.g., Eckhardt et al., 1998, J. Biol. Chem. 273:20189-95). Using methods well known to those skilled in the art (e.g., Oelmann et al., 2001, J. Biol. Chem. 276:26291-300), mutagenesis of the nucleotide sugar CMP-sialic acid transporter can be accomplished to produce mutants of Chinese hamster ovary cells. These cells cannot add sialic acid residues to glycoproteins such as a recombinant tissue protective cytokine and produce only asialoerythropoietin mutein.

Transfected mammalian cells producing erythropoietin mutein also produce cytosolic sialidase which if it leaks into the culture medium degrades sialoerythropoietin mutein with high efficiency (e.g., Gramer et al, 1995 Biotechnology 13:692-698). Using methods well known to those knowledgeable in the art (e.g., from information provided in Ferrari et al, 1994, Glycobiology 4:367-373), cell lines can be transfected, mutated or otherwise caused to constitutively produce sialidase. In this manner, asialoerythropoietin mutein can be produced during the manufacture of asialoerythropoietin mutein.

The recombinant cells may be cultured under standard conditions of temperature, incubation time, optical density, and media composition. Alternatively, modified culture conditions and media may be used to enhance production of recombinant tissue protective cytokine. For example, recombinant cells may be grown under conditions that promote inducible recombinant tissue protective cytokine expression. Any technique known in the art may be applied to establish the optimal conditions for producing recombinant tissue protective cytokines. Cellular lysates or extracts comprising recombinant tissue protective cytokines can be further purified to isolate recombinant tissue protective cytokines.

To facilitate purification of the recombinant tissue protective cytokines, a marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag. Any purification method known in the art can be used (see e.g., International Patent Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452).

### 5.4. ASSAYS FOR TISSUE PROTECTIVE PROPERTIES OF THE

### RECOMBINANT TISSUE PROTECTIVE CYTOKINES

Following the manufacture of the recombinant tissue protective cytokines and in some embodiments further chemical modification of such tissue protective cytokines of the present invention, one of ordinary skill in the art can verify the tissue protective attributes of the cytokines and the absence of an effect on the bone marrow using well known assays.

For example, the non-erythropoietic affect of a recombinant tissue protective cytokine can be verified through the use of a TF-1 assay. In this assay, TF-1 cells are grown in a complete RPMI medium supplemented with 5 ng/ml of GM-CSF and 10% FCS for a day at 37° C. in a CO2 incubator. The cells are then washed in and suspended at a density of 106 cells/ml for 16 h in starvation medium (5% FCS without GM-CSF). A 96 well plate is prepared by: (1) adding 100 μl of sterile water to the outer wells to maintain moisture; (2) adding medium (10% FCS without cells or GM-CSF) alone to 5 wells; and (3) seeding 25,000 cells/well with medium containing 10% FCS and the recombinant tissue protective cytokines in the remaining cells (five wells per cytokine being tested). If the cells proliferate, the recombinant tissue protective cytokine may be erythropoietic. The in vivo effect of the compound should then be tested on an in vivo assay monitoring the increase of hematocrit due to the recombinant tissue protective cytokine. A negative result—non proliferation of cells in the TF-1 in vitro assay and/or no increase in hematocrit within the in vivo assay—means that the recombinant tissue protective cytokine is nonerythropoietic.

As an alternative to the TF-1 assay described above, one skilled in the art may employ other erythropoietic assays known in the art, including, but not limited to, UT-7 cell assays, such as those described below in the Examples sections.

The tissue protective properties of the recombinant tissue protective cytokines may be verified using a P-19 in vitro assay or a water intoxication in vivo assay in mice, both of which are outlined in further detail below. Alternative assays, include but are not limited to the additional assays outlined in the Examples below, such as the PC-12, and hypocampal slice assays. The above assays are provided merely as examples, and other suitable assays for determining the tissue protective effects and/or bone marrow effects of the recombinant tissue protective cytokines known to those of ordinary skill in the art are contemplated as well.

### 5.5. PHARMACEUTICAL COMPOSITIONS OF THE INVENTION

In the practice of one aspect of the present invention, a pharmaceutical composition as described above containing a recombinant tissue protective cytokine may be administerable to a mammal by any route which provides a sufficient level of a recombinant tissue protective cytokine in the vasculature to permit translocation across an endothelial cell barrier and beneficial effects on responsive cells. When used for the purpose of perfusing a tissue or organ, similar results are desired. In the instance wherein the erythropoietin mutein is used for ex-vivo perfusion, the recombinant tissue protective cytokine may be any form of erythropoietin mutein, such as the aforementioned recombinant tissue protective cytokine. In the instance where the cells or tissue is non-vascularized and/or the administration is by bathing the cells or tissue with the composition of the invention, the pharmaceutical composition provides an effective responsive cell-beneficial amount of a recombinant tissue protective cytokine. The endothelial cell barriers across which a recombinant tissue protective cytokine may translocate include tight junctions, perforated junctions, fenestrated junctions, and any other types of endothelial barriers present in a mammal. A preferred barrier is an endothelial cell tight junction, but the invention is not so limiting.

The aforementioned recombinant tissue protective cytokines are useful generally for the therapeutic or prophylactic treatment of human diseases of the central nervous system or peripheral nervous system which have primarily neurological or psychiatric symptoms, ophthalmic diseases, cardiovascular diseases, cardiopulmonary diseases, respiratory diseases, kidney, urinary and reproductive diseases, gastrointestinal diseases and endocrine and metabolic abnormalities. In particular, such conditions and diseases include hypoxic conditions, which adversely affect excitable tissues, such as excitable tissues in the central nervous system tissue, peripheral nervous system tissue, or cardiac or retinal tissue such as, for example, brain, heart, or retina/eye. Therefore, the invention can be used to treat or prevent damage to excitable tissue resulting from hypoxic conditions in a variety of conditions and circumstances. Non-limiting examples of such conditions and circumstances are provided in the table hereinbelow.

In the example of the protection of neuronal tissue pathologies treatable in accordance with the present invention, such pathologies include those which result from reduced oxygenation of neuronal tissues. Any condition which reduces the availability of oxygen to neuronal tissue, resulting in stress, damage, and finally, neuronal cell death, can be treated by the methods of the present invention. Generally referred to as hypoxia and/or ischemia, these conditions arise from or include, but are not limited to, stroke, vascular occlusion, prenatal or postnatal oxygen deprivation, suffocation, choking, near drowning, carbon monoxide poisoning, smoke inhalation, trauma, including surgery and radiotherapy, asphyxia, epilepsy, hypoglycemia, chronic obstructive pulmonary disease, emphysema, adult respiratory distress syndrome, hypotensive shock, septic shock, anaphylactic shock, insulin shock, sickle cell crisis, cardiac arrest, dysrhythmia, nitrogen narcosis, and neurological deficits caused by heart-lung bypass procedures.

In one embodiment, for example, the specific recombinant tissue protective cytokine compositions can be administered to prevent injury or tissue damage resulting from risk of injury or tissue damage during surgical procedures, such as, for example, tumor resection or aneurysm repair. Other pathologies caused by or resulting from hypoglycemia which are treatable by the methods described herein include insulin overdose, also referred to as iatrogenic hyperinsulinemia, insulinoma, growth hormone deficiency, hypocortisolism, drug overdose, and certain tumors.

Other pathologies resulting from excitable neuronal tissue damage include seizure disorders, such as epilepsy, convulsions, or chronic seizure disorders. Other treatable conditions and diseases include, but are not limited to, diseases such as stroke, multiple sclerosis, hypotension, cardiac arrest, Alzheimer's disease, Parkinson's disease, cerebral palsy, brain or spinal cord trauma, AIDS dementia, age-related loss of cognitive function, memory loss, amyotrophic lateral sclerosis, seizure disorders, alcoholism, retinal ischemia, optic nerve damage resulting from glaucoma, and neuronal loss.

The specific composition and methods of the present invention may be used to treat inflammation resulting from disease conditions or various traumas, such as physically or chemically induced inflammation. Such traumas could include angitis, chronic bronchitis, pancreatitis, osteomyelitis, rheumatoid arthritis, glomerulonephritis, optic neuritis, temporal arteritis, encephalitis, meningitis, transverse myelitis, dermatomyositis, polymyositis, necrotizing fascilitis, hepatitis, and necrotizing enterocolitis.

Evidence has demonstrated that activated astrocytes can exert a cytotoxic role towards neurons by producing neurotoxins. Nitric oxide, reactive oxygen species, and cytokines are released from glial cells in response to cerebral ischemia (see Becker, K. J. 2001. Targeting the central nervous system inflammatory response in ischemic stroke. Curr Opinion Neurol 14:349-353 and Mattson, M. P., Culmsee, C., and Yu, Z. F. 2000. Apoptotic and Antiapoptotic mechanisms in stroke. Cell TissueRes 301:173-187.). Studies have further demonstrated that in models of neurodegeneration, glial activation and subsequent production of inflammatory cytokines depends upon primary neuronal damage (see Viviani, B., Corsini, E., Galli, C. L., Padovani, A., Ciusani, E., and Marinovich, M. 2000. Dying neural cells activate glia through the release of a protease product. Glia 32:84-90 and Rabuffetti, M., Scioratti, C., Tarozzo, G., Clementi, E., Manfredi, A. A., and Beltramo, M. 2000. Inhibition of caspase-1-like activity by Ac-Tyr-Val-Ala-Asp-chloromethyl ketone includes long lasting neuroprotection in cerebral ischemia through apoptosis reduction and decrease of proinflammatory cytokines. J Neurosci 20:4398-4404). Inflammation and glial activation is common to different forms of neuro degenerative disorders, including cerebral ischemia, brain trauma and experimental allergic encephalomyelitis, disorders in which erythropoietin exerts a cellular protective effect. Inhibition of cytokine production by erythropoietin could, at least in part, mediate its protective effect. However, unlike “classical” anti-inflammatory cytokines such as I1-10 and IL-13, which inhibit tumor necrosis factor production directly, erythropoietin appears to be active only in the presence of neuronal death.

While not wishing to be bound by any particular theory, it appears that this anti-inflammatory activity may be hypothetically explained by several non-limiting theories. First, since erythropoietin prevents apoptosis, inflammatory events triggered by apoptosis would be prevented. Additionally, erythropoietin may prevent the release of molecular signals from dying neurons which stimulate the glia cells or could act directly on the glial cells reducing their reaction to these products. Another possibility is that erythropoietin targets more proximal members of the inflammatory cascade (e.g., caspase 1, reactive oxygen or nitrogen intermediates) that trigger both apoptosis and inflammation.

Furthermore, erythropoietin appears to provide anti-inflammatory protection without the rebound affect typically associated with other anti-inflammatory compounds such as dexamethasone. Once again, not wishing to be bound by any particular theory, it appears as though this may be due to erythropoietin's affect on multipurpose neuro toxins such as nitric oxide (NO). Although activated astrocytes and microglia produce neurotoxic quantities of NO in response to various traumas, NO serves many purposes within the body including the modulation of essential physiological functions. Thus, although the use of an anti-inflammatory may alleviate inflammation by suppressing NO or other neuro toxins, if the anti-inflammatory has too long a half-life it may also interfere with these chemicals' roles in repairing the damage resulting from the trauma that led to the inflammation. It is hypothesized that the recombinant tissue protective cytokines of the present invention are able to alleviate the inflammation without interfering with the restorative capabilities of neurotoxins such as NO.

The specific compositions and methods of the invention may be used to treat conditions of, and damage to, retinal tissue. Such disorders include, but are not limited to retinal ischemia, macular degeneration, retinal detachment, retinitis pigmentosa, arteriosclerotic retinopathy, hypertensive retinopathy, retinal artery blockage, retinal vein blockage, hypotension, and diabetic retinopathy.

In another embodiment, the methods principles of the invention may be used to protect or treat injury resulting from radiation damage to excitable tissue. A further utility of the methods of the present invention is in the treatment of neurotoxin poisoning, such as domoic acid shellfish poisoning, neurolathyrism, and Guam disease, amyotrophic lateral sclerosis, and Parkinson's disease.

As mentioned above, the present invention is also directed to a method for enhancing excitable tissue function in a mammal by peripheral administration of a recombinant tissue protective cytokine as described above. Various diseases and conditions are amenable to treatment using this method, and further, this method is useful for enhancing cognitive function in the absence of any condition or disease. These uses of the present invention are described in further detail below and include enhancement of learning and training in both human and non-human mammals.

Conditions and diseases treatable by the methods of this aspect of the present invention directed to the central nervous system include, but are not limited to, mood disorders, anxiety disorders, depression, autism, attention deficit hyperactivity disorder, and cognitive dysfunction. These conditions benefit from enhancement of neuronal function. Other disorders treatable in accordance with the teachings of the present invention include for example, sleep disruption, sleep apnea, and travel-related disorders; subarachnoid and aneurismal bleeds, hypotensive shock, concussive injury, septic shock, anaphylactic shock, and sequelae of various encephalitides and meningitides, for example, connective tissue disease-related cerebritides such as lupus. Other uses include prevention of or protection from poisoning by neurotoxins, such as domoic acid shellfish poisoning, neurolathyrism, and Guam disease, amyotrophic lateral sclerosis, Parkinson's disease; postoperative treatment for embolic or ischemic injury; whole brain irradiation; sickle cell crisis; and eclampsia.

A further group of conditions treatable by the methods of the present invention include mitochondrial dysfunction, of either a hereditary or an acquired nature, which are the cause of a variety of neurological diseases typified by neuronal injury and death. For example, Leigh disease (subacute necrotizing encephalopathy) is characterized by progressive visual loss and encephalopathy, due to neuronal drop out, and myopathy. In these cases, defective mitochondrial metabolism fails to supply enough high energy substrates to fuel the metabolism of excitable cells. An erythropoietin receptor activity modulator optimizes failing function in a variety of mitochondrial diseases. As mentioned above, hypoxic conditions adversely affect excitable tissues. The excitable tissues include, but are not limited to, central nervous system tissue, peripheral nervous system tissue, and heart tissue. In addition to the conditions described above, the methods of the present invention are useful in the treatment of inhalation poisoning, such as carbon monoxide and smoke inhalation, severe asthma, adult respiratory distress syndrome, choking, and near drowning. Further conditions which create hypoxic conditions or by other means induce excitable tissue damage include hypoglycemia that may occur in inappropriate dosing of insulin, or with insulin-producing neoplasms (insulinoma).

Various neuropsychologic disorders which are believed to originate from excitable tissue damage are treatable by the instant methods. Chronic disorders in which neuronal damage is involved and for which treatment by the present invention is provided include disorders relating to the central nervous system and/or peripheral nervous system including age-related loss of cognitive flinction and senile dementia, chronic seizure disorders, Alzheimer's disease, Parkinson's disease, dementia, memory loss, amyotrophic lateral sclerosis, multiple sclerosis, tuberous sclerosis, Wilson's Disease cerebral and progressive supranuclear palsy, Guam disease, Lewy body dementia, prion diseases, such as spongiform encephalopathies, e.g., Creutzfeldt-Jakob disease, Huntington's disease, myotonic dystrophy, Freidrich's ataxia and other ataxias, as well as Gilles de la Tourette's syndrome, seizure disorders such as epilepsy and chronic seizure disorder, stroke, brain or spinal cord trauma, AIDS dementia, alcoholism, autism, retinal ischemia, glaucoma, autonomic function disorders such as hypertension and sleep disorders, and neuropsychiatric disorders that include, but are not limited to, schizophrenia, schizoaffective disorder, attention deficit disorder hyperactivity, dysthymic disorder, major depressive disorder, mania, obsessive-compulsive disorder, psychoactive substance use disorders, anxiety, panic disorder, as well as unipolar and bipolar affective disorders. Additional neuropsychiatric and neurodegenerative disorders include, for example, those listed in the American Psychiatric Association's Diagnostic and Statistical Manual of Mental Disorders (DSM), the most current version, IV, of which in incorporated herein by reference in its entirety.

In another embodiment, recombinant chimeric toxin molecules comprising a recombinant tissue protective cytokine can be used for therapeutic delivery of toxins to treat a proliferative disorder, such as cancer, or viral disorder, such as subacute sclerosing panencephalitis.

The following table lists additional exemplary, non-limiting indications as to the various conditions and diseases amenable to treatment by the aforementioned recombinant tissue protective cytokines.

| Cell, tissue or | Dysfunction or | | |
|---|---|---|---|
| organ | pathology | Condition or disease | Type |
| Heart | Ischemia | Coronary artery disease | Acute, chronic |
| | | | Stable, unstable |
| | | Myocardial infarction | Dressler's syndrome |
| | | Angina | |
| | | Congenital heart disease | Valvular |
| | | | Cardiomyopathy |
| | | Prinzmetal angina | |
| | | Cardiac rupture | Aneurysmatic |
| | | | Septal perforation |
| | | Angiitis | |
| | Arrhythmia | Tachy-, bradyarrhythmia | Stable, unstable |
| | | Supraventricular, | Hypersensitive carotid sinus |
| | | ventricular | node |
| | | Conduction abnormalities | |
| | Congestive heart failure | Left, right, bi-ventricular, | Cardiomyopathies, such as |
| | | systolic, diastolic | idiopathic familial, infective, |
| | | | metabolic, storage disease, |
| | | | deficiencies, connective tissue |
| | | | disorder, infiltration and |
| | | | granulomas, neurovascular |
| | | Myocarditis | Autoimmune, infective, |
| | | | idiopathic |
| | | Cor pulmonale | |
| | Blunt and penetrating | | |
| | trauma | | |
| | Toxins | Cocaine toxicity | |
| Vascular | Hypertension | Primary, secondary | |
| | Decompression sickness | | |
| | Fibromuscular | | |
| | hyperplasia | | |
| | Aneurysm | Dissecting, ruptured, | |
| | | enlarging | |
| Lungs | Obstructive | Asthma | |
| | | Chronic bronchitis, | |
| | | Emphysema and airway | |
| | | obstruction | |
| | Ischemic lung disease | Pulmonary embolism, | |
| | | Pulmonary thrombosis, | |
| | | Fat embolism | |
| | Environmental lung | | |
| | diseases | | |
| | Ischemic lung disease | Pulmonary embolism | |
| | | Pulmonary thrombosis | |
| | Interstitial lung disease | Idiopathic pulmonary | |
| | | fibrosis | |
| | Congenital | Cystic fibrosis | |
| | Cor pulmonale | | |
| | Trauma | | |
| | Pneumonia and | Infectious, parasitic, | |
| | pneumonitides | toxic, traumatic, burn, | |
| | | aspiration | |
| | Sarcoidosis | | |
| Pancreas | Endocrine | Diabetes mellitus, type I | Beta cell failure, dysfunction |
| | | and II | Diabetic neuropathy |
| | | Other endocrine cell | |
| | | failure of the pancreas | |
| | Exocrine | Exocrine pancreas failure | pancreatitis |
| Bone | Osteopenia | Primary | Hypogonadism |
| | | secondary | immobilisation |
| | | | Postmenopausal |
| | | | Age-related |
| | | | Hyperparathyroidism |
| | | | Hyperthyroidism |
| | | | Calcium, magnesium, |
| | | | phosphorus and/or vitamin D |
| | | | deficiency |
| | Osteomyelitis | | |
| | Avascular necrosis | | |
| | Trauma | | |
| | Paget's disease | | |
| Skin | Alopecia | Areata | Primary |
| | | Totalis | Secondary |
| | | | Male pattern baldness |
| | Vitiligo | Localized | Primary |
| | | generalized | secondary |
| | Diabetic ulceration | | |
| | Peripheral vascular | | |
| | disease | | |
| | Burn injuries | | |
| Autoimmune | Lupus erythematodes, | | |
| disorders | Sjiogren, | | |
| | Rheumatoid arthritis, | | |
| | Glomerulonephritis, | | |
| | Angiitis | | |
| | Langerhan's histiocytosis | | |
| Eye | Optic neuritis | | |
| | Blunt and penetrating | | |
| | injuries, Infections, | | |
| | Sarcoid, Sickle C disease, | | |
| | Retinal detachment, | | |
| | Temporal arteritis | | |
| | Retinal ischemia, | | |
| | Macular degeneration, | | |
| | Retinitis pigmentosa, | | |
| | Arteriosclerotic | | |
| | retinopathy, Hypertensive | | |
| | retinopathy, Retinal | | |
| | artery blockage, Retinal | | |
| | vein blockage, | | |
| | Hypotension, Diabetic | | |
| | retinopathy, and | | |
| | Macular edema | | |
| Embryonic and | Asphyxia | | |
| fetal disorders | Ischemia | | |
| CNS | Chronic fatigue | | |
| | syndrome, acute and | | |
| | chronic hypoosmolar and | | |
| | hyperosmolar syndromes, | | |
| | AIDS Dementia, | | |
| | Electrocution | | |
| | Encephalitis | Rabies, Herpes | |
| | Meningitis | | |
| | Subdural hematoma | | |
| | Nicotine addiction | | |
| | Drug abuse and | Cocaine, heroin, crack, | |
| | withdrawal | marijuana, LSD, PCP, | |
| | | poly-drug abuse, ecstasy, | |
| | | opioids, sedative | |
| | | hypnotics, | |
| | | amphetamines, caffeine | |
| | Obsessive-compulsive | | |
| | disorders | | |
| | Spinal stenosis, | | |
| | Transverse myelitis, | | |
| | Guillian Barre, Trauma, | | |
| | Nerve root compression, | | |
| | Tumoral compression, | | |
| | Heat stroke | | |
| ENT | Tinnitus | | |
| | Meuniere's syndrome | | |
| | Hearing loss | | |
| | Traumatic injury, | | |
| | barotraumas | | |
| Kidney | Renal failure | Acute, chronic | Vascular/ischemic, interstitial |
| | | | disease, diabetic kidney disease, |
| | | | nephrotic syndromes, infections, |
| | | | injury, contrast-induced, |
| | | | chemotherapy-induced, CPB- |
| | | | induced, or preventive |
| | Henoch S. Purpura | | |
| Striated muscle | Autoimmune disorders | Myasthenia gravis | |
| | | Dermatomyositis | |
| | | Polymyositis | |
| | Myopathies | Inherited metabolic, | |
| | | endocrine and toxic | |
| | Heat stroke | | |
| | Crush injury | | |
| | Rhabdomylosis | | |
| | Mitochondrial disease | | |
| | Infection | Necrotizing fasciitis | |
| Sexual | Central and peripheral | Impotence secondary to | |
| dysfunction | (e.g. erectile dysfunction) | medication, (diabetes) | |
| Liver | Hepatitis | Viral, bacterial, parasitic | |
| | Ischemic disease | | |
| | Cirrhosis, fatty liver | | |
| | Infiltrative/metabolic | | |
| | diseases | | |
| Gastrointestinal | Ischemic bowel disease | | |
| | Inflammatory bowel | | |
| | disease | | |
| | Necrotizing enterocolitis | | |
| Organ | Treatment of donor and | | |
| transplantation | recipient | | |
| Reproductive | Infertility | Vascular | |
| tract | | Autoimmune | |
| | | Uterine abnormalities | |
| | | Implantation disorders | |
| Endocrine | Glandular hyper- and | | |
| | hypofunction | | |

As mentioned above, these diseases, disorders or conditions are merely illustrative of the range of benefits provided by the recombinant tissue protective cytokines of the invention. Accordingly, this invention generally provides therapeutic or prophylactic treatment of the consequences of mechanical trauma or of human diseases. Therapeutic or prophylactic treatment for diseases, disorders or conditions of the CNS and/or peripheral nervous system are preferred. Therapeutic or prophylactic treatment for diseases, disorders or conditions which have a psychiatric component is provided. Therapeutic or prophylactic treatment for diseases, disorders or conditions including, but not limited to, those having an ophthalmic, cardiovascular, cardiopulmonary, respiratory, kidney, urinary, reproductive, gastrointestinal, endocrine, or metabolic component is provided.

In one embodiment, such a pharmaceutical composition of a recombinant tissue protective cytokine may be administered systemically to protect or enhance the target cells, tissue, or organ. Such administration may be parenterally, via inhalation, or transmucosally, e.g., orally, nasally, rectally, intravaginally, sublingually, submucosally or transdermally. Preferably, administration is parenteral, e.g., via intravenous or intraperitoneal injection, and also including, but is not limited to, intra-arterial, intramuscular, intradermal and subcutaneous administration.

For other routes of administration, such as by use of a perfusate, injection into an organ, or other local administration, a pharmaceutical composition will be provided which results in similar levels of a recombinant tissue protective cytokine as described above. A level of about 0.01 pM -30 nM is preferred.

The pharmaceutical compositions of the invention may comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols, and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles, and mouthwashes.

Pharmaceutical compositions adapted for nasal and pulmonary administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. Alternatively, inhalation directly into the lungs may be accomplished by inhalation deeply or installation through a mouthpiece into the oropharynx. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered into the nasal cavity directly or into the lungs via the nasal cavity or oropharynx.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats, and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules, and tablets. In one embodiment, an autoinjector comprising an injectable solution of a recombinant tissue protective cytokine may be provided for emergency use by ambulances, emergency rooms, and battlefield situations, and even for self-administration in a domestic setting, particularly where the possibility of traumatic amputation may occur, such as by imprudent use of a lawn mower. The likelihood that cells and tissues in a severed foot or toe will survive after reattachment may be increased by administering a recombinant tissue protective cytokine to multiple sites in the severed part as soon as practicable, even before the arrival of medical personnel on site, or arrival of the afflicted individual with severed toe at the emergency room.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

Suppositories generally contain active ingredient in the range of 0.5% to 10% by weight; oral formulations preferably contain 10% to 95% active ingredient.

A perfusate composition may be provided for use in transplanted organ baths, for in situ perfusion, or for administration to the vasculature of an organ donor prior to organ harvesting. Such pharmaceutical compositions may comprise levels of a recombinant tissue protective cytokine or a form of a recombinant tissue protective cytokine not suitable for acute or chronic, local or systemic administration to an individual, but will serve the functions intended herein in a cadaver, organ bath, organ perfusate, or in situ perfusate prior to removing or reducing the levels of the recombinant tissue protective cytokine contained therein before exposing or returning the treated organ or tissue to regular circulation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

In another embodiment, for example, a recombinant tissue protective cytokine can be delivered in a controlled-release system. For example, the polypeptide may be administered using intravenous infusion, an implantable osmotic pump, a transdermal patch, liposomes, or other modes of administration. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, the compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); WO 91/04014; U.S. Pat. No. 4,704,355; Lopez-Berestein, ibid., pp. 317-327; see generally ibid.). In another embodiment, polymeric materials can be used (see Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, J. Macromol. Sci. Rev. Macromol. Chem. 23:61, 1953; see also Levy et al., 1985, Science 228:190; During et al., 1989, Ann. Neurol. 25:351; Howard etal., 1989, J. Neurosurg. 71:105).

In yet another embodiment, a controlled release system can be placed in proximity of the therapeutic target, i.e., the target cells, tissue or organ, thus requiring only a fraction of the systemic dose (see, e.g., Goodson, pp. 115-138 in Medical Applications of Controlled Release, vol. 2, supra, 1984). Other controlled release systems are discussed in the review by Langer (1990, Science 249:1527-1533).

In another embodiment, a recombinant tissue protective cytokine, as properly formulated, can be administered by nasal, oral, rectal, vaginal, or sublingual administration.

In a specific embodiment, it may be desirable to administer the recombinant tissue protective cytokine compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

Selection of the preferred effective dose will be determined by a skilled artisan based upon considering several factors which will be known to one of ordinary skill in the art. Such factors include the particular form of recombinant tissue protective cytokine, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, and according to standard clinical techniques.

In another aspect of the invention, a perfusate or perfusion solution is provided for perfusion and storage of organs for transplant, the perfusion solution including an amount of a recombinant tissue protective cytokine effective to protect responsive cells and associated cells, tissues, or organs. Transplant includes, but is not limited to, xenotransplantation, where a organ (including cells, tissue or other bodily part) is harvested from one donor and transplanted into a different recipient; and autotransplant, where the organ is taken from one part of a body and replaced at another, including bench surgical procedures, in which an organ may be removed, and while ex vivo, resected, repaired, or otherwise manipulated, such as for tumor removal, and then returned to the original location. In one embodiment, the perfusion solution is the University of Wisconsin (UW) solution (U.S. Pat. No. 4,798,824) which contains from about 1 to about 25 U/ml erythropoietin, 5% hydroxyethyl starch (having a molecular weight of from about 200,000 to about 300,000 and substantially free of ethylene glycol, ethylene chlorohydrin, sodium chloride and acetone); 25 mM KH2PO4; 3 mM glutathione; 5 mM adenosine; 10 mM glucose; 10 mM HEPES buffer; 5 mM magnesium gluconate; 1.5 mM CaCl2; 105 mM sodium gluconate; 200,000 units penicillin; 40 units insulin; 16 mg Dexamethasone; 12 mg Phenol Red; and has a pH of 7.4-7.5 and an osmolality of about 320 mOSm/l. The solution is used to maintain cadaveric kidneys and pancreases prior to transplant. Using the solution, preservation can be extended beyond the 30-hour limit recommended for cadaveric kidney preservation. This particular perfusate is merely illustrative of a number of such solutions that can be adapted for the present use by inclusion of an effective amount of a recombinant tissue protective cytokine. In a further embodiment, the perfusate solution contains from about 0.01 pg/ml to about 400 ng/ml recombinant tissue protective cytokine, or from about 40 to about 300 ng/ml recombinant tissue protective cytokine. As mentioned above, any form of recombinant tissue protective cytokine can be used in this aspect of the invention.

While the preferred recipient of a recombinant tissue protective cytokine for the purposes herein throughout is a human, the methods herein apply equally to other mammals, particularly domesticated animals, livestock, companion and zoo animals. However, the invention is not so limiting and the benefits can be applied to any mammal.

### 5.6. THERAPEUTIC AND PREVENTATIVE USES OF RECOMBINANT TISSUE PROTECTIVE CYTOKINES

As noted in Example 1 below, the presence of erythropoietin receptors in the brain capillary human endothelium indicates that the targets of the recombinant tissue protective cytokines of the invention are present in the human brain, and that the animal studies on these recombinant tissue protective cytokines of the invention are directly translatable to the treatment or prophylaxis of human beings.

In another aspect of the invention, methods and compositions for enhancing the viability of cells, tissues, or organs which are not isolated from the vasculature by an endothelial cell barrier are provided by exposing the cells, tissue or organs directly to a pharmaceutical composition comprising a recombinant tissue protective cytokine, or administering or contacting an recombinant tissue protective cytokine-containing pharmaceutical composition to the vasculature of the tissue or organ. Enhanced activity of responsive cells in the treated tissue or organ is responsible for the positive effects exerted.

As described above, the invention is based, in part, on the discovery that erythropoietin molecules can be transported from the luminal surface to the basement membrane surface of endothelial cells of the capillaries of organs with endothelial cell tight junctions, including, for example, the brain, retina, and testis. Thus, responsive cells across the barrier are susceptible targets for the beneficial effects of a recombinant tissue protective cytokine, and others cell types or tissues or organs that contain and depend in whole or in part on responsive cells therein are targets for the methods of the invention. While not wishing to be bound by any particular theory, after transcytosis of a recombinant tissue protective cytokine, the recombinant tissue protective cytokine can interact with an erythropoietin receptor on an responsive cell, for example, neuronal, retinal, muscle, heart, lung, liver, kidney, small intestine, adrenal cortex, adrenal medulla, capillary endothelial, testes, ovary, pancreas, bone, skin, or endometrial cell, and receptor binding can initiate a signal transduction cascade resulting in the activation of a gene expression program within the responsive cell or tissue, resulting in the protection of the cell or tissue, or organ, from damage, such as by toxins, chemotherapeutic agents, radiation therapy, hypoxia, etc. Thus, methods for protecting responsive cell-containing tissue from injury or hypoxic stress, and enhancing the function of such tissue are described in detail herein below. As noted above, the methods of the invention are equally applicable to humans as well as to other animals.

In the practice of one embodiment of the invention, a mammalian patient is undergoing systemic chemotherapy for cancer treatment, including radiation therapy, which commonly has adverse effects such as nerve, lung, heart, ovarian, or testicular damage. Administration of a pharmaceutical composition comprising a recombinant tissue protective cytokine as described above is performed prior to and during chemotherapy and/or radiation therapy, to protect various tissues and organs from damage by the chemotherapeutic agent, such as to protect the testes. Treatment may be continued until circulating levels of the chemotherapeutic agent have fallen below a level of potential danger to the mammalian body.

In the practice of another embodiment of the invention, various organs were planned to be harvested from a victim of an automobile accident for transplant into a number of recipients, some of which required transport for an extended distance and period of time. Prior to organ harvesting, the victim was infused with a pharmaceutical composition comprising a recombinant tissue protective cytokine as described herein. Harvested organs for shipment were perfused with a perfusate containing a recombinant tissue protective cytokine as described herein, and stored in a bath comprising recombinant tissue protective cytokine. Certain organs were continuously perfused with a pulsatile perfusion device, utilizing a perfusate containing a recombinant tissue protective cytokine in accordance with the present invention. Minimal deterioration of organ function occurred during the transport and upon implant and reperfusion of the organs in situ.

In another embodiment of the invention, a surgical procedure to repair a heart valve required temporary cardioplegia and arterial occlusion. Prior to surgery, the patient was infused with 4 μg recombinant tissue protective cytokine per kg body weight. Such treatment prevented hypoxic ischemic cellular damage, particularly after reperfusion.

In another embodiment of the invention, in any surgical procedure, such as in cardiopulmonary bypass surgery, a recombinant tissue protective cytokine of the invention can be used. In one embodiment, administration of a pharmaceutical composition comprising a recombinant tissue protective cytokine as described above is performed prior to, during, and/or following the bypass procedure, to protect the function of brain, heart, and other organs.

In the foregoing examples in which a recombinant tissue protective cytokine of the invention is used for ex-vivo applications, or to treat responsive cells such as neuronal tissue, retinal tissue, heart, lung, liver, kidney, small intestine, adrenal cortex, adrenal medulla, capillary endothelial, testes, ovary, or endometrial cells or tissue, the invention provides a pharmaceutical composition in dosage unit form adapted for protection or enhancement of responsive cells, tissues, or organs distal to the vasculature which comprises, per dosage unit, an effective non-toxic amount within the range from about 0.01 pg to 5 mg, 1 pg to 5 mg, 500 pg to 5 mg, 1 ng to 5 mg, 500 ng to 5 mg, 1 μg to 5 mg, 500 μg to 5 mg, or 1 mg to 5 mg of a recombinant tissue protective cytokine and a pharmaceutically acceptable carrier. In a preferred embodiment, the amount of recombinant tissue protective cytokine is within the range from about 1 ng to 5 mg. In a preferred embodiment, the recombinant tissue protective cytokine of the aforementioned composition is non-erythropoietic.

In a further aspect of the invention, EPO administration was found to restore cognitive function in animals having undergone brain trauma. Recombinant tissue protective cytokines of the invention would be expected to have the same cellular protective effects as EPO. After a delay of either 5 days or 30 days, EPO was still able to restore function as compared to sham-treated animals, indicating the ability of a EPO to regenerate or restore brain activity. Thus, the invention is also directed to the use of a recombinant tissue protective cytokine for the preparation of a pharmaceutical composition for the treatment of brain trauma and other cognitive dysfunctions, including treatment well after the injury (e.g. three days, five days, a week, a month, or longer). The invention is also directed to a method for the treatment of cognitive dysfunction following injury by administering an effective amount of a recombinant tissue protective cytokine. Any recombinant tissue protective cytokine as described herein may be used for this aspect of the invention.

Furthermore, this restorative aspect of the invention is directed to the use of any of the recombinant tissue protective cytokines herein for the preparation of a pharmaceutical composition for the restoration of cellular, tissue, or organ dysfunction, wherein treatment is initiated after, and well after, the initial insult responsible for the dysfunction. Moreover, treatment using recombinant tissue protective cytokines of the invention can span the course of the disease or condition during the acute phase as well as a chronic phase.

In the instance wherein a recombinant tissue protective cytokine of the invention has erythropoietic activity, in a preferred embodiment, recombinant tissue protective cytokine may be administered systemically at a dosage between about 0.01 pg and about 100 μg/kg body weight, preferably about 1-50 μg/kg-body weight, most preferably about 5-30 μg/kg-body weight, per administration. This effective dose should be sufficient to achieve serum levels of recombinant tissue protective cytokine greater than about 10,000, 15,000, or 20,000 mU/ml of serum after recombinant tissue protective cytokine administration. Such serum levels may be achieved at about 1, 2, 3, 4, 5, 6, 7, 8, 9, or 10 hours post-administration. Such dosages may be repeated as necessary. For example, administration may be repeated daily, as long as clinically necessary, or after an appropriate interval, e.g., every 1 to 12 weeks, but preferably, every 1 to 3 weeks. In one embodiment, the effective amount of recombinant tissue protective cytokine and a pharmaceutically acceptable carrier may be packaged in a single dose vial or other container. In another embodiment, a recombinant tissue protective cytokine useful for the purposes herein is nonerythropoietic, i.e., it is capable of exerting the activities described herein without causing an increase in hemoglobin concentration or hematocrit. Such a non-erythropoietic form of a recombinant tissue protective cytokine is preferred in instances wherein the methods of the present invention are intended to be provided chronically. In another embodiment, a recombinant tissue protective cytokine is given at a dose greater than that necessary to maximally stimulate erythropoiesis. As noted above, a recombinant tissue protective cytokine of the invention does not necessarily have erythropoietic activity, and therefore the above dosages expressed in units are merely exemplary for recombinant tissue protective cytokines; herein above molar equivalents for dosages are provided which are applicable to any recombinant tissue protective cytokine.

The present invention is further directed to a method for facilitating the transport of a molecule across an endothelial cell barrier in a mammal by administering a composition which comprises the particular molecule in association with a recombinant tissue protective cytokine as described herein above. As described above, tight junctions between endothelial cells in certain organs in the body create a barrier to the entry of certain molecules. For treatment of various conditions within the barriered organ, means for facilitating passage of pharmaceutical agents is desired. A recombinant tissue protective cytokine of the invention is useful as a carrier for delivering other molecules across the blood-brain and other similar barriers. A composition comprising a molecule desirous of crossing the barrier with a recombinant tissue protective cytokine is prepared, and peripheral administration of the composition results in the transcytosis of the composition across the barrier. The association between the molecule to be transported across the barrier and the recombinant tissue protective cytokine may be a labile covalent bond, in which case the molecule is released from association with the recombinant tissue protective cytokine after crossing the barrier. If the desired pharmacological activity of the molecule is maintained or unaffected by association with the recombinant tissue protective cytokine, such a complex can be administered.

The skilled artisan will be aware of various means for associating molecules with a recombinant tissue protective cytokine of the invention and the other agents described above, by covalent, non-covalent, and other means; furthermore, evaluation of the efficacy of the composition can be readily determined in an experimental system. Association of molecules with a recombinant tissue protective cytokine may be achieved by any number of means, including labile, covalent binding, cross-linking, etc. Biotin/avidin interactions may be employed. As mentioned above, a hybrid molecule may be prepared by recombinant or synthetic means, for example, which includes both the domain of the molecule with desired pharmacological activity and the domain responsible for erythropoietin receptor activity modulation.

A molecule may be conjugated to a recombinant tissue protective cytokine through a polyfunctional molecule, i.e., a polyfunctional crosslinker. As used herein, the term “polyfunctional molecule” encompasses molecules having one functional group that can react more than one time in succession, such as formaldehyde, as well as molecules with more than one reactive group. As used herein, the term “reactive group” refers to a functional group on the crosslinker that reacts with a functional group on a molecule (e.g., peptide, protein, carbohydrate, nucleic acid, particularly a hormone, antibiotic, or anti-cancer agent to be delivered across an endothelial cell barrier) so as to form a covalent bond between the cross-linker and that molecule. The term “functional group” retains its standard meaning in organic chemistry. The polyfunctional molecules which can be used are preferably biocompatible linkers, i.e., they are noncarcinogenic, nontoxic, and substantially non-immunogenic in vivo. Polyfunctional cross-linkers such as those known in the art and described herein can be readily tested in animal models to determine their biocompatibility. The polyfunctional molecule is preferably bifunctional. As used herein, the term “bifunctional molecule” refers to a molecule with two reactive groups. The bifunctional molecule may be heterobifunctional or homobifunctional. A heterobiifunctional cross-linker allows for vectorial conjugation. It is particularly preferred for the polyfunctional molecule to be sufficiently soluble in water for the cross-linking reactions to occur in aqueous solutions such as in aqueous solutions buffered at pH 6 to 8, and for the resulting conjugate to remain water soluble for more effective bio-distribution. Typically, the polyfunctional molecule covalently bonds with an amino or a sulfhydryl functional group. However, polyfunctional molecules reactive with other functional groups, such as carboxylic acids or hydroxyl groups, are contemplated in the present invention.

The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde. The use of glutaraldehyde as a cross-linking agent was disclosed by Poznansky et al., Science 223, 1304-1306 (1984). Homobifunctional molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts. These homobifunctional reagents are available from numerous commercail sources (Pierce, Rockford, Ill.).

The heterobifunctional molecules have at least two different reactive groups. The reactive groups react with different functional groups, e.g., present on the erythropoietin mutein and the molecule. These two different functional groups that react with the reactive group on the heterobifunctional cross-linker are usually an amino group, e.g., the epsilon amino group of lysine; a sulfbydryl group, e.g., the thiol group of cysteine; a carboxylic acid, e.g., the carboxylate on aspartic acid; or a hydroxyl group, e.g., the hydroxyl group on serine. Of course, recombinant tissue protective cytokines of the invention may be lacking a particular amino acid residue that would facilitate cross-linking of native erythropoietin, but one of skill in the art will be aware of the available residue moieties in a mutein of the invention and cross-link accordingly.

Moreover, the various recombinant tissue protective cytokine molecules of the invention may not have suitable reactive groups available for use with certain cross-linking agents; however, one of skill in the art will be amply aware of the choice of cross-linking agents based on the available groups for cross-linking in an erythropoietin of the invention.

When a reactive group of a heterobifunctional molecule forms a covalent bond with an amino group, the covalent bond will usually be an amido or imido bond. The reactive group that forms a covalent bond with an amino group may, for example, be an activated carboxylate group, a halocarbonyl group, or an ester group. The preferred halocarbonyl group is a chlorocarbonyl group. The ester groups are preferably reactive ester groups such as, for example, an N-hydroxy-succinimide ester group.

The other functional group typically is either a thiol group, a group capable of being converted into a thiol group, or a group that forms a covalent bond with a thiol group. The covalent bond will usually be a thioether bond or a disulfide. The reactive group that forms a covalent bond with a thiol group may, for example, be a double bond that reacts with thiol groups or an activated disulfide. A reactive group containing a double bond capable of reacting with a thiol group is the maleimido group, although others, such as acrylonitrile, are also possible. A reactive disulfide group may, for example, be a 2-pyridyldithio group or a 5,5′-dithio-bis-(2-nitrobenzoic acid) group. Some examples of heterobifunctional reagents containing reactive disulfide bonds include N-succinimidyl 3-(2-pyridyl-dithio)propionate (Carlsson, et al., 1978, Biochem J., 173:723-737), sodium S-4-succinimidyloxycarbonyl-alpha-methylbenzylthiosulfate, and 4-succinimidyloxycarbonyl-alpha-methyl-(2-pyridyldithio)toluene. N-succinimidyl 3-(2-pyridyldithio)propionate is preferred. Some examples of heterobifunctional reagents comprising reactive groups having a double bond that reacts with a thiol group include succinimidyl 4-(N-maleimidomethyl)cyclohexane-1-carboxylate and succinimidyl m-maleimidobenzoate.

Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-N-hydroxy-succinimide ester. The sodium sulfonate salt of succinimidyl m-maleimidobenzoate is preferred. Many of the above-mentioned heterobifunctional reagents and their sulfonate salts are available from Pierce Chemical Co., Rockford, Ill. USA.

The need for the above-described conjugated to be reversible or labile may be readily determined by the skilled artisan. A conjugate may be tested in vitro for both the recombinant tissue protective cytokine activity, and for the desirable pharmacological activity. If the conjugate retains both properties, its suitability may then be tested in vivo. If the conjugated molecule requires separation from the recombinant tissue protective cytokine for activity, a labile bond or reversible association with the recombinant tissue protective cytokine will be preferable. The lability characteristics may also be tested using standard in vitro procedures before in vivo testing.

Additional information regarding how to make and use these as well as other polyfunctional reagents may be obtained from the following publications or others available in the art:

1. Carlsson, J. et al., 1978, Biochem. J. 173:723-737.

2. Cumber, J. A. et al., 1985, Methods in Enzymology 112:207-224.

3. Jue, R. et al., 1978, Biochem 17:5399-5405.

4. Sun, T. T. et al., 1974, Biochem. 13:2334-2340.

5. Blattler, W. A. et al., 1985, Biochem. 24:1517-152.

6. Liu, F. T. et al., 1979, Biochem. 18:690-697.

7. Youle, R. J. and Neville, D. M. Jr., 1980, Proc. Natl. Acad. Sci. U.S.A. 77:5483-5486.

8. Lemer, R. A. et al., 1981, Proc. Natl. Acad. Sci. U.S.A. 78:3403-3407.

9. Jung, S. M. and Moroi, M., 1983, Biochem. Biophys. Acta 761:162.

10. Caulfield, M. P. et al., 1984, Biochem. 81:7772-7776.

11. Staros, J. V., 1982, Biochem. 21:3950-3955.

b 12. Yoshitake, S. et al., 1979, Eur. J Biochem. 101:395-399.

13. Yoshitake, S. etal., 1982, J. Biochem. 92:1413-1424.

14. Pilch, P. F. and Czech, M. P., 1979, J. Biol. Chem. 254:3375-3381.

15. Novick, D. et al., 1987, J. Biol. Chem. 262:8483-8487.

16. Lomant, A. J. and Fairbanks, G., 1976, J. Mol. Biol. 104:243-261.

17. Hamada, H. and Tsuruo, T., 1987, Anal. Biochem. 160:483-488.

18. Hashida, S. et al., 1984, J. Applied Biochem. 6:56-63.

Additionally, methods of cross-linking are reviewed by Means and Feeney, 1990, Bioconjugate Chem. 1:2-12.

Barriers which are crossed by the above-described methods and compositions of the present invention include, but are not limited to, the blood-brain barrier, the blood-eye barrier, the blood-testis barrier, the blood-ovary barrier, the blood-heart barrier, the blood-kidney barrier, and the blood-uterus barrier.

Candidate molecules for transport across an endothelial cell barrier include, for example, hormones, such as growth hormone, neurotrophic factors, antibiotics, antivirals, or antiftngals such as those normally excluded from the brain and other barriered organs, peptide radiopharmaceuticals, antisense drugs, antibodies and antivirals against biologically-active agents, pharmaceuticals, and anti-cancer agents. Non-limiting examples of such molecules include hormones such as growth hormone, nerve growth factor (NGF), brain-derived neurotrophic factor (BDNF), ciliary neurotrophic factor (CNTF), basic fibroblast growth factor (bFGF), transforming growth factor β1 (TGFβ1), transforming growth factor β2 (TGFβ2), transforming growth factor β3 (TGFβ3), interleukin 1, interleukin 2, interleukin 3, and interleukin 6, AZT, antibodies against tumor necrosis factor, and immunosuppressive agents such as cyclosporin. Additionally, dyes or markers may be attached to erythropoietin or one of the tissue protective cytokines of the present invention in order to visualize cells, tissues, or organs within the brain and other barriered organs for diagnostic purposes. As an example, a marker used to visualize plaque within the brain could be attached to erythropoietin or a tissue protective cytokine in order to determine the progression of Alzheimers disease within a patient.

The present invention is also directed to a composition comprising a molecule to be transported via transcytosis across a endothelial cell tight junction barrier and a recombinant tissue protective cytokine as described above. The invention is further directed to the use of a conjugate between a molecule and a recombinant tissue protective cytokine as described above for the preparation of a pharmaceutical composition for the delivery of the molecule across a barrier as described above.

The present invention may be better understood by reference to the following non-limiting examples, which are provided as exemplary of the invention. The following examples are presented in order to more fully illustrate the preferred embodiments of the invention. They should in no way be construed, however, as limiting the broad scope of the invention.

### 6. EXAMPLES

### 6.1. Example 1

### Distribution of Erythropoietin Receptor in Human Brain

Normal human brain removed during surgical procedures (e.g., to provide tumor-free margins in tumor resections) were immediately fixed in 5% acrolein in 0.1 M phosphate buffer (pH 7.4) for 3 h. Sections were cut with a vibrating microtome at 40 micrometer thickness. Immunohistochemical staining was performed using free-floating sections and the indirect antibody peroxidase-antiperoxidase method using a 1:500 dilution of erythropoietin receptor antiserum (obtained from Santa Cruz Biotechnology). Endogenous peroxidase activity was quenched by pretreatment of tissue sections with hydrogen peroxide (3% in methanol for 30 min). Tissue controls were also carried out by primary antibody omission and by using the appropriate blocking peptide (from Santa Cruz Biotech.) to confirm that staining was specific for erythropoietin (EPO) receptor.

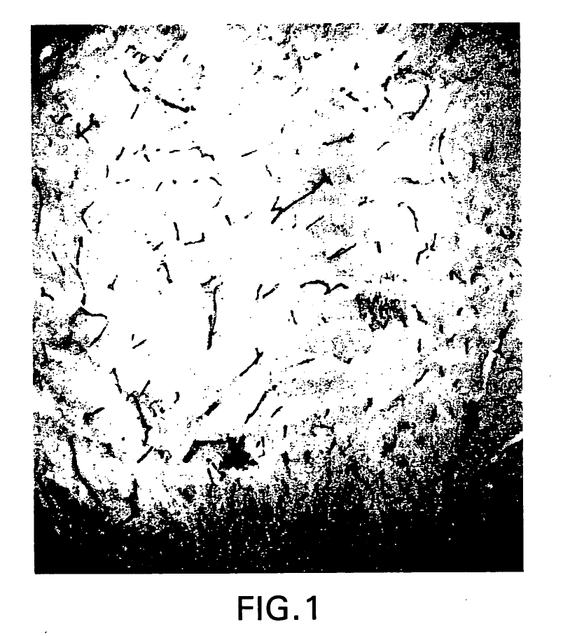

FIG. 1 shows capillaries of the human brain express very high levels of EPO receptor, as determined by immunohistochemistry using specific anti-EPO receptor antibodies. This provides a mechanism whereby EPO is able to penetrate into the brain from the systemic circulation, in spite of the blood brain barrier.

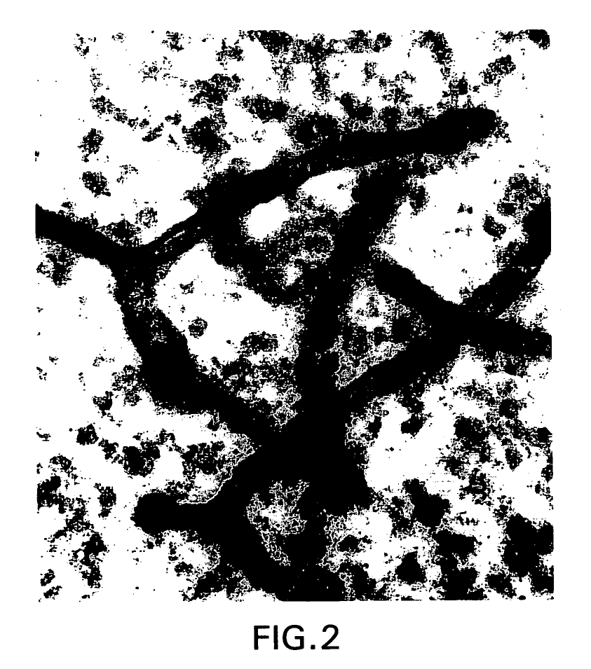

FIG. 2 shows the EPO receptor is densely localized within and around capillaries forming the blood brain barrier in the human brain.

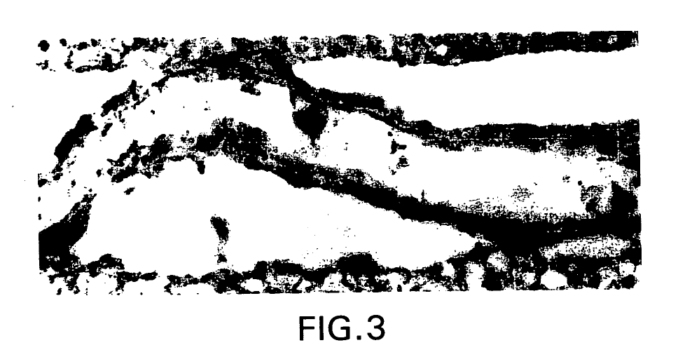

A similar protocol as for FIGS. 1 & 2 was performed for FIG. 3, except that 10 micrometer sections were cut from paraffin, the embedded sections fixed by immersion in 4% paraformaldehyde. FIG. 3 shows that there is a high density of EPO receptor at the luminal and anti-luminal surfaces of human brain capillaries, forming the anatomical basis for transport of EPO from the circulation into the brain.

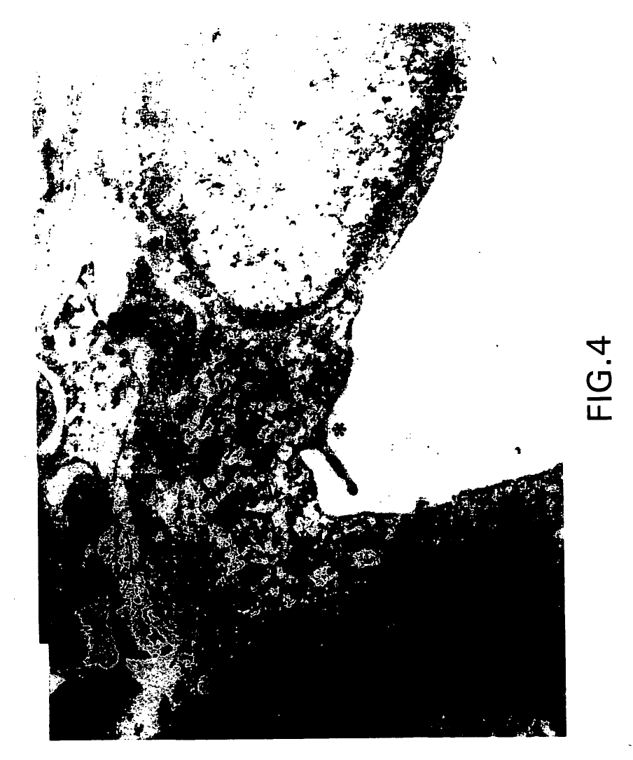

FIG. 4 was obtained following a similar protocol as in FIG. 3 except that the tissue was sectioned on an ultramicrotome for electron microscopy and the secondary antibody was labeled with colloidal gold particles. This figure shows that EPO receptor is found upon the endothelial surface (*), within cytoplasmic vesicles (arrows) and upon glial endfeet (+) in human brain, providing the anatomical basis for transport of EPO from within the circulation into the brain.

### 6.2. Example 2

### Erythropoietin Crosses the Blood-Cerebrospinal Fluid Tight Barrier

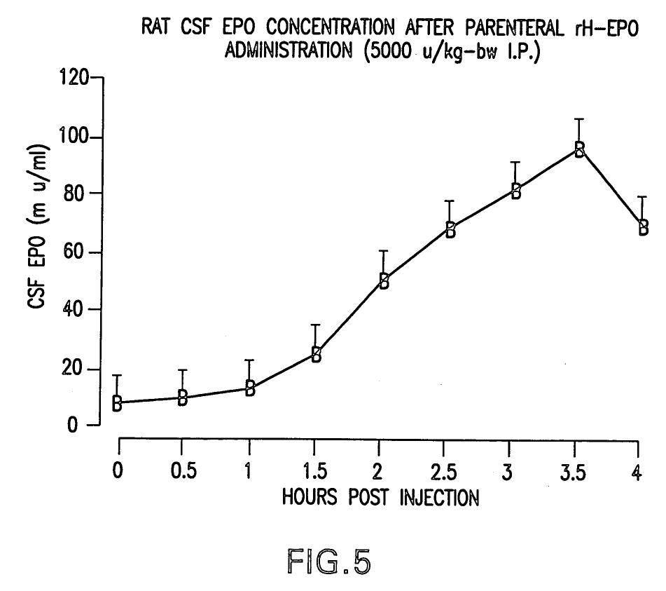

Adult male Sprague-Dawley rats were anesthetized and administered recombinant human erythropoietin intraperitoneally at 5000 U/kg body weight. Cerebrospinal fluid was sampled from the cisterna magna at 30 minute intervals up to 4 hrs and the erythropoietin concentration determined using a sensitive and specific enzyme-linked immunoassay. As illustrated in FIG. 5, the baseline erythropoietin concentration in CSF is 8 mU/ml. After a delay of several hours, the levels of erythropoietin measured in the CSF begin to rise and by 2.5 hours and later are significantly different from the baseline concentration at the p<0.01 level. The peak level of about 100 mU/ml is within the range known to exert protective effects in vitro (0.1 to 100 mU/ml). The time to peak occurs at about 3.5 hrs, which is delayed significantly from the peak serum levels which occur at less than 1 hr. The results of this experiment illustrate that significant levels of erythropoietin can be accomplished across a tight cellular junction by bolus parenteral administration of erythropoietin at appropriate concentrations.

### 6.3. Example 3

### Recombinant Tissue Protective Cytokine

The following human erythropoietin constructs were made using the following procedures. The cDNA for the human erythropoietin was cloned by PCR from human brain cDNA by using primers based on the published human cDNA sequence (accession number NM_{—}000799). The primers were designed to introduce a Xho I site in the 5′ end and a Xba I site in the 3′ end of the cDNA. The primer sequences are:

| | | |
|---|---|---|
| HEPO-5-Xho I | 5′-AGCTCTCGAGGCGCGGAGATGGGGGTGCACGAATG-3′ | (SEQ. ID. 8) |
| HEPO-3-Xba I | 5′-ATGCTCTAGACACACCTGGTCATCTGTCCCCTGTCC-3′. | (SEQ. ID. 9) |

The PCR product was cloned between the Xho I and Xba I sites in pCiNeo mammalian expression vector (Promega). The clones were sequenced and the sequence was verified to match the sequence in NM_{—}000799 with the exception of a single base. Base 418 in the coding sequence (starting the numbering from the ATG) was C instead of G, changing amino acid 140 in the full length EPO sequence starting from the first methionine from Arg to Gly. This is however, normal sequence variation from the original sequence and present in most forms of erythropoietin.

The coding sequence from the erythropoietin cDNA is below:

| | |
|---|---|
| ATGGGGGTGCACGAATGTCCTGCCTGGCTGTGGCTTCTCCTGTCCCTGCT | (SEQ ID NO: 7) |
| GTCGCTCCCTCTGGGCCTCCCAGTCCTGGGCGCCCCACCACGCCTCATCTGTGA | |
| CAGCCGAGTCCTGGAGAGGTACCTCTTGGAGGCCAAGGAGGCCGAGAATATCA | |
| CGACGGGCTGTGCTGAACACTGCAGCTTGAATGAGAATATCACTGTCCCAGACA | |
| CCAAAGTTAATTTCTATGCCTGGAAGAGGATGGAGGTCGGGCAGCAGGCCGTA | |
| GAAGTCTGGCAGGGCCTGGCCCTGCTGTCGGAAGCTGTCCTGCGGGGCCAGGC | |
| CCTGTTGGTCAACTCTTCCCAGCCGTGGGAGCCCCTGCACTGCATGTGGATAAA | |
| GCCGTCAGTGGCCTTCGCAGCCTCACCACTCTGCTTCGGGCTCTGGGAGCCCAG | |
| AAGGAAGCCATCTCCCCTCCAGATGCGGCCTCAGCTGCTCCACTCCGAACAATC | |
| ACTGCTGACACTTTCGCAAACTCTTCCGAGTCTACTCCAATTTCCTCCGGGGAA | |
| AGCTGAAGCTGTACACAGGGGAGGCCTGCAGGACAGGGGACAGATGA. | |

This cDNA codes for the full length amino acid sequence of erythropoietin, which is below

| | |
|---|---|
| MGVHECPAWLWLLLSLLSLPLGLPVLGAPPRLICDSRVLERYLLEAKEAENI | (SEQ ID NO: 10) |
| TTGCAEHCSLNENITVPDTKVNFYAWKRMEVGQQAVEVWQGLALLSEAVLRGQA | |
| LLVNSSQPWEPLQLHVDKAVSGLRSLTTLLRALGAQKEAISPPDAASAAPLRTITAD | |
| TFRKLFRVYSNFLRGKLKLYTGEACRTGDR. | |

The first 27 amino acid residues of SEQ ID NO:10 comprise a leader sequence.

A 6×His tag was introduced to the C-terminal end of the human EPO protein by designing a new oligonucleotide so that the 6 histidines would be joined to the Asp 192 in the full length sequence using the following oligonucleotide:

3′-6×his- hEPO 5′-GGTCTAGATCAATGGTGATGGTGATGATGGTCCCCTGTCCTGCAGGCC-3′ (SEQ ID NO:134)

The EPO cDNA was amplified by PCR using the HEPO-5-Xho I oligo and 6xHis-Tag oligo and cloned between the Xho I and Xba I sites in the pCiNeo mammalian expression vector. The insert was again sequenced and the sequence verified.

The mutations described above in section 5.2 to the human EPO cDNA sequence, with a C-terminal 6×His tag, were introduced by oligo directed mutagenesis using the oligonucleotides described in this section. Mutant clones were sequenced to confirm the mutations.

Numerous methods for purification of the recombinant tissue protective cytokines of the invention may be used, including, but not limited to, the following protocol which was used in conjunction with the histidine tagged recombinantly expressed tissue protective cytokines of the invention. The recombinant cell (CHO-K1) supernatant (for example, resin from (Ni-CAM HC RESIN: High Capacity Nickel Chelate Affinity Matrix, Sigma, Cat no. N 3158)) was thoroughly resuspend with gentle inversion. Then, 100 μl of the resin suspension (equivalent to 50 μl of packed resin) was added to a microcentrifuge tube (1.7 ml size). The mixture was centrifuged at 8,000 rpm, at 4° C. for 5 minutes to pellet the resin and then discard the supernatant. The microcentrifuge was Megafuge 1.0 R (Heraeus Instruments). The mixture was washed twice with 1 ml of deionized water (0.2 μm filtered) to remove the ethanol. The resin was resuspended in 500 μl of equilibration buffer (50 mM sodium phosphate, pH 8.0, 0.3 M NaCl, 10 mM imidazole), and then transferred the mixture to a 50-ml conical tube. The microcentrifuge tube was rinsed with 500 μl of equilibration buffer, and then added this amount to the mixture in the 50-ml conical tube. The mixture was centrifuged at 3,000 rpm, at 4° C. for 5 minutes to pellet the resin. The supernatant was removed and discarded. The samples (CHO-KI supernatant) were centrifuged at 3,800 rpm, at 4° C. for 5 minutes prior to binding. The cell supernatant was added to the resin. Sample addition buffer (50 mM sodium phosphate, pH 8.0, 3 M NaCl, 100 mM imidazole) was added to 1×, and gently mixed in a rotating platform for 1 hour at 4° C. An example of such a platform used is Nutator (rotating platform) (Clay Adams Brand). The mixture was centrifuged at 3,000 rpm, at 4° C. for 5 minutes. The supernatant was removed and saved for SDS-PAGE analysis and ELISA (unbound). The resin was resuspended in 500 μl of wash buffer, and then the mixture was transferred to a microcentrifuge tube. The 50-ml conical tube was rinsed with 500 μl of equilibration buffer, and then this amount was added to the mixture in the microcentrifuge tube. The resin suspension was then mixed in a rotating platform for 10 minutes at 4° C. The suspension was centrifuged at 8,000 rpm, at 4° C. for 5 minutes (the first wash may be saved for ELISA). The resin was resuspended in 1 ml of wash buffer and the resin suspension was again then mixed in a rotating platform for 10 minutes at 4° C., to wash the resin one more time. The wash was disgarded. Then, 75 μl of elution buffer (50 mM sodium phosphate, pH 8.0, 0.3 M NaCl, 500 mM imidazole) was added. The resin was mixed in a rotating platform for 10 minutes at 4° C. The mixture was centrifuged at 8,000 rpm, at 4° C. for 5 minutes. The supernatant was removed and saved. The histidine tagged protein was in this fraction. To elute more protein, 75 μl of elution buffer (50 mM sodium phosphate, pH 8.0, 0.3 M NaCl, 500 mM imidazole) were again added. The resin was again mixed in a rotating platform for 10 minutes at 4° C. The mixture was again centrifuged at 8,000 rpm, at 4° C. for 5 minutes. The eluted fractions were saved as a single pool or separate fractions.

Alternatively, the following procedure was used to isolate purified histidine-tagged cytokines. Conditioned media was collected and filtered through a 0.45 μm filter. A 50 ml aliquot was then applied to a 5 ml HiTrap chelating (Amersham biosciences) equilibrated with 20 mM sodium phosphate pH 7.4 and activated with 2.5 ml 100 mM NiSO₄.The column was washed with 20 mM sodium phosphate pH 7.4 and eluted with a gradient from 0 M to 0.5 M Imidazole in 20 mM sodium phosphate pH 7.4 over 25 column volumes. The flow was 5 ml/min and fraction size 5 ml.

Fractions were analyzed for the presence of recombinant tissue protective cytokine by SDS-PAGE and EPO ELISA. Positive fractions were pooled and dialyzed against 10 mM Tris pH 7.0. The pool was applied to a 1 ml HiTrap Q HP (Amersham biosciences) equilibrated with 10 mM Tris pH 7.0. After washing with equilibration buffer the sample was eluted with a gradient of NaCl to 0.5 M over 10 column volumes at a flow of 1 ml/min. Fractions of 1 ml were collected and analyzed by SDS-PAGE, EPO ELISA and western blotting using antibodies against hexa-his tag (Anti-HiS₆, ROCHE). Fractions containing the recombinant tissue protective cytokine were pooled and concentrated using a centricon with a cut off size of 10 kDa (Amicon) to a final volume of 1-2 ml.

The final pool of recombinant tissue protective cytokine was analyzed by SDS-PAGE (NuPage 4-12%; Invitrogen) and visualized using NOVEX Colloidal Blue (Invitrogen) by the protocol recommended by the manufacturer. The purity of the recombinant tissue protective cytokine was judged from the resulting gel. Only one band corresponding to the glycosylated recombinant tissue protective cytokine was present in each lane of the gel indicating a high purity of the isolated cytokine.

All the plasmids were transfected to either CHO-1 cells or COS-7 cells by using lipofectamine. Forty-eight to 72 hours post transfection media from the cells was collected. This media was tested for EPO by ELISA assay and used either directly or after purification in either the hematopoietic or neuronal assays.

Mutations K45D, S100E, and A30N/H32T to the human EPO cDNA sequence were introduced by oligo directed mutagenesis using following oligonucleotides:

| | |
|---|---|
| HEPO-S100E-upper 5′- | (SEQ ID NO: 11) |
| CATGTGGATAAAGCCGTCGAGGGCCTTCGCAGCCTCACCACTCTG-3′ | |
| HEPO-S100E-lower 5′- | (SEQ ID NO: 12) |
| CAGAGTGGTGAGGCTGCGAAGGCCCTCGACGGCTTTATCCACATG-3′ | |
| HEPO-K45D-upper 5′- | (SEQ ID NO: 13) |
| GAGAATATCACTGTCCCAGACACCGACGTTAATTTCTATGCCTGG-3′ | |
| HEPO-K45D-lower 5′- | (SEQ ID NO: 14) |
| CCAGGCATAGAAATTAACGTCGGTGTCTGGGACAGTGATATTCTC-3′ | |
| hEPO-A30N/H32T-upper 5′- | (SEQ ID NO: 132) |
| GAATATCACGACGGGCTGTAATGAAACCTGCAGCTTGAATGAG-3′ | |
| hEPO-A30N/H32T-lower 5′- | (SEQ ID NO: 133) |
| CTCATTCAAGCTGCAGGTTTCATTACAGCCCGTCGTGATATTC-3′ | |

Oligonucleotide sequences used in oligo directed mutagenesis for the other erythropoietin muteins and recombinant tissue protective cytokines of the invention include:

| | |
|---|---|
| For R150E mutein: | |
| R150E-F GTCTACTCCAATTTCCTCGAGGGAAAGCTGAAGCTG, | (SEQ ID NO: 120) |
| R150E-R GCTTCAGCTTTCCCTCGAGGAAATTGGAGTAGAC | (SEQ ID NO: 121) |

| | |
|---|---|
| For R103E mutein: | |
| R103E-F CCGTCAGTGGCCTTGAGAGCCTCACCACTCTG, | (SEQ ID NO: 122) |
| R103E-R CAGAGTGGTGAGGCTCTCAAGGCCACTGACGG | (SEQ ID NO: 123) |
| For R103E/L108S(103) combination mutein: | |
| R103E-F CCGTCAGTGGCCTTGAGAGCCTCACCACTCTG | (SEQ ID NO: 124) |
| R103E-R CAGAGTGGTGAGGCTCTCAAGGCCACTGACGG | (SEQ ID NO: 125) |
| L108S(103)F CGCAGCCTCACCACTTCGCTTCGGGCTCTGG, | (SEQ ID NO: 126) |
| L108S(103)R CCAGAGCCCGAAGCGAAGTGGTGAGGCTGCG | (SEQ ID NO: 127) |
| For 44-49 deletion | |
| d44-49F GAATATCACTGTCCCAGACGGTGGTGCCTGGAAGAGGATG, | (SEQ ID NO: 128) |
| d44-49R CATCCTCTTCCAGGCACCACCGTCTGGGACAGTGATATTC | (SEQ ID NO: 129) |
| For K20A mutein: | |
| K20A-F TACCTCTTGGAGGCCGCGGAGGCCGAGAATATC, | (SEQ ID NO: 130) |
| K20A-R GATATTCTCGGCCTCCGCGGCCTCCAAGAGGTA | (SEQ ID NO: 131) |
| For K140A mutein: | |
| K140A-F GCTGACACTTTCCGCGCACTCTTCCGAGTCTACTC, | (SEQ ID NO: 132) |
| K140A-R GAGTAGACTCGGAAGAGTGCGCGGAAAGTGTCAGC | (SEQ ID NO: 133) |
| For K152A mutein: | |
| K152A-F ATTTCCTCCGGGGAGCGCTGAAGCTGTACACAG, | (SEQ ID NO: 134) |
| K152A-R CTGTGTACAGCTTCAGCGCTCCCCGGAGGAAAT | (SEQ ID NO: 135) |
| For K154A mutein: | |
| K154A-F CTCCGGGGAAAGCTGGCGCTGTACACAGGGGA, | (SEQ ID NO: 136) |
| K154A-R TCCCCTGTGTACAGCGCCAGCTTTCCCCGGAG | (SEQ ID NO: 137) |
| For K45A mutein: | |
| K45A-F ACTGTCCCAGACACCGCAGTTAATTTCTATGCCTG, | (SEQ ID NO: 138) |
| K45A-R CAGGCATAGAAATTAACTGCGGTGTCTGGGACAGT | (SEQ ID NO: 139) |
| For K52A mutein: | |
| K52A-F AGTTAATTTCTATGCCTGGGCGAGGATGGAGGTCG, | (SEQ ID NO: 140) |
| K52A-R CGACCTCCATCCTCGCCCAGGCATAGAAATTAACT | (SEQ ID NO: 141) |
| For K97A mutein: | |
| K97A-F TGCAGCTGCATGTGGATGCAGCCGTCAGTGGCC, | (SEQ ID NO: 142) |
| K97A-R GGCCACTGACGGCTGCATCCACATGCAGCTGCA | (SEQ ID NO: 143) |
| For K116A mutein: | |
| K116A-F CTCTGGGAGCCCAGGCGGAAGCCATCTCCCCT, | (SEQ ID NO: 144) |
| K116A-R AGGGGAGATGGCTTCCGCCTGGGCTCCCAGAG | (SEQ ID NO: 145) |
| For K140A/K52A combination mutein: | |
| K140A-F GCTGACACTTTCCGCGCACTCTTCCGAGTCTACTC, | (SEQ ID NO: 146) |
| K140A-R GAGTAGACTCGGAAGAGTGCGCGGAAAGTGTCAGC | (SEQ ID NO: 147) |
| K52A-F AGTTAATTTCTATGCCTGGGCGAGGATGGAGGTCG, | (SEQ ID NO: 148) |
| K52A-R CGACCTCCATCCTCGCCCAGGCATAGAAATTAACT | (SEQ ID NO: 149) |
| For K140A/K52A/K45A combination mutein: | |
| K140A-F GCTGACACTTTCCGCGCACTCTTCCGAGTCTACTC, | (SEQ ID NO: 150) |
| K140A-R GAGTAGACTCGGAAGAGTGCGCGGAAAGTGTCAGC | (SEQ ID NO: 151) |
| K52A-F AGTTAATTTCTATGCCTGGGCGAGGATGGAGGTCG, | (SEQ ID NO: 152) |
| K52A-R CGACCTCCATCCTCGCCCAGGCATAGAAATTAACT | (SEQ ID NO: 153) |
| K45A-F ACTGTCCCAGACACCGCAGTTAATTTCTATGCCTG, | (SEQ ID NO: 154) |
| K45A-R CAGGCATAGAAATTAACTGCGGTGTCTGGGACAGT | (SEQ ID NO: 155) |
| For K97A/K152A combination mutein: | |
| K97A-F TGCAGCTGCATGTGGATGCAGCCGTCAGTGGCC, | (SEQ ID NO: 156) |
| K97A-R GGCCACTGACGGCTGCATCCACATGCAGCTGCA | (SEQ ID NO: 157) |
| K152A-F ATTTCCTCCGGGGAGCGCTGAAGCTGTACACAG, | (SEQ ID NO: 158) |
| K152A-R CTGTGTACAGCTTCAGCGCTCCCCGGAGGAAAT | (SEQ ID NO: 159) |
| For K97A/K152A/K45A combination mutein: | |
| K97A-F TGCAGCTGCATGTGGATGCAGCCGTCAGTGGCC, | (SEQ ID NO: 160) |
| K97A-R GGCCACTGACGGCTGCATCCACATGCAGCTGCA | (SEQ ID NO: 161) |
| K152A-F ATTTCCTCCGGGGAGCGCTGAAGCTGTACACAG, | (SEQ ID NO: 162) |
| K152A-R CTGTGTACAGCTTCAGCGCTCCCCGGAGGAAAT | (SEQ ID NO: 163) |
| K45A-F ACTGTCCCAGACACCGCAGTTAATTTCTATGCCTG, | (SEQ ID NO: 164) |
| K45A-R CAGGCATAGAAATTAACTGCGGTGTCTGGGACAGT | (SEQ ID NO: 165) |
| For K97A/K152A/K45A/K52A combination mutein: | |
| K97A-F TGCAGCTGCATGTGGATGCAGCCGTCAGTGGCC, | (SEQ ID NO: 166) |
| K97A-R GGCCACTGACGGCTGCATCCACATGCAGCTGCA | (SEQ ID NO: 167) |
| K152A-F ATTTCCTCCGGGGAGCGCTGAAGCTGTACACAG, | (SEQ ID NO: 168) |
| K152A-R CTGTGTACAGCTTCAGCGCTCCCCGGAGGAAAT | (SEQ ID NO: 169) |
| K45A-F ACTGTCCCAGACACCGCAGTTAATTTCTATGCCTG, | (SEQ ID NO: 170) |
| K45A-R CAGGCATAGAAATTAACTGCGGTGTCTGGGACAGT | (SEQ ID NO: 171) |
| K52A-F AGTTAATTTCTATGCCTGGGCGAGGATGGAGGTCG, | (SEQ ID NO: 172) |
| K52A-R CGACCTCCATCCTCGCCCAGGCATAGAAATTAACT | (SEQ ID NO: 173) |
| For K97A/K152A/K45A/K52A/K140A combination mutein: | |
| K97A-F TGCAGCTGCATGTGGATGCAGCCGTCAGTGGCC, | (SEQ ID NO: 174) |
| K97A-R GGCCACTGACGGCTGCATCCACATGCAGCTGCA | (SEQ ID NO: 175) |
| K152A-F ATTTCCTCCGGGGAGCGCTGAAGCTGTACACAG, | (SEQ ID NO: 176) |
| K152A-R CTGTGTACAGCTTCAGCGCTCCCCGGAGGAAAT | (SEQ ID NO: 177) |
| K45A-F ACTGTCCCAGACACCGCAGTTAATTTCTATGCCTG, | (SEQ ID NO: 178) |
| K45A-R CAGGCATAGAAATTAACTGCGGTGTCTGGGACAGT | (SEQ ID NO: 179) |
| K52A-F AGTTAATTTCTATGCCTGGGCGAGGATGGAGGTCG, | (SEQ ID NO: 180) |
| K52A-R CGACCTCCATCCTCGCCCAGGCATAGAAATTAACT | (SEQ ID NO: 181) |
| K140A-F GCTGACACTTTCCGCGCACTCTTCCGAGTCTACTC, | (SEQ ID NO: 182) |
| K140A-R GAGTAGACTCGGAAGAGTGCGCGGAAAGTGTCAGC | (SEQ ID NO: 183) |
| For K97A/K152A/K45A/K52A/K140A/K154A combination mutein: | |
| K97A-F TGCAGCTGCATGTGGATGCAGCCGTCAGTGGCC, | (SEQ ID NO: 184) |
| K97A-R GGCCACTGACGGCTGCATCCACATGCAGCTGCA | (SEQ ID NO: 185) |
| K152A-F ATTTCCTCCGGGGAGCGCTGAAGCTGTACACAG, | (SEQ ID NO: 186) |
| K152A-R CTGTGTACAGCTTCAGCGCTCCCCGGAGGAAAT | (SEQ ID NO: 187) |
| K45A-F ACTGTCCCAGACACCGCAGTTAATTTCTATGCCTG, | (SEQ ID NO: 188) |
| K45A-R CAGGCATAGAAATTAACTGCGGTGTCTGGGACAGT | (SEQ ID NO: 189) |
| K52A-F AGTTAATTTCTATGCCTGGGCGAGGATGGAGGTCG, | (SEQ ID NO: 190) |
| 52A-R CGACCTCCATCCTCGCCCAGGCATAGAAATTAACT | (SEQ ID NO: 191) |
| K140A-F GCTGACACTTTCCGCGCACTCTTCCGAGTCTACTC, | (SEQ ID NO: 192) |
| K140A-R GAGTAGACTCGGAAGAGTGCGCGGAAAGTGTCAGC | (SEQ ID NO: 193) |
| K154A(152)F CTCCGGGGAGCGCTGGCGCTGTACACAGGGGA, | (SEQ ID NO: 194) |
| 154(152)R TCCCCTGTGTACAGCGCCAGCGCTCCCCGGAG | (SEQ ID NO: 195) |
| For N24K/N38K/N83K combination mutein: | |
| N24K-F CAAGGAGGCCGAGAAAATCACGACGGGCTGT, | (SEQ ID NO: 196) |
| N24K-R ACAGCCCGTCGTGATTTTCTCGGCCTCCTTG | (SEQ ID NO: 197) |
| N38K-F ACTGCAGCTTGAATGAGAAAATCACTGTCCCAGACAC, | (SEQ ID NO: 198) |
| N38K-R GTGTCTGGGACAGTGATTTTCTCATTCAAGCTGCAGT | (SEQ ID NO: 199) |
| N83K-F AGGCCCTGTTGGTCAAATCTTCCCAGCCGTG, | (SEQ ID NO: 200) |
| N83K-R CACGGCTGGGAAGATTTGACCAACAGGGCCT | (SEQ ID NO: 201) |
| For K152W mutein: | |
| K152W-F ATTTCCTCCGGGGATGGCTGAAGCTGTACACAG, | (SEQ ID NO: 202) |
| K152W-R CTGTGTACAGCTTCAGCCATCCCCGGAGGAAAT | (SEQ ID NO: 203) |
| For R14A/Y15A combination mutein: | |
| RY14AA-F AGCCGAGTCCTGGAGGCGGCCCTCTTGGAGGCCAA, | (SEQ ID NO: 204) |
| RY14AA-R TTGGCCTCCAAGAGGGCCGCCTCCAGGACTCGGCT | (SEQ ID NO: 205) |
| Y15A-F AGCCGAGTCCTGGAGAGGGCCCTCTTGGAGGCCAA | (SEQ ID NO: 206) |
| Y15A-R TTGGCCTCCAAGAGGGCCCTCTCCAGGACTCGGCT | (SEQ ID NO: 207) |

The following are examples of constructs that were made: human EPO(hEPO)-6×HisTag-pCiNeo sequence (SEQ ID NO:208); hEPO6×HisTag-A30N/H32T-pCiNeo (SEQ ID NO:209); hEPO-6×HisTag-K45D-pCiNeo sequence (SEQ ID NO:210); hEPO-6×HisTag-S100E-pCiNeo sequence (SEQ ID NO:211); and hEPO-6×HisTag-K45D/S100E-pCiNeo sequence (SEQ ID NO:212). The pCI-neo mammalian expression vector carries the human cytomegalovirus (CMV) immediate-early enhancer/promoter region to promote constitutive expression of cloned DNA inserts in mammalian cells.

These oligonucleotides were annealed to the original human erythropoietin cDNA clone in pCiNeo to introduce the mutations. Mutant clones were sequenced to confirm the mutations. All the plasmids were transfected to either CHO-1 cells or COS-7 cells by using lipofectamine. At 48 to 72 hours post-transfection media from the cells was collected. This media was tested for erythropoietin by ELISA assay and used either directly or after purification in either the hematopoietic or neuronal assays.

Subsequently, both the K45D and S100E recombinant tissue protective cytokines were tested within a neuronal assay. Specifically, an in vitro neuroprotection assay using SK-N-SH neuroblastoma cells was used. SK-N-SH cells were plated at a density of 40,000 cells/well in 24 well plates for 24 hours. Then recombinant tissue protective cytokines were added at a concentration of 3 nM for an additional 24 hours (Erythropoietin=commercial preparation; EPO=erythropoietin and recombinant tissue protective cytokines expressed in CHO cells; pure vector=cell supemantant from CHO cells transfected with vector without Epo construct). After this preincubation, cells were exposed to rotenone (5 μM) for 4 hours, washed, and left to recover for 24 hours. The indicated EPO variants were present during all these steps. Cell viability was quantitated at the end of the experiment by incubation of cells with the tetrazolium dye WST-1 (according to manufacturer's instructions: Roche # 1644807) for 2 hours, and the viability was indicated as absorbance reading.

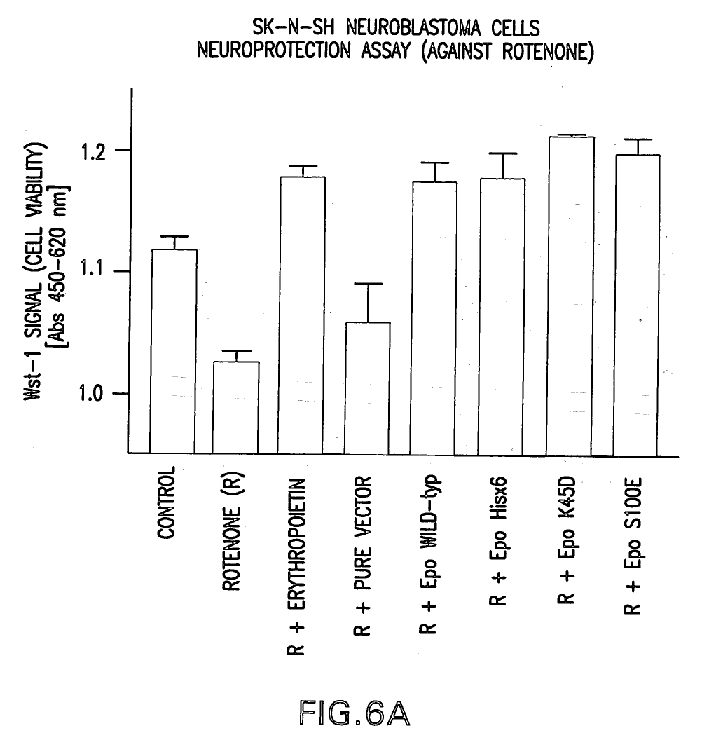

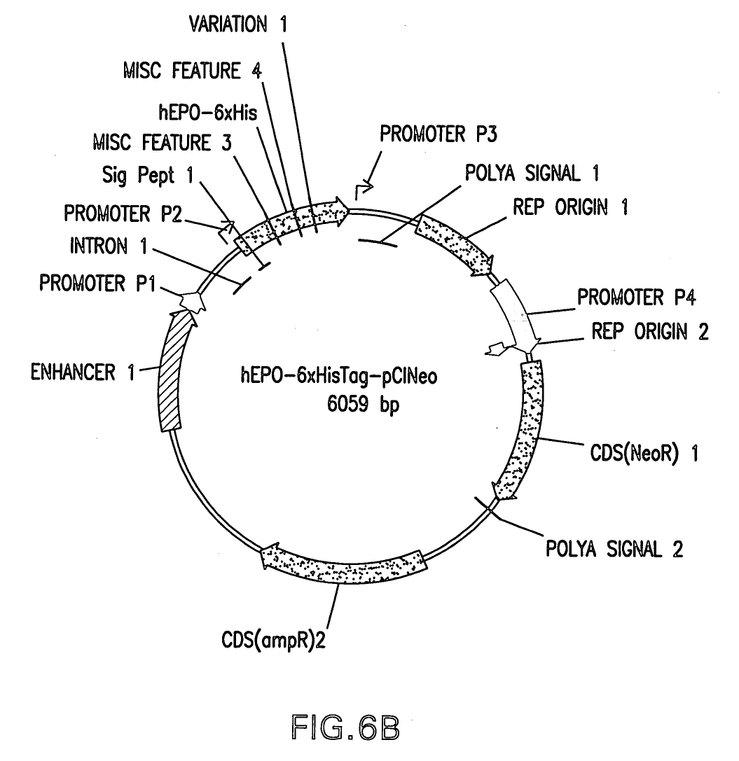

FIGS. 6A and 6B indicates the results of the SK-N-SH neuroblastoma cell neuroprotection assay (against rotenone) for erythropoietin as well as the recombinant tissue protective cytokines with the K45D and S 100E recombinant tissue protective cytokines. The y-axis on the graph indicates the absorbance readings, and the data are means ± range of duplicate determinations. The graph within FIG. 6A clearly indicates that the viability of the cells within the K45D and S100E samples maintained their viability demonstrating their cellular protective effect. FIG. 6B shows the plasmid map of hEPO-6×HisTag-PCiNeo.

### 6.4. Example 4

### Tissue Protective Cytokines

Recombinant tissue protective cytokines desirable for the uses described herein may be further modified by desialation, guanidination, carbamylation, amidination, trinitrophenylation, acetylation, succinylation, nitration, or modification of arginine residues or carboxyl groups, among other procedures. Alternatively, these modifications may be made to native erythropoietin or a derivative of erythropoietin, including, but not limited to, desialylated, guanidinated, carbamylated, amidinated, trinitrophenylated, acetylated, succinylated, or nitrated erythropoietin, prior to its mutation into a recombinant tissue protective cytokines. Some examples of further modifications to recombinant tissue protective cytokines are described below. One of ordinary skill in the art would understand that the procedures listed below may also be used to chemically modify native erythropoietin or its derivatives prior to the introduction of mutations to generate a recombinant tissue protective cytokine.

6.4.1. Desialylating Recombinant Tissue Protective Cytokines

A recombinant tissue protective cytokines may be desialylated by the following exemplary procedure. Sialidase (isolated from Streptococcus sp 6646K.) is obtained from SEIKAGAKU AMERICA (Code No. 120050). The recombinant tissue protective cytokine is subjected to desialylation by sialidase (0.025 U/mg EPO) at 37° C. for 3 h. Desalt and concentrate the reaction mixture by Ultrafree Centrifugal Filter Unit. Apply sample to an ion exchange column in AKTAprime™ system. Elute protein with the selected buffers. Perform IEF gel analysis of the eluted fractions containing a significant amount of protein. Pool the fractions containing only the top two bands (migrating at pI ˜8.5 and ˜7.9 on IEF gel). Determine the protein content and add 1/9 volumes of 10× salt solution (1 M NaCl, 0.2 M sodium citrate, 3 mM citric acid). Determine the sialic acid content. No significant sialic content is detected.

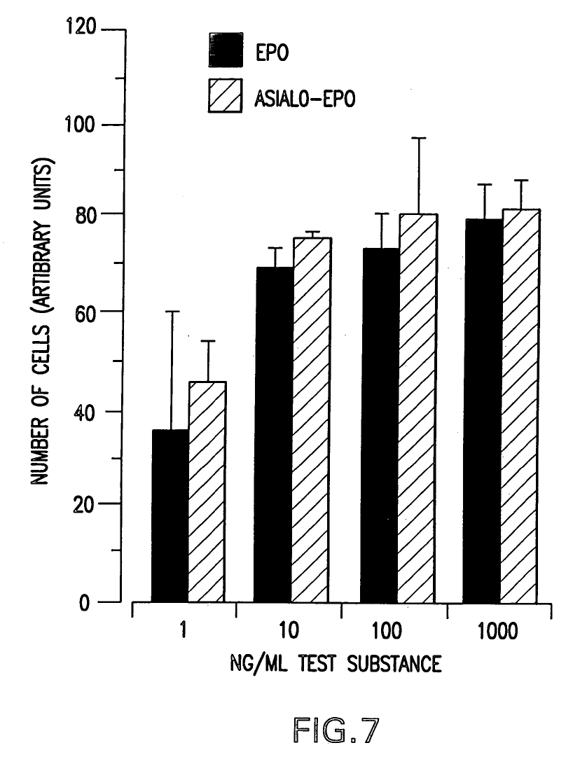

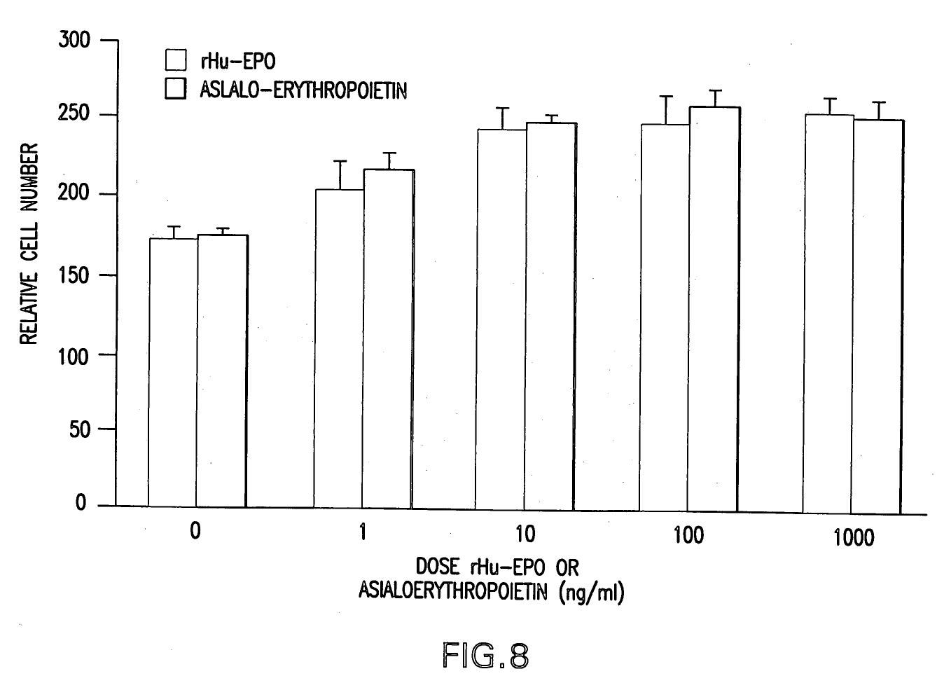

Asialoerythropoietin was as effective as native erythropoietin for neural cells in vitro as shown in FIGS. 7-8. In-vitro testing was carried out using neural-like embryonal carcinoma cells (P19) that undergo apoptosis upon the withdrawal of serum. Twenty-four hours before the removal of serum, 1-1000 ng/ml of erythropoietin or a modified erythropoietin was added to the cultures. The following day the medium was removed, the cells washed with fresh, non-serum containing medium, and medium containing the test substance (no serum) added back to the cultures for an additional 48 hours. To determine the number of viable cells, a tetrazolium reduction assay was performed (CellTiter 96; Promega, Inc.). As FIGS. 7-8 illustrate, asialoerythropoietin appears to be of equal potency to erythropoietin itself in preventing cell death.

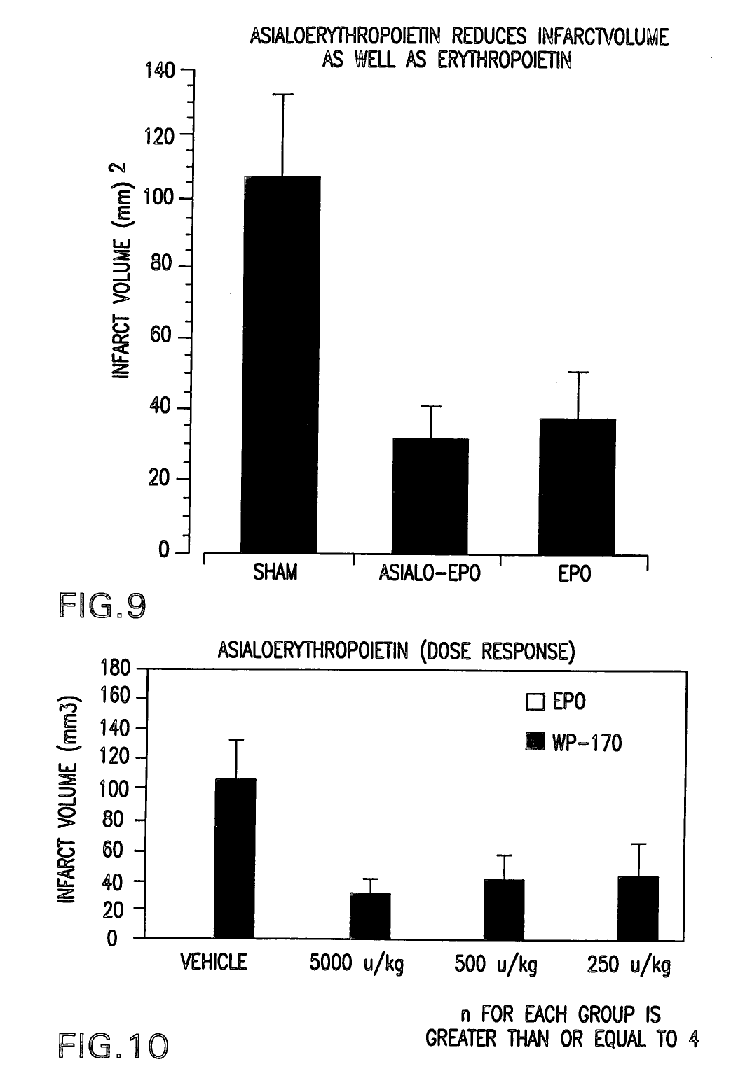

Retention of neuroprotective activity in vivo was confirmed using a rat focal ischemia model in which a reversible lesion in the territory of the middle cerebral artery is performed as described previously (Brines et al., 2000, Proc. Nat. Acad. Sci. U.S.A. 97:10526-31). Adult male Sprague-Dawley rats were administered asialoerythropoietin or erythropoietin (5000 U/kgBW intraperitoneally) or vehicle at the onset of the arterial occlusion. Twenty-four hours later, the animals were sacrificed and their brains removed for study. Serial sections were cut and stained with tetrazolium salts to identify living regions of the brain. As shown in FIG. 9, asialoerythropoietin was as effective as native erythropoietin in providing neuroprotection, i.e. reducing infarctvolume, from 1 hour of ischemia. FIG. 10 shows the results of another focal ischemia model in which a comparative dose response was performed with erythropoietin and asialoerythropoietin. At the lowest dose of 4 μg/kg, asialoerythropoietin afforded protection whereas unmodified erythropoietin did not. The number of rats in each group, n, was greater than or equal to 4.

Similar results would be expected from asialo recombinant tissue protective cytokines of the present invention.

6.4.2. Carbamylating Recombinant Tissue Protective Cytokine

The recombinant tissue protective cytokine may be used to prepare the respective carbamylated molecules, in accordance with the following procedure, as described in Jin Zeng (1991). Lysine modification of metallothionein by carbamylation and guanidination. Methods in Enzymology 205:433-437. Recrystallize potassium cyanate. Prepare 1 M Borate buffer (pH 8.8). Mix a recombinant tissue protective cytokine solution with equal volume of borate buffer. Add potassium cyanate directly to the reaction tube to a final concentration of 0.5 M. Mix well and incubate at 37° C. for 6-16 h. Dialyze thoroughly. Remove the product from the dialysis tubing and collect into a fresh tube. Measure the volume and add 1/9 volume of 10× salt solution (1 M NaCl, 0.2 M sodium citrate, 3 mM citric acid). Determine the protein content and calculate the product recovery rate. Analyze the products by IEF gel followed by an in vitro test with TF-1 cells.

6.4.3. Succinylating Recombinant Tissue Protective Cytokines

The recombinant tissue protective cytokine may be used to prepare the respective succinylated molecule, in accordance with the following procedure, as described in Alcalde et al. (2001). Succinylation of cyclodextrin glycosyltransferase from Thermoanaerobacter sp. 501 enhances its transferase activity using starch as donor. J. Biotechnology 86: 71-80. Recombinant tissue protective cytokine (100 ug) in 0.5 M NaHCO3 (pH 8.0) was incubated with a 15 molar excess of succinic anhydride at 15° C. for 1 hour. The reaction was stopped by dialysis against distilled water.

Dissolve succinic anhydride in dry acetone at 27 mg/ml. Do the reaction in an eppendorf tube in 10 mM sodium phosphate buffer (pH 8.0). Add recombinant tissue protective cytokine protein and 50-fold molar of succinic anhydride to the tube. Mix well and rotate the tube at 4° C. for 1 h. Stop the reaction by dialysis against 10 mM sodium phosphate buffer, using the Dialysis cassette (Slide-A-Laze 7K, Pierce 66373). Remove the product from the dialysis cassette and collect into a fresh tube. Measure the volume and add 1/9 volume of 10× salt solution (1 M NaCl, 0.2 M sodium citrate, 3 mM citric acid). Determine the protein content and calculate the product recovery rate. Analyze the products by IEF gel followed by an in vitro test with TF-1 cells.

6.4.4. Acetylating Recombinant Tissue Protective Cytokine

The recombinant tissue protective cytokine may be used to prepare the respective acetylated molecule, in accordance with the following procedure, as described in Satake et al (1990). Chemical modification of erythropoietin: an increase in in-vitro activity by guanidination. Biochimica et Biophysica Acta. 1038:125-129.

Perform the reaction in an eppendorf tube in 80 mM sodium phosphate buffer (pH 7.2). Add recombinant tissue protective cytokine and equal molar of acetic anhydride. Mix well and incubate on ice for 1 h. Stop the reaction by dialysis against water, using the Dialysis cassette (Slide-A-Laze 7K, Pierce 66373). Remove the product from the dialysis cassette and collect into a fresh tube. Measure the volume and add 1/9 volume of 10× salt solution (1 M NaCl, 0.2 M sodium citrate, 3 mM citric acid). Determine the protein content and calculate the product recovery rate. Analyze the products by IEF gel followed by an in vitro test with TF-1 cells.

6.4.5. Carboxymethylating Lysine of Recombinant Tissue Protective Cytokine

The recombinant tissue protective cytokine may be used to prepare the respective N□-(carboxymethyl)lysine (CML) modified molecules in which one or more lysyl residues of the recombinant tissue protective cytokine are modified, in accordance with the following procedure, as described in Alkhtar et al (1999) Conformational study of N□-(carboxymethyl)lysine adducts of recombinant a-crystallins. Current Eye Research, 18: 270-276.

Freshly prepare 200 mM of glyoxylic acid and 120 mM of NaBH3CN in sodium phosphate buffer (50 mM, pH 7.5). In an eppendorf tube, add recombinant tissue protective cytokine (in phosphate buffer); calculate the lysine equivalent in the solution (about 8 lysine residues/mol). Add 3-times greater NaBH3CN and 5 or 10-times greater glyoxylic acid to the tube. Vortex each tube and incubate at 37° C. for 5 h. Dialyze the samples against phosphate buffer overnight at 4° C. Measure the volume of each product after dialysis. Determine protein concentration and calculate the product recovery rate. Analyze the products by IEF gel followed by an in vitro test with TF-1 cells.

6.4.6. Iodinating Recombinant Tissue Protective Cytokine

A recombinant tissue protective cytokine may be used to prepare the respective iodinated molecule, in accordance with the following procedure, as described in instruction provided by Pierce Chemical Company (Rockford, Ill.) for IODO-Gen Pre-Coated lodination Tubes (product #28601).

Prepare 0.1 M of NaI and perform iodination in IODO-Gen Pre-Coated lodination Tube (Pierce, 28601), with total reaction volume of 0.1 ml/tube in sodium phosphate buffer (40 mM, pH 7.4). Mix the protein substrate (recombinant tissue protective cytokine) with sodium phosphate buffer and then transfer to an IODO-Gen Pre-Coated lodination Tube. Add NaI to final concentration of 1-2 mM, making the molar ration of Nal/protein as 14-20. Mix well and incubate at room temperature for 15 min with gentle agitation. Stop the reaction by removing the reaction mixture and add to a tube containing 3.9 ml of sodium buffer (i.e., a 40-fold dilution). Concentrate the product by a pre-wet Ultrafree centrifugal filter unit. Measure the volume of concentrate and add 1/9 volume of 10× salt solution (1 M NaCl, 0.2 M sodium citrate, 3 mM citric acid). Determine protein concentration and calculate the product recovery rate. Analyze the products by IEF gel followed by an in vitro test with TF-1 cells.

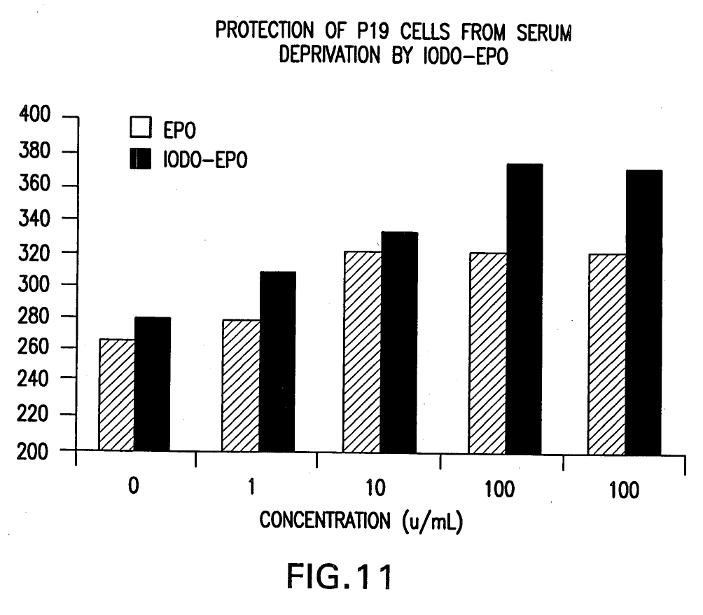

Alternatively the recombinant tissue protective cytokine may be iodinated using the following procedure. One Iodo Bead (Pierce, Rockford, Ill.) was incubated in 100 ul PBS (20 mM sodium phosphate, 0.15 M NaCl, pH7.5) containing 1 mCi free Na125I for 5 minutes. One hundred micrograms of recombinant tissue protective cytokine in 100 ul PBS was then added to the mixture. After a ten minute incubation period at room temperature, the reaction was stopped by removing the 200 ul solution from the reaction vessel (leaving the iodo bead behind). The excess iodine was removed by gel filtration on a Centricon 10 column. As shown in FIG. 11, iodo-erythropoietin produced in this manner is efficacious in protecting P19 cells from serum withdrawal. One of ordinary skill in the art would recognize that similar results would be expected from the iodination of a recombinant tissue protective cytokine of the present invention.

Yet another method for iodinating a recombinant tissue protective cytokine is outlined below. One Hundred micrograms of recombinant tissue protective cytokine in 100 ul PBS was added to 500 uCi Na125I were mixed together in an eppendorf tube. Twenty-five microliters of chloramines T (2 mg/ml) was then added and the mixture was incubated for 1 minute at room temperature. Fifty microliters of Chloramine T stop buffer (2.4 mg/ml sodium metabisulfite, 10 mg/ml tyrosine, 10% glycerol, 0.1% xylene in PBS was then added. The iodotyrosine and iodinated recombinant tissue protective cytokine were then separated by gel filtration on a Centricon 10 column.

6.4.7. Biotinylating Recombinant Tissue Protective Cytokine

A recombinant tissue protective cytokine may be used to prepare the respective biotinylated molecules, in accordance with the following procedure, as described in instruction provided by Pierce Chemical Company (Rockford, Ill.) for EZ-Link NHS-LC-Biotin (product #21336).

Immediately before the reaction, dissolve EZ-Link NHS-LC-Biotin (pierce, 21336) in DMSO at 2 mg/ml. Perform the reaction in a tube (17×100 mm) with total volume of 1 ml containing 50 mM sodium bicarbonate (pH 8.3). Add recombinant tissue protective cytokine and <10% of EZ-Link NHS-LC-Biotin, with molar ratio of Biotin/protein at ˜20. Mix well and incubate on ice for 2 h. Desalt and concentrate the reaction product by Ultrafree centrifugal filter unit. Collect the product into a fresh tube. Measure the volume and add 1/9 volume of 10× salt solution (1 M NaCl, 0.2 M sodium citrate, 3 mM citric acid). Determine the protein content and calculate the product recovery rate. Analyze the products by IEF gel followed by an in vitro test with TF-1 cells.

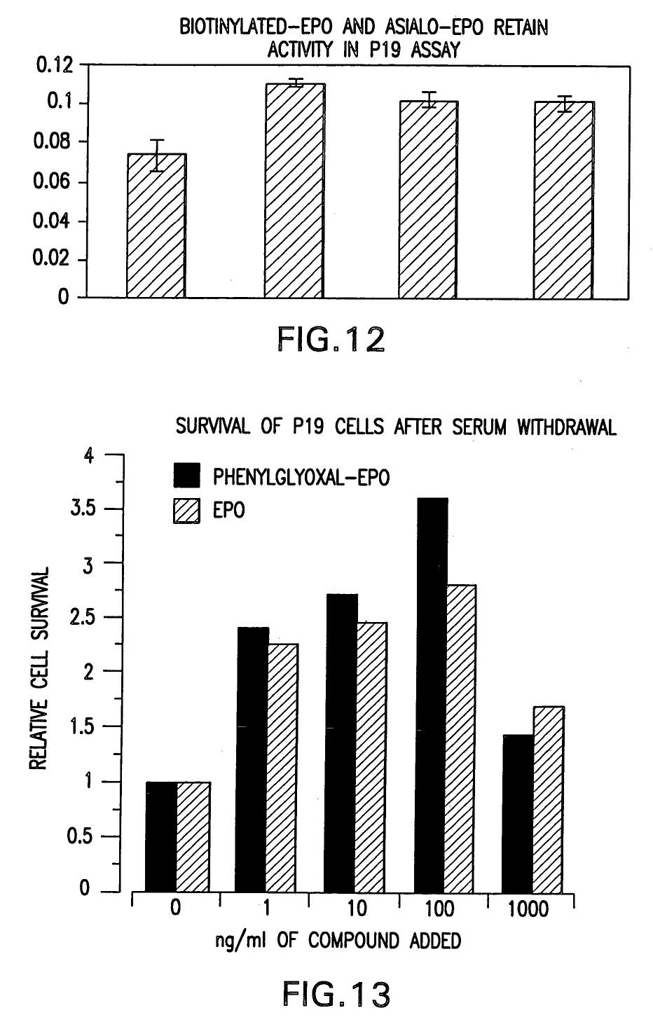

A method for biotinylating the free amino groups of a recombinant tissue protective cytokine is disclosed below. 0.2 mg D-biotinoyl-e-aminocaproic acid-N-hydroxysuccinimide ester (Boehringer Mannheim #1418165) was dissolved in 100 ul DMSO. This solution was combined with 400 ul PBS containing approximately 0.2 mg recombinant tissue protective cytokine in a foil covered tube. After incubation for 4 hours at room temperature, the unreacted biotin was separated by gel filtration on a Centricon 10 column. As shown in FIG. 12, this biotinylated erythropoietin protects p19 cells from serum withdrawal. One of ordinary skill in the art would recognize that similar results would be expected from the biotinylation of a recombinant tissue protective cytokine of the present invention.

Lastly, in “Biotinylated recombinant human erythropoietins: Bioactivity and Utility as a receptor ligand” by Wojchowski et al. Blood, 1989, 74(3):952-8, the authors use three different methods of biotinylating erythropoietin. Biotin is added to (1) the sialic acid moieties; (2) carboxylate groups; and (3) amino groups. The authors use a mouse spleen cell proliferation assay to demonstrate that (1) the addition of biotin to the sialic acid moieties does not inactivate the biological activity of erythropoietin; (2) the addition of biotin to carboxylate groups led to substantial biological inactivation of erythropoietin; (3) the addition of biotin to amino groups resulted in complete biological inactivation of erythropoietin. These methods and modifications are fully embraced herein. FIG. 12 shows the activity of biotinylated erythropoietin and asialoerythropoietin in the serum-starved P19 assay. One of ordinary skill in the art would recognize that similar results would be expected from the iodination of a recombinant tissue protective cytokine of the present invention, see Section 6.15.

### 6.5. Example 5

### Modification of Recombinant Tissues Protective Cytokines by Other Methods

6.5.1. Trinitrophenylation

Recombinant tissue protective cytokine (100 ug) was modified with 2,4,6-trinitrobenzenesulfonate as described in Plapp et al (“Activity of bovine pancreatic deoxyribonuclease A with modified amino groups” 1971, J. Biol. Chem. 246, 939-845).

6.5.2. Arginine modifications

Recombinant tissue protective cytokine was modified with 2,3 butanedione as described in Riordan (“Functional arginyl residues in carboxypeptidase A. Modification with butanedione” Riordan JF, Biochemistry 1973, 12(20): 3915-3923).

In another modification wherein the amino acid residues of erythropoietin are modified, arginine residues were modified by using phenylglyoxal according to the protocol of Takahashi (1977, J Biochem. 81:395-402) carried out for variable lengths of time ranging from 0.5 to 3 hrs at room temperature. The reaction was terminated by dialyzing the reaction mixture against water. Use of such modified forms of erythropoietin is fully embraced herein. The phenylglyoxal-modified erythropoietin was tested using the neural-like P19 cell assay described above. As FIG. 13 illustrates, this chemically-modified erythropoietin fully retains its neuroprotective effects. Similar results form a similarly modified recombinant tissue protective cytokine of the present invention.

A recombinant tissue protective cytokine was modified with cylcohexanone as in Patthy et al (“Identification of functional arginine residues in ribonuclease A and lysozyme” Patthy, L, Smith E L, J. Biol. Chem 1975 250(2): 565-9).

A recombinant tissue protective cytokine was modified with phenylglyoxal as described in Werber et al. (“Proceedings: Carboxypeptidase B: modification of functional arginyl residues” Werber, M M, Sokolovsky M Isr J Med Sci 1975 11(11): 1169-70).

6.5.3. Tyrosine modifications

Recombinant tissue protective cytokine (100 ug) was incubated with tetranitromethane as previously described in Nestler et al “Stimulation of rat ovarian cell steroidogenesis by high density lipoproteins modified with tetranitromethane” Nestler J E, Chacko G K, Strauss J F 3rd. J Biol Chem 1985 Jun 25;260(12):7316-21).

6.5.4. Glutamic Acid (and Aspartic Acid) Modifications

In order to modify carboxyl groups, recombinant tissue protective cytokine (100 ug) was incubated with 0.02 M EDC in 1 M glycinamide at pH 4.5 at room temperature for 60 minutes as described in Carraway et al “Carboxyl group modification in chymotrypsin and chymotrypsinogen.” Carraway K L, Spoerl P, Koshland D E Jr. J Mol Biol 1969 May 28;42(1): 133-7.

6.5.5. Tryptophan Residue Modifications

A recombinant tissue protective cytokine (100 ug) was incubated with 20 uM n-bromosuccinimide in 20 mM potassium phosphate buffer (pH 6.5) at room temperature as described in Ali et al., J Biol Chem. 1995 Mar 3;270(9):4570-4. The number of oxidized tryptophan residues was determined by the method described in Korotchkina (Korotchkina, L G et al Protein Expr Purif. 1995 Feb;6(1):79-90).

6.5.6. Removal of Amino Groups

In order to remove amino groups of recombinant tissue protective cytokines 100 ug was incubated with in PBS (pH 7.4) containing 20 mM ninhydrin (Pierce Chemical, Rockford, Ill.), at 37° C. for two hours as in Kokkini et al (Kokkini, G., et al “Modification of hemoglobin by ninhydrin” Blood, Vol. 556, No 4 1980: 701-705). Reduction of the resulting aldehyde was accomplished by reacting the product with Sodium borohydride or lithium aluminum hydride. Specifically, erythropoietin (100 ug) was incubated with 0.1 M sodium borohydride in PBS for 30 minutes at room temperature. The reduction was terminated by cooling the samples on ice for 10 minutes and dialyzing it against PBS, three times, overnight. (Kokkini, G., Blood, Vol. 556, No 4 1980: 701-705). Reduction using lithium aluminum hydride was accomplished by incubating recombinant tissue protective cytokine (100 ug) with 0.1 M lithium aluminum hydride in PBS for 30 minutes at room temperature. The reduction was terminated by cooling the samples on ice for 10 minutes and dialyzing it against PBS, three times, overnight.

6.5.7. Disulfide Reduction and Stabilization

A recombinant tissue protective cytokine (100 ug) was incubated with 500 mM DTT for 15 minutes at 60° C. 20 mM iodoacetamide in water was then added to the mixture and incubated for 25 minutes, at room temperature in the dark.

6.5.8. Limited Proteolysis

A recombinant tissue protective cytokine can be subjected to a limited chemical proteolysis that targets specific residues. A recombinant tissue protective cytokine can be reacted with 2-(2-nitrophenylsulfenyl)-3-methyl-3′-bromoindolenine which cleaves specifically after tryptophan residues in a 50 times excess in 50% acetic acid for 48 hours in the dark at room temperature in tubes capped under nitrogen pressure. The reaction was terminated by quenching with tryptophan and desalting.

As noted above, a recombinant tissue protective cytokine may be modified, yet multiple modifications as well as additional modifications of the tissue protective cytokine molecule may also be performed without deviating from the spirit of the present invention.

### 6.6. Example 6

### Tissue Protective Cytokines have Neuro Protective Effect

The neuroprotective affects of chemically modified erythropoietin was evaluated using a water intoxication assay in accordance with Manley et al., 2000, Aquaporin-4 deletion in mice reduces brain edema after acute water intoxication and ischemic stroke, Nat Med 2000 Feb;6(2):159-63. Female C3H/HEN mice were used. The mice were given 20% of their body weight as water IP with 400 ng/kg bw DDAVP (desmopressin). The mice were administered erythropoietin (A) or a tissue protective cytokine: asialoerythropoietin (B), carbamylated asialoerythropoietin (C); succinylated asialoerythropoietin (D), acetylated asialoerythropoietin (E); iodinated asialoerythropoietin (F); carboxymethylated asialoerythropoietin (G); carbamylated erythropoietin (H); acetylated erythropoietin (I); iodinated erythropoietin (J) or N^{ε}-carboxy methyl erythropoietin (K). The mice were given a 100 microgram/kg dose of erythropoietin or chemically modified erythropoietin intraperitoneally 24 hrs before administration of the water and again at the time of the water administration. A modified scale from Manley et al. was used to evaluate the mice. The modified scale is as listed below:

| Explores cage/table | |
|---|---|
| Yes | 0 |
| No | 1 |
| Visually tracks objects | |
| Yes | 0 |
| No | 1 |
| Whisker movement | |
| Present | 0 |
| Absent | 1 |
| Leg-tail movements | |
| Normal | 0 |
| Stiff | 1 |
| Paralyzed | 2 |
| Pain withdrawal (toe pinch) | |
| Yes | 0 |
| No | 1 |
| Coordination of movement | |
| Normal | 0 |
| Abnormal | 1 |
| Stops at edge of table | |
| Yes | 0 |
| No | 1 |
| Total score possible: | 8 |

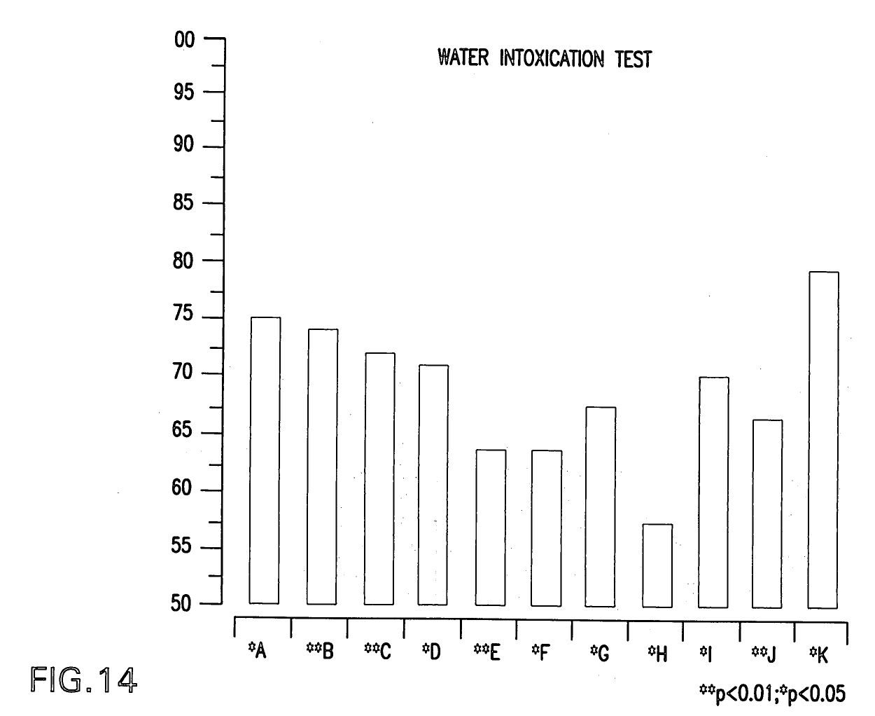

The mice were scored at the following time points: 15, 30, 45, 60, 75, 90, 120, 150, and 180 minutes. FIG. 14 plots the performance of the mice that received erythropoietin or the chemically modified erythropoietin as a percentage of the neuronal deficit experienced by the control mice. FIG. 14 shows that the tissue protective cytokines protect the mice from the neurological trauma induced by the water intoxication. Similar results would be expected from recombinant tissue protective cytokines with similar chemical modifications. Statistical significance was also determined. Those administration regimens with significant differences, p<0.05, in comparison to controls are indicated with *, while those with highly significant differences, p<0.01, are indicated by **.

### 6.7. Example 7

### Maintenance of Function in Heart Prepared for Transplantation

Wistar male rats weighing 300 to 330 g are given erythropoietin (5000 U/kg body weight) or vehicle 24 h prior to removal of the heart for ex vivo studies, done in accordance with the protocol of Delcayre et al., 1992, Amer. J. Physiol. 263:H1537-45. Animals are sacrificed with pentobarbital (0.3 mL), and intravenously heparinized (0.2 mL). The hearts are initially allowed to equilibrate for 15 min. The left ventricular balloon is then inflated to a volume that gives an end-diastolic pressure of 8 mm Hg. A left ventricular pressure-volume curve is constructed by incremental inflation of the balloon volume by 0.02 ml aliquots. Zero volume is defined as the point at which the left ventricular end-diastolic pressure is zero. On completion of the pressure-volume curve, the left ventricular balloon is deflated to set end-diastolic pressure back to 8 mmHg and the control period is pursued for 15 min, after check of coronary flow. Then the heart is arrested with 50 mL Celsior+ molecule to rest at 4° C. under a pressure of 60 cm H₂O. The heart is then removed and stored for 5 hours at 4° C. in plastic container filled with the same solution and surrounded with crushed ice.

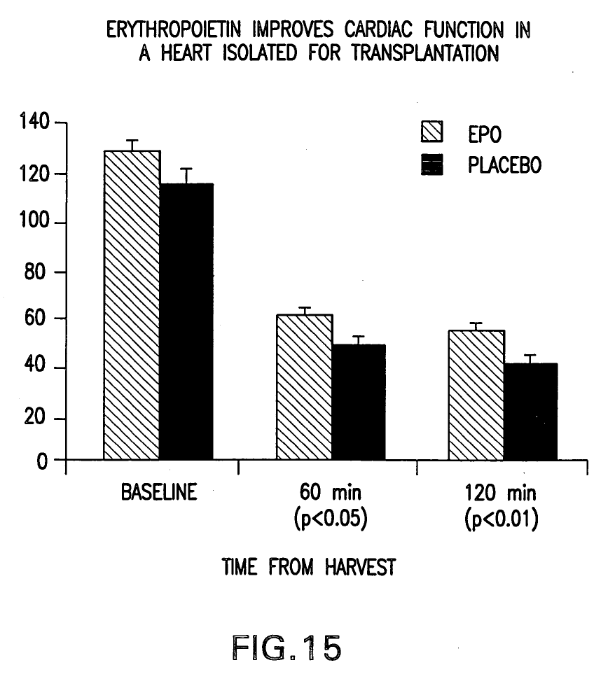

On completion of storage, the heart is transferred to a Langendorf apparatus. The balloon catheter is reinserted into the left ventricle and re-inflated to the same volume as during preischemic period. The heart is re-perfused for at least 2 hours at 37° C. The re-perfusion pressure is set at 50 cm H₂O for 15 min of re-flow and then back to 100 cm H₂O for the 2 next hours. Pacing (320 beats per minute) is re-instituted. Isovolumetric measurements of contractile indexes and diastolic pressure are taken in triplicate at 25, 45, 60, and 120 min of reperfusion. At this time point pressure volume curves are performed and coronary effluent during the 45 mn reperfusion collected to measure creatine kinase leakage. The two treatment groups are compared using an unpaired t-test, and a linear regression using the end-diastolic pressure data is used to design compliance curves. As shown in FIG. 15, significant improvement of left ventricular pressure developed occurs after treatment with erythropoietin, as well as improved volume-pressure curve, decrease of left diastolic ventricular pressure and decrease of creatine kinase leakage. Similar results would be expected from treatment with recombinant tissue protective cytokines of the present invention.

### 6.8. Example 8

### Erythropoietin Protects Myocardium from Ischemic Injury

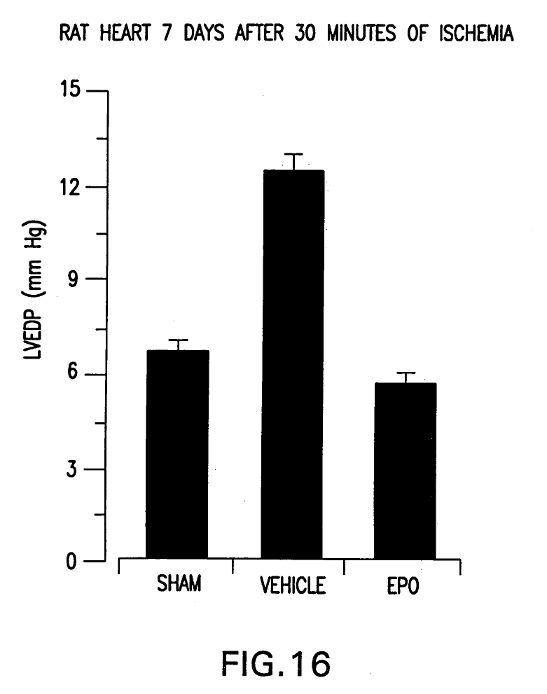

Adult male rats given recombinant human erythropoietin (5000 U/kg body weight) 24 hrs previously are anesthetized and prepared for coronary artery occlusion. An additional dose of erythropoietin is given at the start of the procedure and the left main coronary artery occluded for 30 minutes and then released. The same dose of erythropoietin is given daily for one week after treatment. The animals are then studied for cardiac function. As FIG. 16 illustrates, animals receiving a sham injection (saline) demonstrated a large increase in the left end diastolic pressure, indicative of a dilated, stiff heart secondary to myocardial infarction. In contradistinction, animals receiving erythropoietin suffered no decrement in cardiac function, compared to sham operated controls (difference significant at the p<0.01 level). Similar results would be expected from treatment with recombinant tissue protective cytokines of the present invention.

### 6.9. Example 9

### Protection of Retinal Ischemia by Peripherally-administered Erythropoietin.

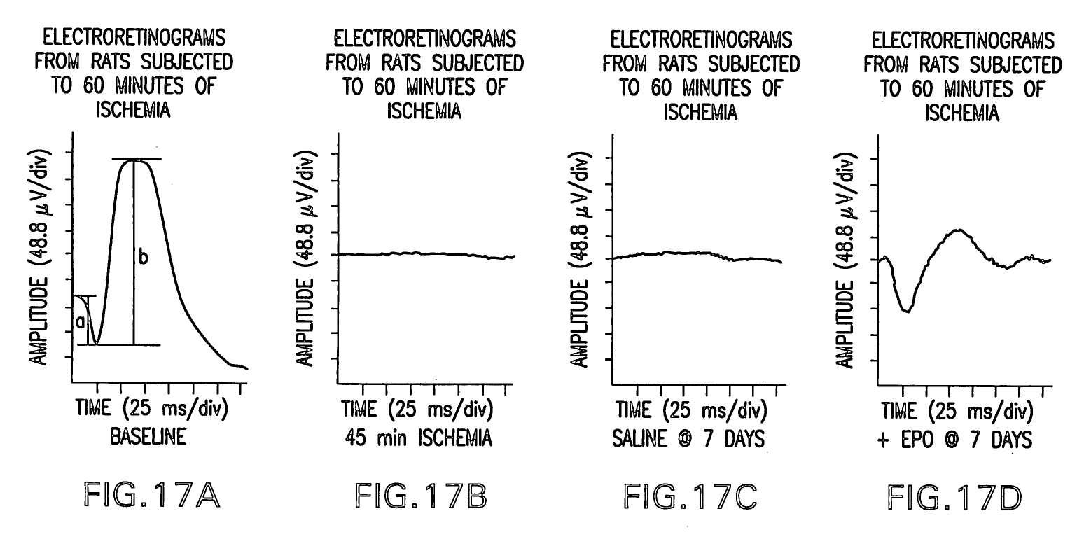

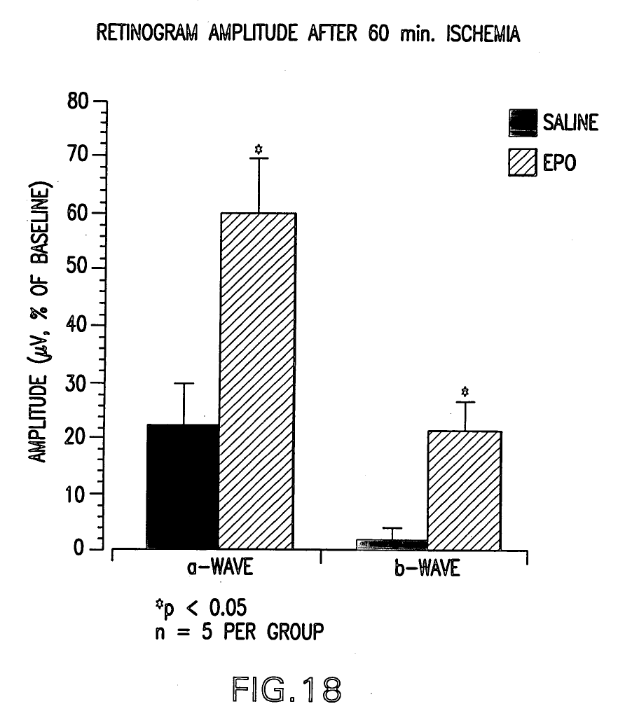

Retinal cells are very sensitive to ischemia such that many will die after 30 minutes of ischemic stress. Further, subacute or chronic ischemia underlies the deterioration of vision which accompanies a number of common human diseases, such as diabetes mellitus, glaucoma, and macular degeneration. At the present time there are no effective therapies to protect cells from ischemia. A tight endothelial barrier exists between the blood and the retina that excludes most large molecules. To test whether peripherally-administered erythropoietin will protect cells sensitive to ischemia, an acute, reversible glaucoma rat model was utilized as described by Rosenbaum et al. (1997; Vis. Res. 37:3443-51). Specifically, saline was injected into the anterior chamber of the eye of adult male rats to a pressure above systemic arterial pressure and maintained for 60 minutes. Animals were administered saline or 5000 U erythropoietin/kg body weight intraperitoneally 24 hours before the induction of ischemia, and continued as a daily dose for 3 additional days. Electroretinography was performed on dark-adapted rats 1 week after treatment. FIGS. 17-18 illustrate that the administration of erythropoietin is associated with good preservation of the electroretinogram (ERG) (FIG. 17, Panel D), in contrast to animals treated with saline alone (FIG. 17, Panel C), for which very little function remained. FIG. 18 compares the electroretinogram a- and b-wave amplitudes after 60 minutes ischemia for the erythropoietin-treated and saline-treated groups, and shows significant protection afforded by erythropoietin. Similar results are obtainable from treatment with recombinant tissue protective cytokines of the present invention.

### 6.10. Example 10

### Restorative Effects of Erythropoietin on Diminshed Cognitive Function Arising from Brain Injury

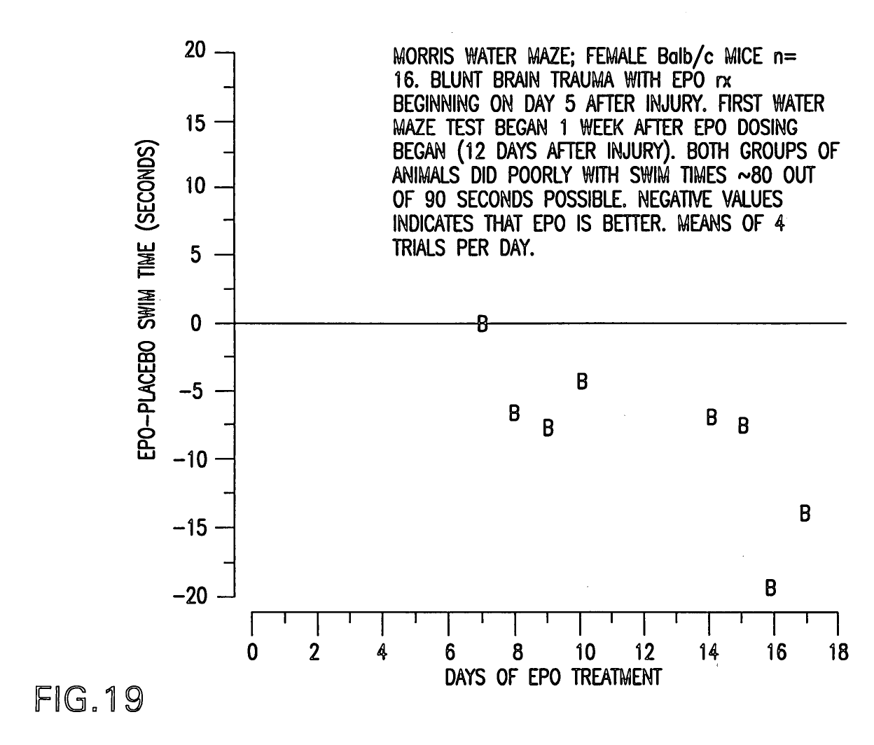

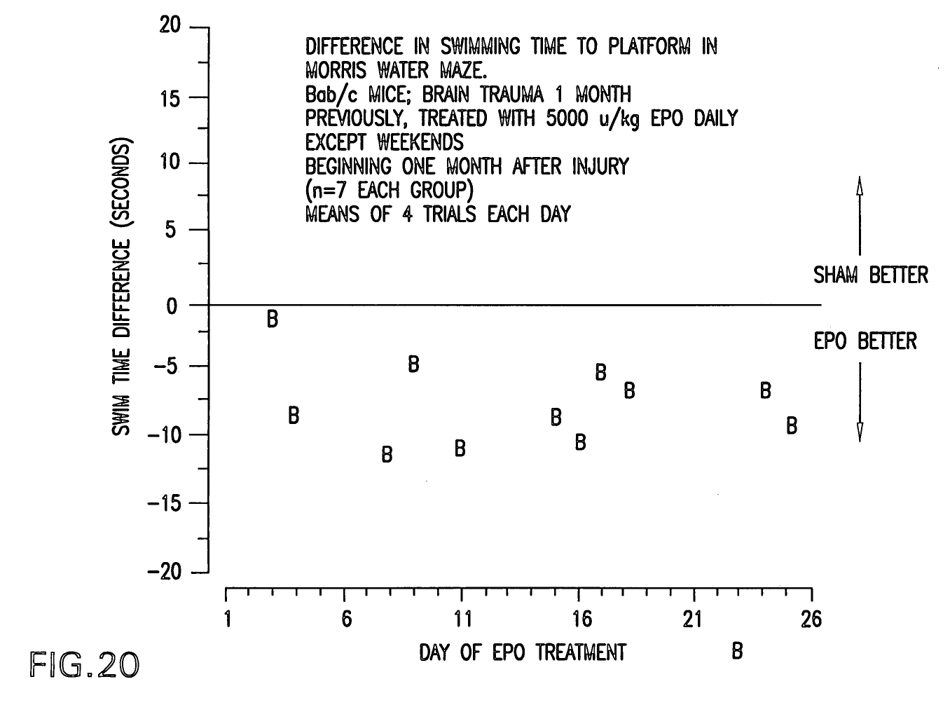

In a study to demonstrate the ability of erythropoietin to restore diminished cognitive function in mice after receiving brain trauma, female Balb/c mice were subject to blunt brain trauma as described in Brines et al. PNAS 2000, 97; 10295-10672 and five days later, daily erythropoietin administration of 5000 U/kg-bw intraperitoneally began. Twelve days after injury, animals were tested for cognitive function in the Morris water maze, with four trials per day. While both treated and untreated animals performed poorly in the test (with swim times of about 80 seconds out of a possible 90 seconds), FIG. 19 shows the results of the Morris Water maze test, with each group of mice, n=16, after blunt brain trauma with EPO administration beginning on day 5 after injury. The first test began 1 week after EPO dosing began (12 days after injury). Both groups of animals did poorly with swim times ˜80 out of 90 seconds possible. The erythropoietin-treated animals performed better (in this presentation, a negative value is better). Means of 4 trials per day were used. FIG. 19 shows that. Even if the initiation of erythropoietin treatment is delayed until 30 days after trauma (FIG. 20), restoration of cognitive function is also seen. In FIG. 20, the each group of mice, n=7, were treated with 5000 U/kg EPO daily except on weekends, beginning one month after injury. Means of trials were also 4 trials each day. Similar results are obtainable from treatment with recombinant tissue protective cytokines of the present invention.

### 6.11. Example 11

### Kainate Model

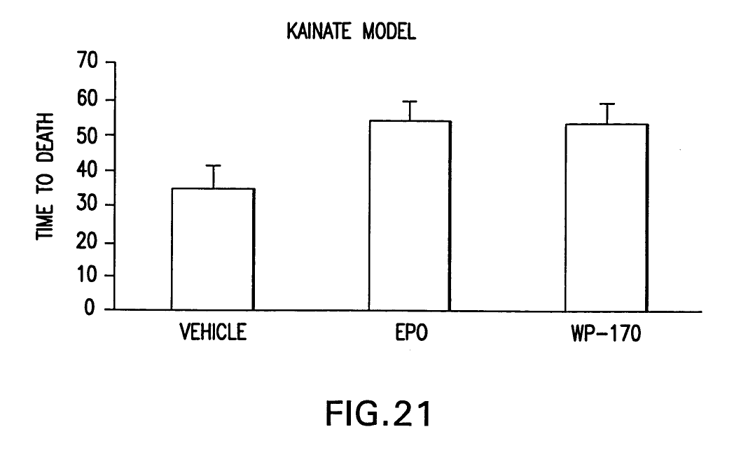

In the kainate neurotoxicity model, asialoerythropoietin was administered according to the protocol of Brines et al. Proc. Nat. Acad. Sci. U.S.A. 2000, 97; 10295-10672 at a dose of 5000 U/kg-bw given intraperitoneally 24 hours before the administration of 25 mg/kg kainate is shown to be as effective as erythropoietin, as shown by time to death (FIG. 21). Similar results are obtainable from treatment with tissue protective cytokines of the present invention.

### 6.12. Example 12

### Spinal Cord Injury Models

6.12.1. Rat Spinal Cord Compression Testing Erythropoietin and Tissue Protective Cytokines

Wistar rats (female) weighing 180-300 g were used in this study. The animals were fasted for 12 h before surgery, and were humanely restrained and anesthesized with an intraperitoneal injection of thiopental sodium (40 mg/kg-bw). After infiltration of the skin (bupivacaine 0.25%), a complete single level (T-3) laminectomy was performed through a 2 cm incision with the aid of a dissecting microscope. Traumatic spinal cord injury was induced by the extradural application of a temporary aneurysm clip exerting a 0.6 newton (65 grams) closing force on the spinal cord for 1 minute. After removal of the clip, the skin incision was closed and the animals allowed to recover fully from anethesia and returned to their cages. The rats were monitored continuously with bladder palpation at least twice daily until spontaneous voiding resumed.

Forty animals were randomly divided into five groups. Animals in the control group (I) (n=8) received normal saline (via intravenous injection) immediately after the incision is closed. Group (II; n=8) received rhEPO@ 16 micrograms/kg-bw iv; group (III) received an asialo tissue protective cytokine of the present invention (asialoerythropoietin) @ 16 micrograms/kg-bw iv, group (IV) received an asialo tissue protective cytokine @ 30 micrograms/kg-bw iv, and group (V) received an asialo tissue protective cytokine of the present invention (asialoerythropoietin) @ 30 micrograms/kg-bw; all as a single bolus intravenous injection immediately after removal of the aneurysm clip.

Motor neurological function of the rats will be evaluated by use of the locomotor rating scale of Basso et al. In this scale, animals are assigned a score ranging from 0 (no observable hindlimb movements) to 21 (normal gait). The rats will be tested for functional deficits at 1, 12, 24, 48, and 72 hours and then at 1 week after injury by the same examiner who is blind to the treatment each animal receives.

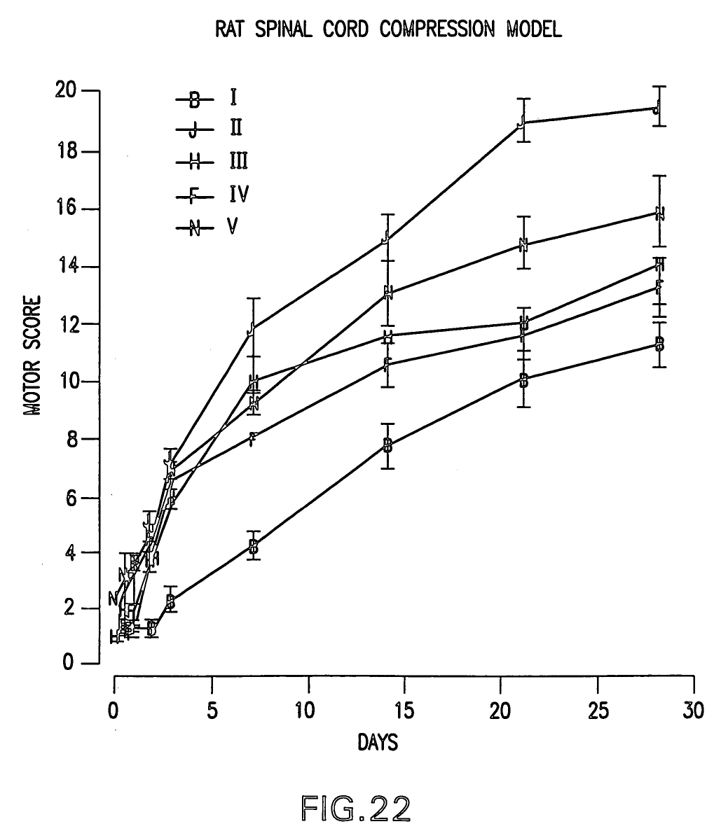

FIG. 22 is a graph demonstrating the locomotor ratings of the rats recovering from the spinal cord trauma over a period of thirty days. As can be seen from the graph, the rats that were given erythropoietin (group II) or tissue protective cytokines (groups III-V) recovered from the injury more readily and demonstrated better overall recovery from the injury than the control rats. Similar results would be expected from the therapeutic treatment with the recombinant tissue protective cytokines of the present invention.

In a second related study animals were injured in the same way. Forty animals were randomly divided into three groups. Animals in the control group (n=8) received normal saline (via intravenous injection) immediately after the incision is closed. The second group (n=8) received methylprednisolone @ 30 mg/kg per day ×3 then biweekly, a common therapeutic for spinal cord injury; the third group received an recombinant tissue protective cytokine, S100E, of the present invention at a dose of 10 ug/kg immediately following injury, all as a single bolus intravenous injection immediately after removal of the aneurysm clip.

Motor neurological function of the rats will be evaluated by use of the locomotor rating scale of Basso et al. In this scale, animals are assigned a score ranging from 0 (no observable hind limb movements) to 21 (normal gait). The rats will be tested for functional deficits at 1, 12, 24 , 48, and 72 hours and then at 1 week after injury by the same examiner who is blind to the treatment each animal receives.

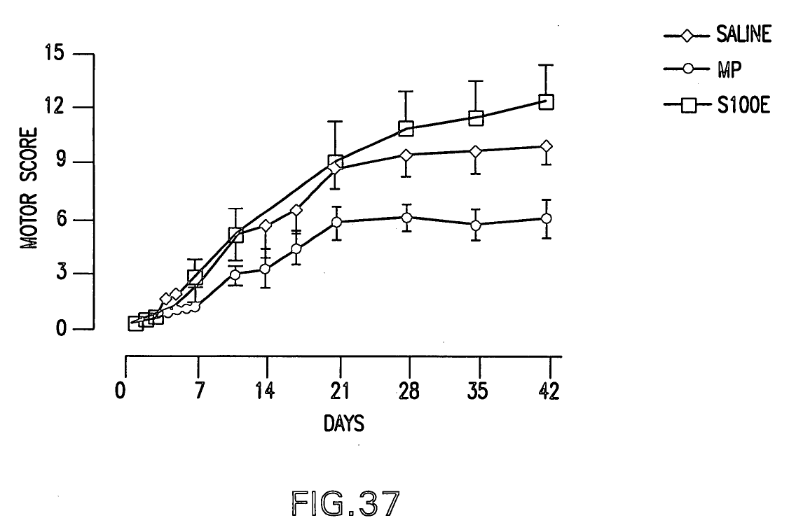

FIG. 37 is a graph demonstrating the locomotor ratings of the rats recovering from the spinal cord trauma over a period of forty-two days. As can be seen from the graph, the rats that were given S100E recovered from the injury more readily and demonstrated better overall recovery from the injury than the control rats and rats administered methylprednisolone.

6.12.2. Rabbit Spinal Cord Ischemia Testing Erythropoietin and a Tissue Protective Cytokine.

Thirty-six New Zealand White rabbits (8-12 months old, male) weighing 1.5-2.5 kg were used in this study. The animals were fasted for 12 hours and humanely restrained. Anesthesia induction was via 3% halothane in 100% oxygen and maintained with 0.5-1.5% halothane in a mixture of 50% oxygen and 50% air. An intravenous catheter (22 gauge) was placed in the left ear vein. Ringers lactate was infused at a rate of 4 ml/kg body weight (bw) per hour during the surgical procedure. Preoperatively, cefazoline 10 mg/kg-bw was administered intravenously for prophylaxis of infection. The animals were placed in the right lateral decubitus position, the skin prepared with povidone iodine, infiltrated with bupivacaine (0.25%) and a flank skin incision was made parallel to the spine at the 12th costal level. After incision of the skin and subcutaneous thoracolumbar fascia, the longissimus lumborum and iliocostalis lumborum muscles were retracted. The abdominal aorta was exposed via a left retroperitoneal approach and mobilized just inferior to the left renal artery. A piece of PE-60 tubing was looped around the aorta immediately distal to the left renal artery and both ends passed through a larger rubber tube. By pulling on the PE tubing, the aorta was non-traumatically occluded for 20 minutes. Heparin (400 IU) was administered as an intravenous bolus before aortic occlusion. After 20 minutes of occlusion, the tube and catheter were removed, the incision was closed and the animals were monitored until full recovery and then were serially assessed for neurological function.

Thirty-six animals were randomly divided into six groups. In a control group (1), animals (n=6) received normal saline intravenously immediately after release of aortic occlusion. Group (II) received rhEPO @ 6.5 microgram/kg-bw; group (III) received a tissue protective cytokine (carbamylated erythropoietin) @ 6.5 microgram/kg-bw; group (IV) received another tissue protective cytokine (asialoerythropoietin) @ 6.5 microgram/kg-bw; group (V) received the same tissue protective cytokine as group (IV) but @ 20 microgram/kg-bw; and group (VI) received yet another tissue protective cytokine (asialocarbamylatederythropoietin) @ 20 microgram/kg-bw all intravenously immediately after reperfusion (n=6 for each group).

Motor function was assessed according to the criteria of Drummond and Moore by an investigator blind to the treatment at 1, 24 and 48 h after reperfusion. A score of 0 to 4 was assigned to each animal as follows: 0=paraplegic with no evident lower extremity motor function; 1=poor lower extremity motor function, weak antigravity movement only; 2=moderate lower extremity function with good antigravity strength, but inability to draw legs under body; 3=excellent motor function with the ability to draw legs under body and hop, but not normally; 4=normal motor function. The urinary bladder was evacuated manually in paraplegic animals twice a day.

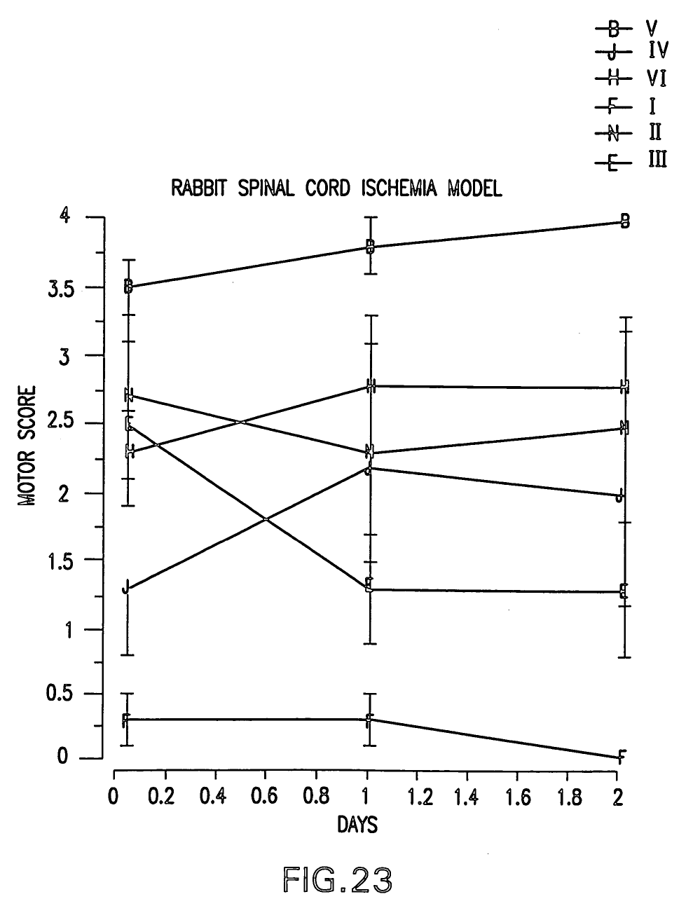

FIG. 23 is a graph plotting motor function of the recovering rabbits. The graph demonstrates that even over a period of only two days erythropoietin and the tissue protective cytokines of the present invention permit the rabbits to recover more fully from the spinal cord injury. Similar results would be expected from the therapeutic treatment with the recombinant tissue protective cytokines of the present invention.

### 6.13. Example 13

### Anti-Inflammatory Affects ofErythropoietin

In-Vivo Studies:

MCAO in Rats

Male Crl:CD(SD)BR rats weighing 250-280 g were obtained from Charles River, Calco, Italy. Surgery was performed on these rats in accordance with the teachings of Brines, M. L., Ghezzi, P., Keenan, S., Agnello, D., de Lanerolle, N. C., Cerami, C., Itri, L. M., and Cerami, A. 2000 Erythropoietin crosses the blood-brain barrier to protect against experimental brain injury, Proc Natl Acad Sci USA 97:10526-10531. Briefly, the rats were anesthetized with chloral hydrate (400 mg/kg-bw, i.p.), the carotid arteries were visualized, and the right carotid was occluded by two sutures and cut. A burr hole adjacent and rostral to the right orbit allowed visualization of the MCA, which was cauterized distal to the rhinal artery. To produce a penumbra (borderzone) surrounding this fixed MCA lesion, the contralateral carotid artery was occluded for 1 hour by using traction provided by a fine forceps and then re-opened. PBS or rhEPO (5,000 U/kg-bw, i.p.; previously shown to be protective in this model (1)) were administered immediately after the MCAO. When indicated, TNF and IL-6 were quantified in brain cortex homogenates as previously described (8). MCP-1 was measured in the homogenates using a commercially available ELISA kit (biosource, Camarillo, Calif.).

Twenty-four hours after MCAO, the rats were anesthetized as described above and transcardially perfuised with 100 ml saline followed by 250 ml of sodium phosphate buffered 4% paraformaldehyde solution. Brains were rapidly removed, fixed in sodium phosphate buffered 4% paraformaldehyde solution for two hours, transferred to 20% sucrose solution in PBS overnight, then in 30% sucrose solution until they sank and were then frozen in 2-methylbutane at −45° C. Sections (30 μm) were cut on a cryostat (HM 500 OM, Microm) at −20° C. in the transverse plane through the brain and selected every fifth section for histochemistry against the different antigens, or hematoxylin-eosin staining. Free floating sections were processed for immunoreactivity both with anti-glial fibrillary acid protein (GFAP) mouse monoclonal antibody (1:250, Boehringher Mannheim, Monza, Italy) and with anti-cd11b (MRC OX-42) mouse monoclonal antibody (1:50, Serotec, UK), according to the protocols described by Houser et al. and the manufacturer's protocol respectively. All sections were mounted for light microscopy in saline on coated slides, dehydrated through graded alcohols, fixed in xylene and coverslipped using DPX mountant (BDH, Poole, UK). Adjacent sections were stained with hematoxylin-eosin as described (10).

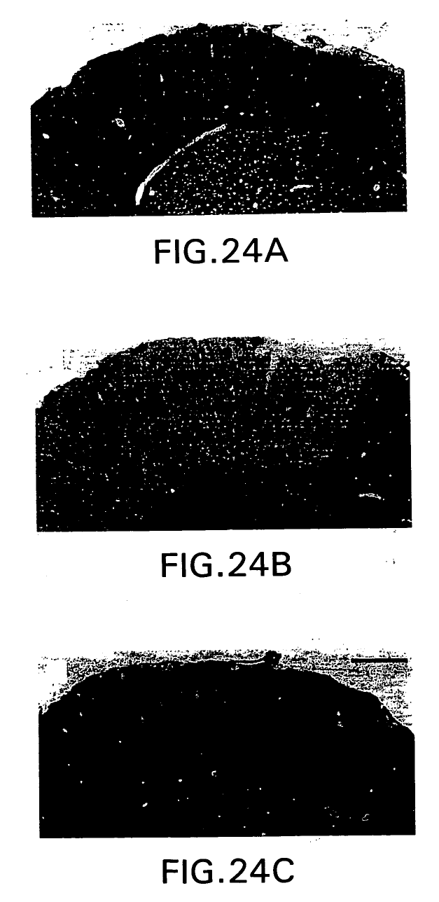

FIG. 24 shows a coronal section of the brain cortical layer stained by hematoxilyn and eosin. Control rat (A), ischemic rat treated with PBS (B), ischemic rat treated with rhEPO (5,000 U/kg-bw, i.p., immediately following MCAO) (C). Section B shows a marked decrease in tissue staining consistent with inflammation, accompanied by a loss of neuronal component compared to the control (A). Systemic rhEPO administration largely reduces the ischemic damage localizing the cell death or injury in a restricted area (C). (Magnification 2.5×. Size bar=800 μm.)

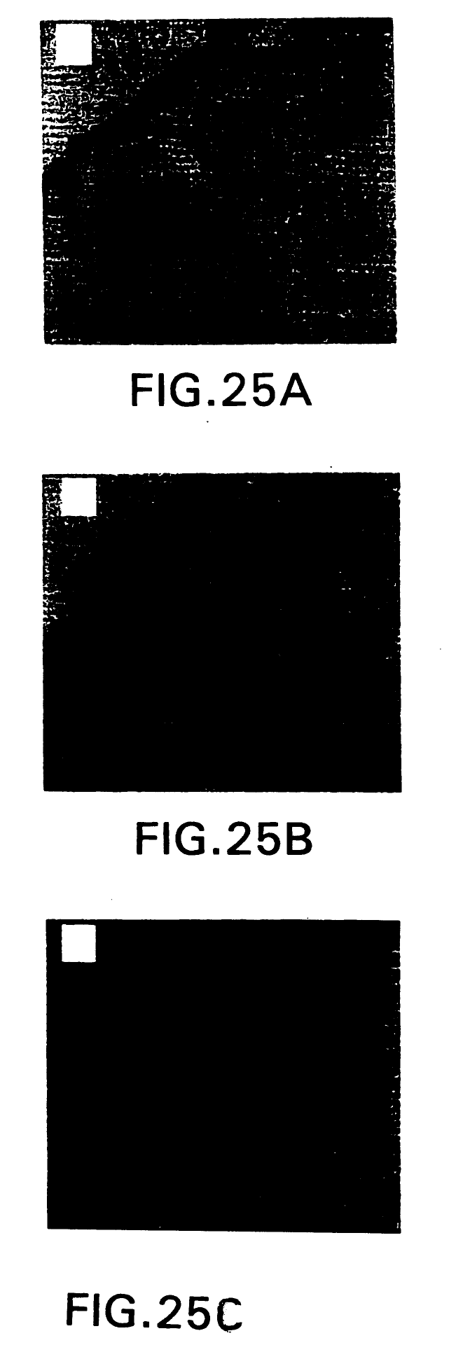

FIG. 25 shows coronal sections of frontal cortex adjacent to the region of infarction stained by GFAP antibody. Control rat (A), ischemic rat treated with PBS (B), and ischemic rat treated with rhEPO (C). Activated astrocytes are visualized by their GFAP-positive processes (Panel B). Note the marked reduction in number as well as staining intensity in a representative rhEPO-treated animal (Panel C). (Magnification 10×. Size bar=200 μm.)

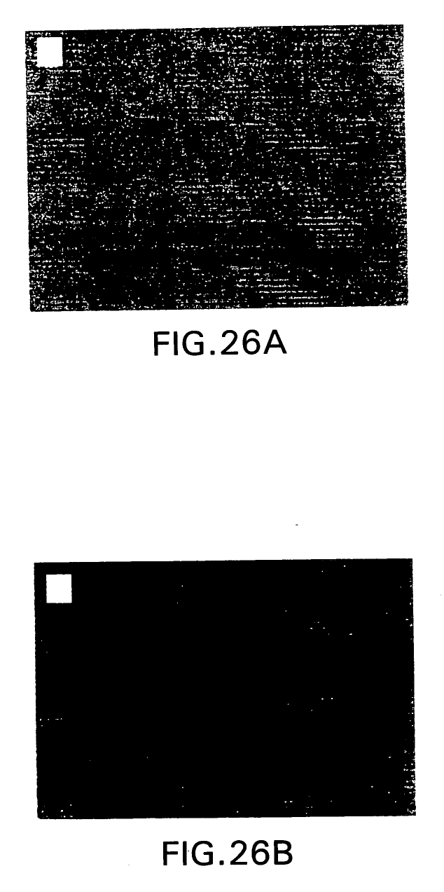

FIG. 26 shows coronal sections of brain cortical layer stained by OX-42 antibody. Ischemic rat treated with PBS (A), and ischemic rat treated with rhEPO (B). In the ischemic cerebral hemisphere, the cellular staining is especially prominent around the infarcted tissue in both treatment groups, but it is much denser and extends further in the saline treated group. (Magnification 20×; Size bar=100 μm).

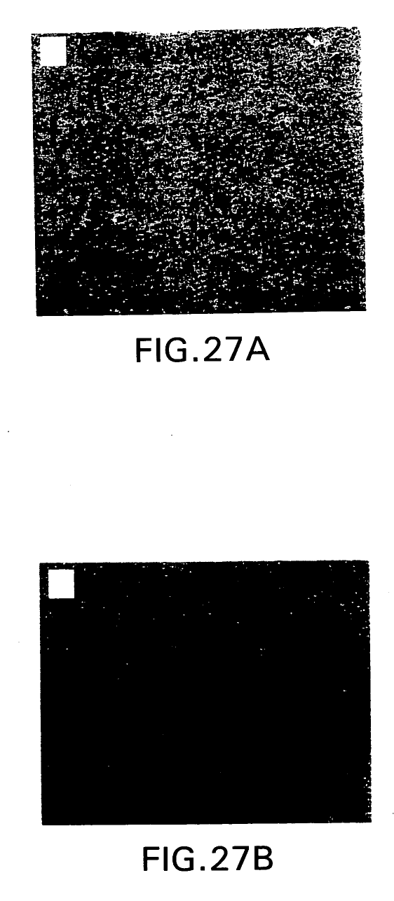

FIG. 27 shows coronal sections of brain cortical layer adjacent to the region of infarction stained by OX-42 antibody. A much higher density of mononuclear inflammatory cells are observed in the tissue from an ischemic rat treated with PBS (A) compared to an ischemic rat treated with rhEPO (B). The infiltrating leukocytes, with typical round shape, potentially will extend the volume of infarction. (Magnification 10×; Size bar=200 μm)

Similar results would be expected from the therapeutic treatment with the recombinant tissue protective cytokines of the present invention.

Acute Experimental Allergic Encephalomyelitis (EAE) in Lewis rats

Female Lewis rats, 6-8 weeks of age, were purchased from Charles River (Calco, Italy). EAE was induced in rats by injecting 50 μg of guinea pig MBP (Sigma, St. Louis, Mo.) in water emulsified in equal volumes of complete Freund's adjuvant (CFA, Sigma) additioned with 7 mg/ml of heat-killed M. tuberculosis H37Ra (Difco, Detroit, Mich.) in a final volume of 100 μ under light ether anesthesia into both hind footpads. 1. Rats were examined in a blinded fashion for signs of EAE and scored as follows: 0, no disease; 1, flaccid tail; 2, ataxia; 3, complete hind limb paralysis with urinary incontinence. Starting from day 3 after immunization, rats were given r-Hu-EPO (EPOetin alfa, Procrit, Ortho Biotech, Raritan, N.J.) intraperitoneally (i.p.) once a day at the indicated doses, in PBS. Since the clinical-grade EPO contained human serum albumin, control animals were always given PBS containing an identical amount of human serum albumin. Daily administration of 5,000 U/kg-bw of EPO increased the hematocrit by 30%. When indicated, rats were injected s.c. once a day from day 3 until day 18 with 1.3 mg/kg-bw dexamethasone (DEX) phosphate disodium salt (Sigma) corresponding to 1 mg/kg-bw of DEX, dissolved in PBS. When indicated, TNF and IL-6 were quantified in brain and spinal cord homogenates as previously described [Agnello, 2000 #10].

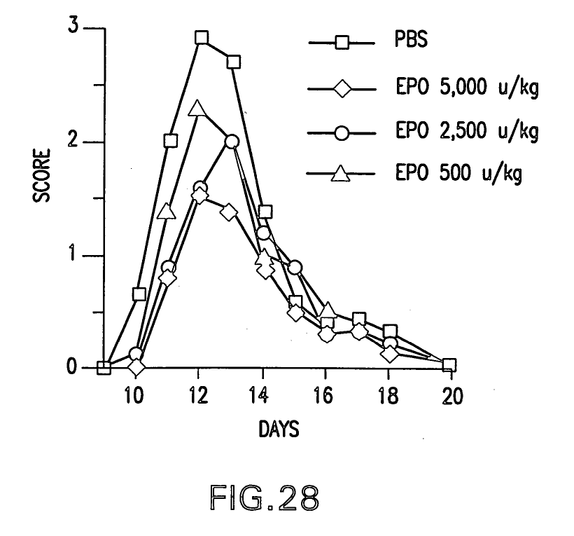

FIG. 28 shows the protective effect on the clinical signs of EAE of different doses of EPO, given from day 3 after immunization with MBP until day 18. EPO, in a dose-dependent fashion, delayed the onset of disease and decreased disease severity, as summarized in Table 1, but did not delay the time to greatest severity. As shown in this table, EPO at the doses of 2,500 and 5,000 U/kg-bw significantly decreased the mean cumulative score.

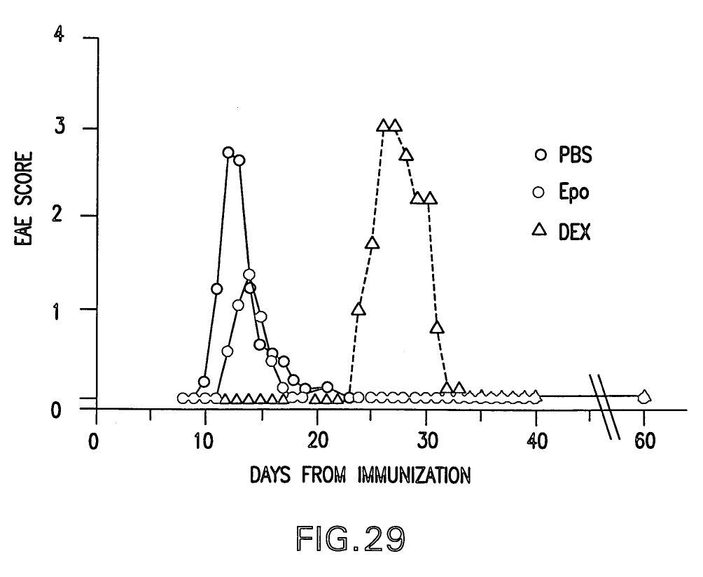

In experiments where treatment of EPO was discontinued after the disease regressed and the rats were monitored up to two months, no relapse was observed, in contrast with DEX which induces an exacerbation of disease after suspending its administration (FIG. 29). Similar results would be expected from the therapeutic treatment with the recombinant tissue protective cytokines of the present invention.

In Vitro Studies:

Primary cultures of glial cells were prepared from newborn Sprague-Dawley rats 1-2 days old. Cerebral hemispheres were freed from the meninges and mechanically disrupted. Cells were dispersed in a solution of trypsin 2.5% and DNAase 1%, filtered through a 100 μm nylon mesh and plated (140,000 cells per 35 mm dish) in Eagle's minimum essential medium supplemented with 10% fecal calf serum, 0.6% glucose, streptomycin (0.1 mg/ml) and penicillin (100 U/ml). Glial cultures were fed twice a week and grown at 37° C. in a humidified incubator with 5% CO2. All experiments were performed on 2-3 week-old glial cell cultures with 97% astrocytes and 3% microglia, as assessed by immunochemistry o of GFAP and Griffonia simplicifolia isolectin B4. Neuronal cultures were established from the hippocampus of 18-day rat fetuses. Brains were removed and freed from meninges and the hippocampus was isolated. Cells were dispersed by incubation for 15-20 min at 37° C. in a 2.5% trypsin solution followed by titration. The cell suspension was diluted in the medium used for glial cells and plated onto polyornithine-coated coverslips at a density of 160,000 cells per coverslip. The day after plating, coverslips were transferred to dishes containing a glial monolayer in neuron maintenance medium (Dulbecco's modified Eagle's medium and Ham's nutrient mix F12 supplemented with 5 μg/ml insulin, 100 μg/ml transferrin, 100 μg/ml putrescine, 30 nM Na selenite, 20 nM progesterone and penicillin 100 U/ml) supplemented with cytosine arabinoside 5 μM. Coverslips were inverted so that the hippocampal neurons faced the glia monolayer. Paraffin dots adhering to the coverslips supported them above the glia, creating a narrow gap that prevented the two cell types from contacting each other but allowed the diffusion of soluble substances. These culture conditions allowed the growth of differentiated neuronal cultures with >98% homogeneity, as assessed by immunochemistry of microtubule-associated protein 2 and GFAP. Cells were then treated for 24 hours with 1 μM Trimethyl tin (TMT), in the presence or absence of rhEPO (10 U/ml), the supernatants used for TNF assay and cellular viability evaluated as described below. When indicated, glial cells were cultured in the presence of LPS for 24 hours, with or without rhEPO, and TNF measured in the cultured supernatants. Cell viability was measured by the 3-(4,5-dimethyl-thiazol-2-yl)-2,5-diphenyltetrazolium bromide (MTT) assay. Denizot, F., and Lang, R. 1986. Rapid calorimetric assay for cell growth and survival. Modifications to the tetrazolium dye procedure giving improved sensitivity and reliability. J Immunol Methods 89:271-277. Briefly, MTT tetrazolium salt was dissolved in serum-free medium to a final concentration of 0.75 mg/ml and added to the cells at the end of the treatment for 3 h at 37° C. The medium was then removed and the formazan was extracted with IN HCI:isopropanol (1:24). Absorbance at 560 nm was read on a microplate reader.

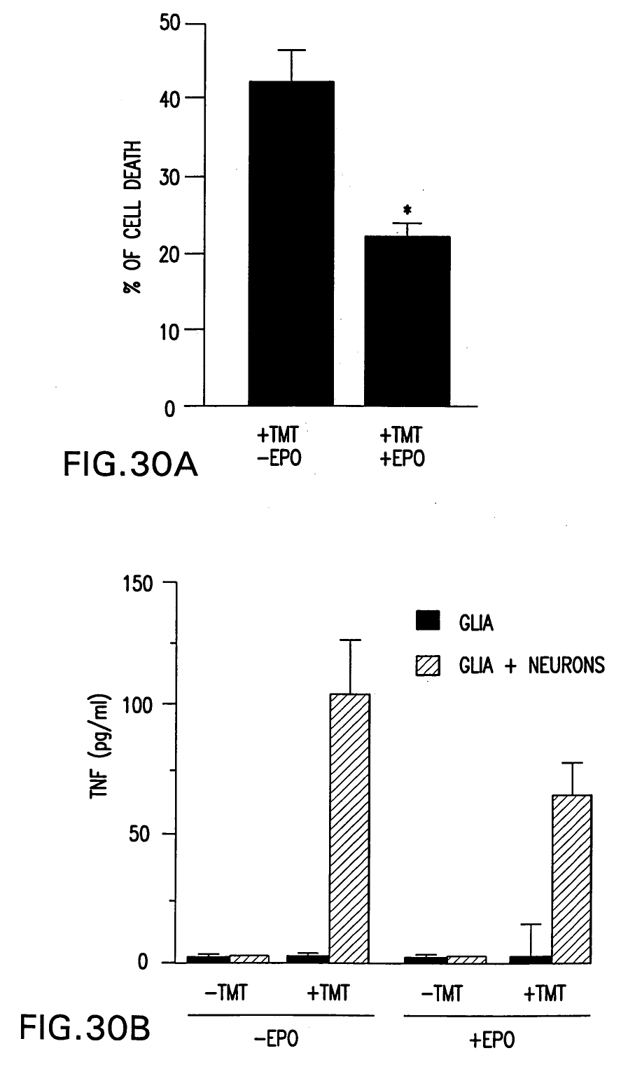

FIG. 30 shows that rhEPO prevents neuronal death-induced TNF production in mixed neuron-glia cultures. Panel A: Percentage of neural cell death induced by TMT 1 μM without or with treatment with rhEPO (10 U/ml). Panel B: Release of TNF-α from glial cells exposed to TMT 1 μM in the presence (hatched bars) or absence (filled bars) of neurons, with or without rhEPO (10 U/ml). Similar results would be expected from the therapeutic treatment with the recombinant tissue protective cytokines of the present invention.

### 6.14. Example 14

### NMDA Induced Call Death Assay

Excitotoxicity can be defined as the excessive activation of glutamate receptors, such as the N-methyl-D-aspartate (NMDA) receptor. The NMDA receptor exhibits increased activity in response to ischemia and other traumas (Fauci et al., 1998, Harrison's Principles of Internal Medicine), (Nishizawa, 2001, Life Sci. 69, 369-381), (White et al., 2000, J. Neurol. Sci. 179, 1-33). Thus, the assay serves as a model for assesing a compounds effect on cell injury and death.

Protocol of NMDA excitotoxicity in primary hippocampal neurons

Primary hippocampal neuronal cultures were prepared from new born mice (less than 24 hours old) essentially as previously described by Krohn et al. (1998). Briefly, the hippocampi were dissected out in DMEM containing 0.02% BSA. The tissue was transferred to DMEM containing 0.1 % papain and incubated for 20 minutes at 37° C. The digestion was stopped by aspiration of the papain containing medium and addition of MEMII and the hippocampal cells were dissociated by tituration with a 1000 μl pipet tip. The tissue pieces were allowed to settle and the supernatant, containing single cells, was transferred into MEMII containing 1% trypsin inhibitor (type 11-0) and 1% BSA. The tituration-step was repeated three times before the single cells were centrifuged at 600U/minute for 10 minutes and resuspended in growth medium (MEMII, 20 mM D-glucose, 100 U/ml penicillin, 100 μg streptomycin, 2 mM L-glutamine, 10% Nu-serum (bovine), 2% B27 supplement, 26.2 mM NaHCO3). Cells from 10 hippocampi were used to seed one 24 well plate. One day after seeding, the cells were treated with cytosine-arabino-furanoside (1 μM). On day two, the medium was changed and cytosine-arabino-furanoside (1 μM) was added.

Excitotoxicity Assay

Twelve day old cultures were pre-incubated with test compound (vehicle, R103E, R150E, or EPO) at 5 nM for 24 hours. On day 13, the medium was removed from the cells and kept while the cultures were challenged with 300 μM NMDA for 5 minutes at room temperature. After the excitotoxic insult, the pre-conditioned medium was returned to the cultures and the injury was quantified by trypan blue exclusion after another 24 hours of incubation. Approximately 300 neurons were counted per condition in at least four separate wells and the experiments were repeated at least twice (Krohn, A. J., Preis, E. and Prehn, J. H. M. (1998) J. Neurosci. 18(20):8186-8197).

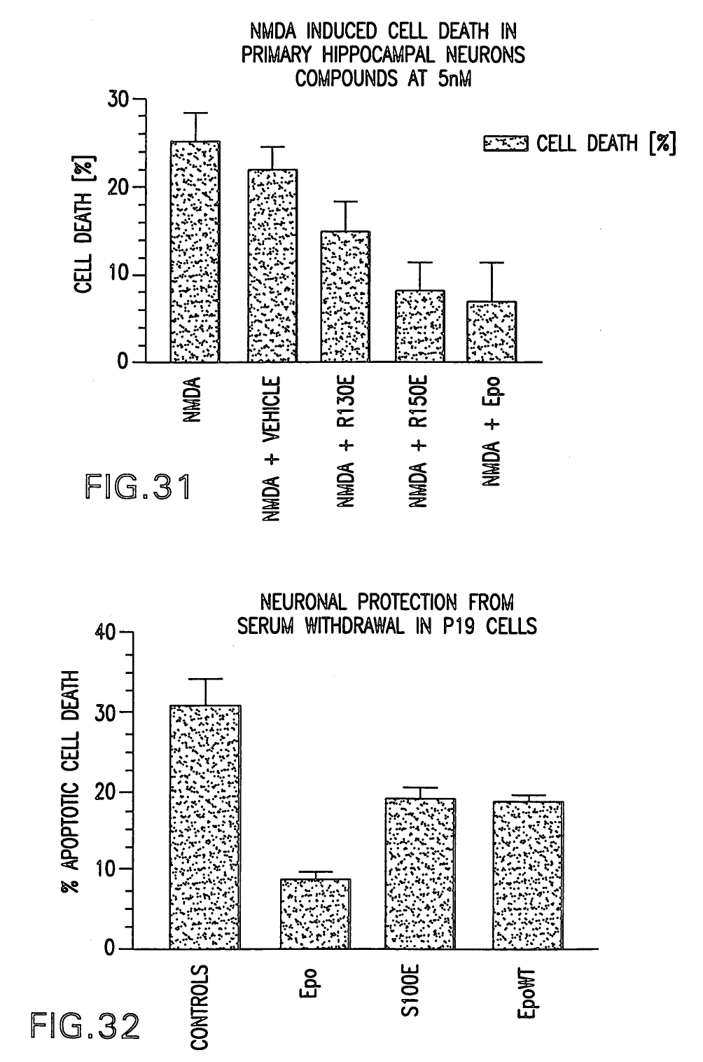

FIG. 31 shows that human erythropoietin and recombinant tissue protective cytokines R130E and R150E effectively reduce cell death induced by NMDA when added to the primary hippocampal neuron cell cultures prior to NMDA treatment. Cells treated with R103E (5 nM) exhibited significantly less cell death in comparison to vehicle control cells (p=0.01). Cells treated with R103E (5 μM) exhibited significantly less cell death in comparison to vehicle control cells (p=0.01). Cells treated with R150E (5 nM) exhibited approximately a 20% decrease in cell death in comparison to solvent control cells (p=0.001). Statistics: ANOVA plus Tukey's post-hoc test.

### 6.15. Example 15

### Neuronal Protection of Serum withdrawal in P19 Cells

To examine the neuronal protective effect of the recombinant tissue protective cytokines of the invention, withdrawal of serum from PC19 cell cultures was used as a model. The clone P19S1801A1 was kindly provided by Dr. W. H. Fischer. The cells were maintained in Dulbecco's Modified Eagles Medium (DMEM) supplemented with 2 mM L glutamine, 100 U/ml penicillin G, 100 μg/ml streptomycin sulfate and 10% fetal calf serum (FCS; all from Gibco, Paisley, Scotland, UK), containing 1.2 g/l NaHCO₃, 10 mM Hepes buffer (Carlo Erba, Milano, Italy), hereafter referred to as complete medium, in a humidified incubator under an atmosphere of 7% CO₂ in air. Serum free medium (N2) has the same constituents as above with the deletion of serum, and the addition of the following: 5 μg/ml of insulin, 100 μg/ml of transferrin, 20 nM progesterone, 100 μM putrescine and 30 nM Na₂SeO₃ (all from Sigma). For the death experiments, cells were dissociated with 10% pancreatin (Gibco), washed once with complete medium, twice with N2 medium and plated, unless otherwise indicated, plated in 25 cm² tissue culture flasks (Falcon Becton Dickinson, Lincoln Park, N.J.) at a final density of 104 cells/cm² in 5 ml of serum-free medium. L acetylcarnitine (100 μM) is taken as a positive control, that confers protection, reducing by 50% the percentage of apoptotic nuclei 24 h after serum deprivation. Twenty-four h after serum deprivation, cells were detached by tapping on the flask (without trypsin) seeded on microscope slides by cytospin centrifugation (Shandon Southern, USA) at 600 rpm for 10 min, and fixed in Carnoy solution (methanol:acetic acid, 3:1) for 10 min, stained with Hoechst 33258 (0.1 μg/ml PBS) for 1 h at 37° C., washed with tap water for 15 min, air dried and mounted. Slides were observed with a fluorescence microscope (Zeiss, Germany) at an excitation wavelength of 365 nm. The percentage of apoptotic nuclei was determined by counting in blind a total of 100 cells in at least 5 determinations.

P19 cells were pre-incubated with 3 nM Epo or recombinant tissue protective cytokine S100E for 24h. This treatment resulted in significant (p<0.001) protection from apoptosis triggered by serum withdrawal. Data are means from triplicate determinations within one experiment. The experiment was performed twice with similar results.

FIG. 32 shows neuronal protection from serum withdrawal in P19 cells. The percent of apaptotic cells decreased for cells pretreated with Epo, EpoWT, and recombinant tissue protective cytokine S100E. Cells treated with Epo exhibited approximately a 20% decrease in apoptotic cell death in comparison to untreated control cells. Cells treated with EpoWT and S100E both exhibited approximately a 10% decrease in apoptotic cell death in comparison to untreated control cells.

### 6.16. Example 16

### NGF withdrawal in Differentiated PC12 Cells

To examine the neuronal protective effect of the recombinant tissue protective cytokines of the invention, withdrawal of NGF in differentiated PC12 cells was used as a model. The assay is a well-established model of apoptotis. This PC12 rat cell line was derived from an adrenal medullary phaeochromocytoma and can be differentiated into neuronal-like cells in the presence of NGF (Masuda et al., 1993, J Biol Chem 268, 11208-11216). The PC12 cell line is a neuroendocrine cell line, which in the presence of NGF can be differentiated to express a neuronal-like phenotype (Vaudry et al., 2002, Science 296, 1648-1649). Once the cells are are fully differentiated they become NGF-dependent and withdrawal of NGF induces apoptosis.

PC12 cells were maintained in Dulbecco's modified Eagle's medium (DMEM) supplemented with 10% heat inactivated horse serum, 5% heat inactivated fetal bovin serum, 1% sodium-pyruvate and 1% penicillin-streptomycin (P/S) (Invitrogen, Carlsbad, USA).

For experiments, cells were differentiated for 7 days in collagen G-coated 48 well plates at a density of 24,000 cells/well in DMEM supplemented with 1% heat inactivated horse serum, 1% sodium-pyruvate, 1% P/S and 100 ng/ml NGF (7S nerve growth factor, mouse submaxillary glands, purchased from Calbiochem, Cat. No.480354) with medium changed every 2-3 days. At day 6, the Epo mutant at amino acid 100 (=SIOOE) was added to the cells in the indicated concentrations for 24 hours, after which medium was replaced with RPM11640, 1% P/S, to remove NGF from all cells. S100E was re-added, as was NGF (100 ng/ml) as positive control (+NGF). After 24 h, viability was measured by a tetrazolium (MTT)-reduction assay.

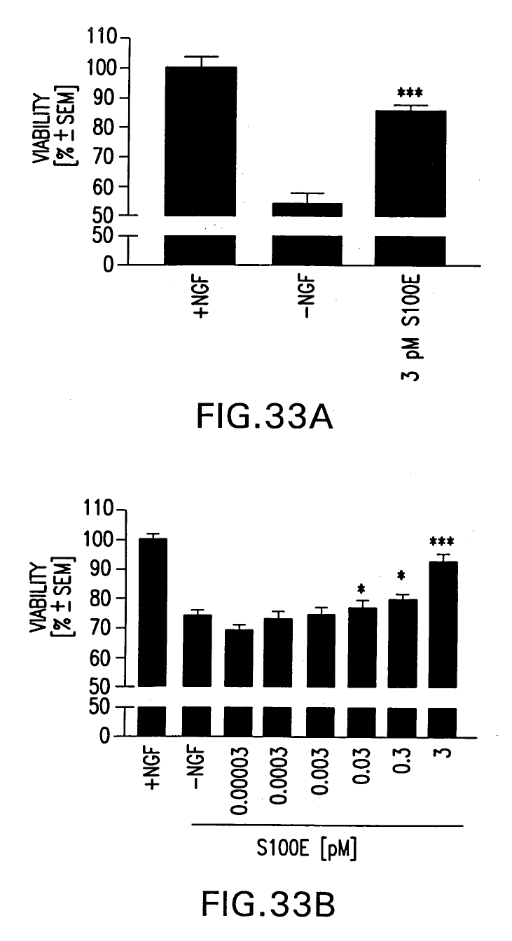

FIGS. 33A and 33B Show the effect of pre-incubation with S100E in differentiated PC12 cells submitted to NGF withdrawal in two independent experiments. Differentiated PC12 cells were pre-treated with S100E at the indicated concentrations for 24 h, FIG. 33A (3 pM) FIG. 33B (0.00003 pM-3 pM). Viability was measured in the MTT assay. NGF (100 ng/ml) was used as a positive control and NGF-free medium (−NGF) as a negative control. Data presented in FIG. 33 are presented as % viability of positive control (+NGF) (n=8 in both experiments). There is a statistically significant increase in viability of S100E treated cells compared to negative control cells (−NGF) by use of one-way ANOVA and Bonferroni post-hoc test. ***p<0.001, *p<0.05. The effects observed with S100E were similar to those of Epo in this test system with respect to potency and efficacy.

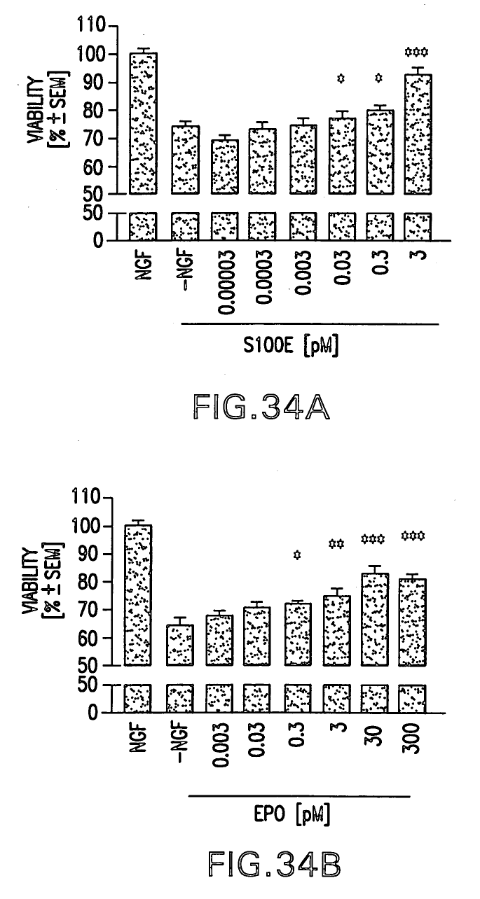

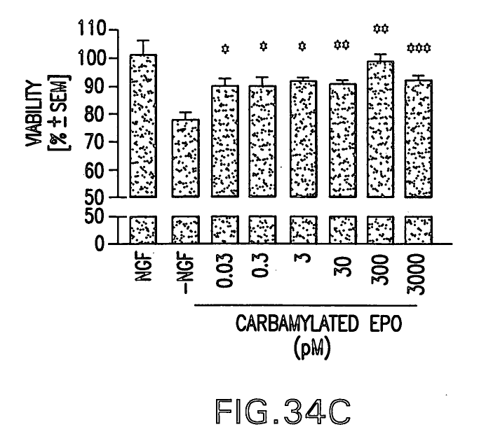

FIG. 34 Shows the effect of pre-incubation with Epo in differentiated PC12 cells submitted to NGF withdrawal. Differentiated PC 12 cells were pre-treated with Epo, S100E, or carbamylated Epo (30 pM-30 nM) for 24 h. The chemically modified Epo molecule, AA24496, has a 10000 times lower activity than EPO in the UT-7 cell assay. Viability was measured in the MTT assay. NGF (100 ng/ml) was used as a positive control and NGF-free medium (−NGF) as a negative control.

### 6.17. Example 17

### EPO Bio-Assay UT-7 Cell Proliferation

UT-7 is a leukaemia Epo-dependent cell line used for the determination of the erythroid effect of recombinant tissue protective cytokine such as K45D. The UT-7 cells (Deutsche Sammlung von Mikroorganismen und Zellkulturen (DSMZ), Cat. No. ACC 363) were normally grown in the presence of 10% FBS and 5 ng/ml Epo. The proliferation/survival (=viability increase) response of the cells exposed to Epo is mediated by the classical peripheral-type Epo receptor. The proliferation response is a quantitative measure of and correlates with the capacity of Epo-variants to stimulate the classical Epo receptor.

Methods for UT-7 Cell Viability Assay

The human leukemia cell line UT7 was made Epo dependent, and the proliferative response to added Epo/recombinant tissue protective cytokines was used as a measurement for their biological activity. On day one of the assay the cells were transferred to fresh complete RPMI 1640 media with 10% serum containing Epo (5 ng/ml) (10% donor calf serum, 4 mM L-glutamine, supplemented with 5 ng/ml of rhuEPO). The cells were grown in the 75 cm² flasks with 20 ml of culture/flask. On day two of the assay the cells were transferred from the flask(s) into a 50-ml conical tube and centrifuge at 1,000 rpm for 5 minutes at room temperature. The old media was discard and the cells were washed two times with 10 ml of starvation media (3% donor calf serum, 4 mM L-glutamine). The cells were re-suspended in starvation media, using pipet action up and down to obtain a single cell suspension. To determine the cell density, the re-suspended cells were diluted with starvation media to a density of 4×10⁵ cells/ml with a total culture volume of 10 ml and placed in a 25 cm² flask. The mixture was incubated for 4 h in a humidified incubator with 5% CO₂ at 37° C. During the last hour of incubation, a 96 well plate was prepared. At the end of the 4-hour incubation, the cell cultures were removed from the incubator, and the cells were transferred from a flask to a 50-ml conical tube. The contents were mixed by hand to keep the cells suspended. 50 ml of starvation media was added as the media blank without cells. Five wells were the control cells without reagent. The next adjacent row of wells contained the lowest concentration of recombinant tissue protective cytokines. Each adjacent row of wells thereafter was filled with sequentially greater concentrations. The cell cultures that were incubated in media with 3% serum and without Epo were plated out at 200.000 cells/ml and 100 μl per well in 96-well plates. The contents were mixed briefly and carefully, using the orbital vibrating platform seated on top of the stir plate. The plate was incubated with different concentrations of Epo variants (from 0.2 pM to 20 nM) for 48 h in RPMI 1640 medium containing 3% serum in a humidified incubator with 5% CO₂ at 37° C. On day four of the assay, the 96-well plate was taken out from the Incubator and placed at room temperature in the laminar flow hood. lmmediately, the bioactivity is quantified (spectrophotometric absorption at 450 nm, subtracted from background absorption at 620 nm) by measuring the formazan product formed during cellular metabolism of the tetrazolium dye WST1, which correlates with cellular viability/number of cells.

Results

The UT7 cells showed stable and reliable growth in Epo containing media for 3 months.

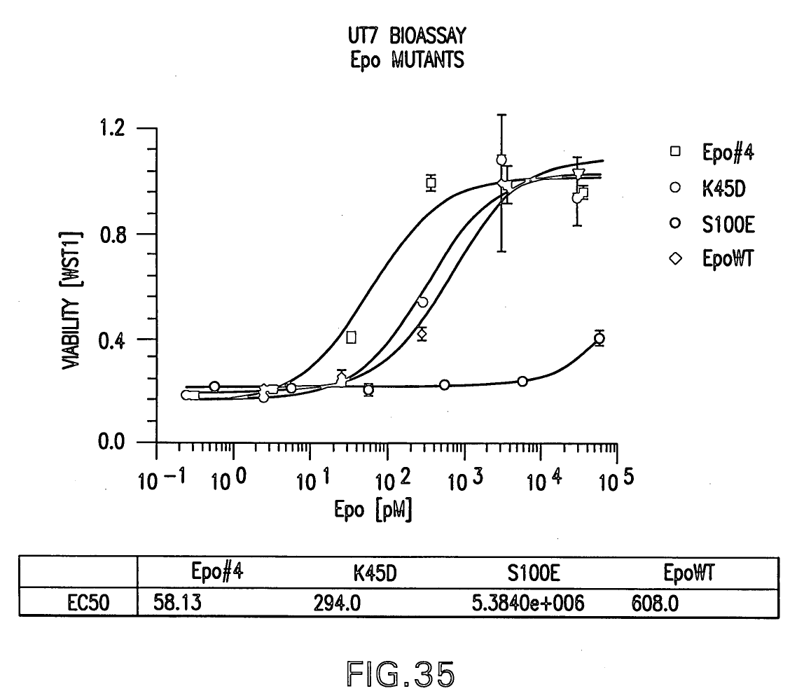

K45D induced a viability increase of the Epo-dependent UT-7 cells in a dose dependent way, with an EC₅₀ of 294.0. In comparison, the EC₅₀ was 58.13 for Epo (FIG. 35) and 608 for His-tagged Epo (EpoWT). S100E did not increase viability (more than 50%) of the Epo-dependent UT-7 cells at concentrations <50 nM (i.e. within the measurable range). Hence, K45D showed potency within the same order of magnitude as Epo, while S100E showed at least 1000-fold lower potency as compared to Epo.

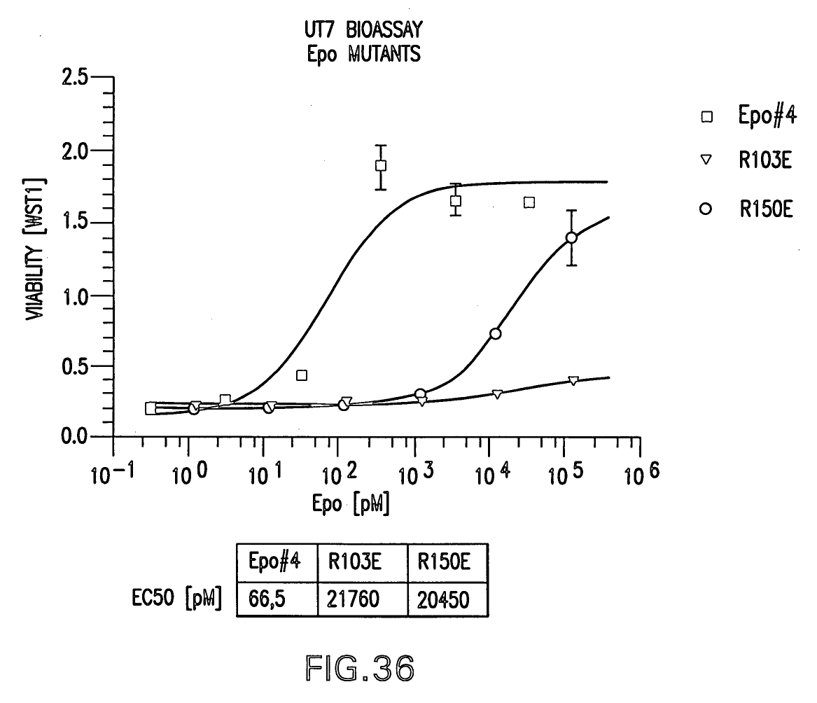

R103E did not increase survival of the Epo-dependent UT-7 cells at concentrations up to 20 nM, i.e. its potency compared to Epo was at least four orders of magnitude lower. R150E induced survival of the Epo-dependent UT-7 cells in a dose dependent way, with an EC₅₀ of 20 nM. In comparison, the EC₅₀ was 66.5 for Epo (Epo#4) (FIG. 36). Hence, R150E showed three orders of magnitude lower potency as compared to Epo.

FIG. 35 shows concentration-response curves of Epo, K45D and S100E in UT-7 cells. Different concentrations of Epo, EpoWT, K45D and S100E were added to UT-7 cells. Viability was measured 48 h later in the WST-1 assay. Data are mean ± SD of three different experiments each performed in duplicate. The curve is a non-linear regression curve fit.

FIG. 36 shows dose response curves of Epo, R103E and R150E in UT-7 cells. Different concentrations of Epo, EpoWT, R103E and R150E were added to UT-7 cells. Viability was measured 48 h later in the WST-1 assay. Data are mean ± SD of three different experiments each performed in duplicate. The curve is a non-linear regression curve fit.

### 6.18. Example 18

### Protection of Retinal Ischemia by Peripherally-Administered Recombinant Tissue Protective Cytokines

As described in Section 6.9, retinal cells are very sensitive to ischemia such that many will die after 30 minutes of ischemic stress. In this experiment, the rat reversible glaucoma model was again utilized as described by Rosenbaum et al. (1997; Vis. Res. 37:3443-51). The effects of recombinant tissue protective cytokines on ischemic stress were examined.

One eye in each of the rats was injured in accordance with the protocol outlined in the example presented in Section 6.9 for saline injection into the anterior chamber of the adult male rat eye. At the time of reperfusion, i.e. when the pressure in the anterior chamber of the eye is released, the rats were administered 10 μg/kg of EPO, one of four recombinant tissue protective cytokines: R103E, R150E, S100E, and S100e/K45D, or saline intravenously. On days 1, 3, 5 and 6 following the injury, electroretinograms were performed on both the injured and normal eye of each rat. The latency in the damaged eye of each rat was compared to the latency in the normal eye of the same rat. The data was recorded as a ratio of the latency of the injured eye over the latency the normal eye resulting in a ratio of one when the damaged eye has normal function. There are two components to the injury results: Amplitude (the difference from the peak to the trough as shown in FIG. 17, Panel A, indicated by ‘b’ and Latency, the time that it takes to achieve the peak in response to the stimulus.

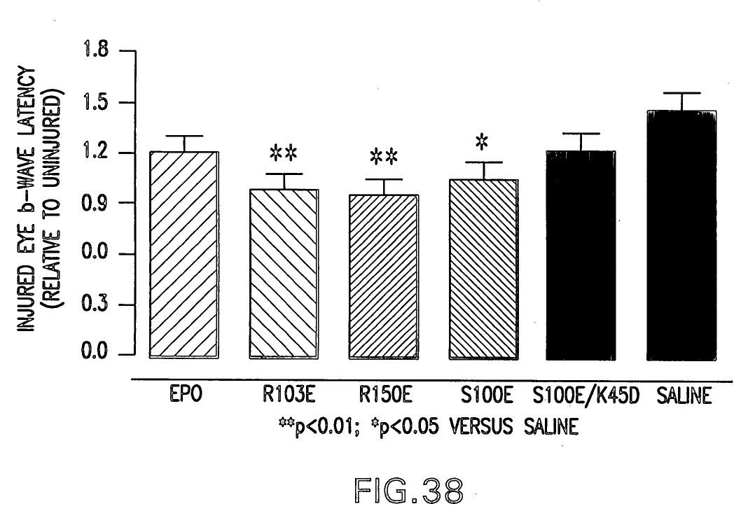

FIG. 38 shows the ratio of the latency of the injured eye over the latency the normal eye for the various treatment regimens. The rat treated with EPO exhibited a latency of 1.2, which is better than the rat treated with saline. Each of the four recombinant tissue protective cytokines resulted in latency results equal to or better than EPO with R103E, R150E, and S100E showing a statistical improvement over saline.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

What is claimed is:

1. A mutein recombinant tissue protective cytokine lacking at least one activity selected from the group consisting of increasing hematocrit, vasoactive action, hyperactivating platelets, pro-coagulant activities and increasing production of thrombocytes, the cytokine comprising at least one responsive cellular protective activity selected from the group consisting of protecting, maintaining, enhancing or restoring the function or viability of a responsive mammalian cell, tissue or organ.

2. The recombinant tissue protective cytokine of claim 1, comprising one or more altered amino acid residue between position 11 to 15 of SEQ ID NO:10 [SEQ ID NO:1], position 44 to 51 of SEQ ID NO 10 [SEQ ID NO:2], position 100-108 of SEQ ID NO [SEQ ID NO:3], or position 146-151 of SEQ ID NO 10 [SEQ ID NO:4].

3. The recombinant tissue protective cytokine of claim 1, comprising an altered amino acid residue at one or more of the following positions of SEQ ID NO:10: 7, 20, 21, 29, 33, 38, 42, 59, 63, 67, 70, 83, 96, 126, 142, 143, 152, 153, 155, 156, or 161.

4. The recombinant tissue protective cytokine of claim 1, comprising the amino acid sequence of SEQ ID NO:10 with one or more of the amino acid residue substitutions of SEQ ID NOs: 15-105 and 119.

5. The recombinant tissue protective cytokine of claim 1, comprising the amino acid sequence of SEQ ID NO:10 with a deletion of amino acid residues 44-49 of SEQ ID NO: 10.

6. The recombinant tissue protective cytokine of claim 1, comprising, the amino acid sequence of SEQ ID NO:10 with at least one of the following amino acid residue substitutions of SEQ ID NOs: 106-118.

7. The recombinant tissue protective cytokine of any one of claims 1, 2, 3, 4, 5, or 6, further comprising a chemical modification of one or more amino acids.

8. The recombinant tissue protective cytokine of claim 7, wherein the chemical modification comprises altering the charge of the recombinant tissue protective cytokine.

9. The recombinant tissue protective cytokine of claim 8, wherein a positive or negative charge is chemically added to an amino acid residue where a charged amino acid residue is modified to an uncharged residue.

10. The recombinant tissue protective cytokine of any one of claims 1, 2, 3, 4, 5, or 6, wherein said cytokine is a human erythropoietin mutein.

11. The recombinant tissue protective cytokine of any one of claims 1, 2, 3, 4, 5, or 6, wherein said cytokine is a human phenylglyoxal erythropoietin mutein.

12. The recombinant tissue protective cytokine of any one of claims 1, 2, 3, 4, 5, or 6, wherein the responsive mammalian cell comprises a neuronal, muscle, heart, lung, liver, kidney, small intestine, adrenal cortex, adrenal medulla, capillary, endothelial, testis, ovary, endometrial, or stem cell.

13. The recombinant tissue protective cytokine responsive mammalian cell of any one of claims 1, 2, 3, 4, 5, or 6, comprising a photoreceptor, ganglion, bipolar, horizontal, amacrine, Muieller, myocardium, pace maker, sinoatrial node, sinus node, atrioventricular node, bundle of His, hepatocyte, stellate, Kupffer, mesangial, goblet, intestinal gland, enteral endocrine, glomerulosa, fasciculate, reticularis, chromaffin, pericyte, Leydig, Sertoli, sperm, Graffian follicles, primordial follicles, endometrial stroma, and endometrial cell.

14. The recombinant tissue protective cytokine of any one of claims 1, 2, 3, 4, 5, or 6, wherein said cytokine is capable of traversing an endothelial cell barrier.

15. The recombinant tissue protective cytokine of claim 14, wherein the endothelial cell barrier comprises the blood-brain barrier, the blood-eye barrier, the blood testes barrier, the blood-ovary barrier, and the blood-uterus barrier.

16. The recombinant tissue protective cytokine of any one of claims 1, 2, 3, 4, 5, or 6, wherein said cytokine is selected from the group consisting of:
i. a cytokine having a reduced number or no sialic acid moieties;
ii. a cytokine having a reduced number or no N-linked or O-linked carbohydrates;
iii. a cytokine having at least a reduced carbohydrate content by virtue of treatment of native cytokine with at least one glycosidase;
iv. a cytokine having at least one or more oxidized carbohydrates;
v. a cytokine having at least one or more oxidized carbohydrates and is chemically reduced;
vi. a cytokine having at least one or more modified arginine residues;
vii. a cytokine having at least one or more modified lysine residues or a modification of the N-terminal amino group of a cytokine molecule;
viii. a cytokine having at least a modified tyrosine residue;
ix. a cytokine having at least a modified aspartic acid or glutamic acid residue;
x. a cytokine having at a modified tryptophan residue;
xi. a cytokine having at least one amino acid group removed;
xii. a cytokine having at least one opening of at least one of the cystine linkages in the cytokine molecule;
xiii. a truncated cytokine;
xiv. a cytokine having at least one polyethylene glycol molecule attached;
xv. a cytokine having at least one fatty acid attached;
xvi. a cytokine having a non-mammalian glycosylation pattern by virtue of the expression of a recombinant cytokine in non-mammalian cells; and
xvi. a cytokine having at least one histidine tagged amino acid to facilitate purification.

17. The recombinant tissue protective cytokine of claim 16 wherein said cytokine is an asialoerythropoietin.

18. The recombinant tissue protective cytokine of claim 17, wherein said asialoerythropoietin is human asialoerythropoietin.

19. The recombinant tissue protective cytokine of claim 16, wherein said cytokine is hyposialylated or hypersialylated.

20. The recombinant tissue protective cytokine of claim 16, wherein said cytokine comprises 1, 2, 3, 4, 5, 6, 7, 8, 9, 10, 11, 12, or 13 sialic acid moieties.

21. The recombinant tissue protective cytokine of claim 16, wherein said cytokine comprises more than the fourteen sialic acid moieties present in native erythropoietin.

22. The recombinant tissue protective cytokine of claim 16, wherein said cytokine is an erythropoietin with no N-linked carbohydrates.

23. The recombinant tissue protective cytokine of claim 22, wherein said cytokine is an erythropoietin with no O-linked carbohydrates.

24. The recombinant tissue protective cytokine of claim 16, wherein said cytokine is treated with at least one glycosidase.

25. The recombinant tissue protective cytokine of claim 16, wherein said cytokine is periodate-oxidized erythropoietin.

26. The recombinant tissue protective cytokine of claim 25, wherein said periodate-oxidized erythropoietin is chemically reduced with sodium cyanoborohydride.

27. The recombinant tissue protective cytokine of claim 16, wherein said cytokine comprises an R-glyoxal moiety on the one or more arginine residues, wherein R is aryl or alkyl moiety.

28. The recombinant tissue protective cytokine of claim 27, wherein said cytokine is phenylglyoxal-erythropoietin.

29. The recombinant tissue protective cytokine of claim 16, wherein said cytokine is an erythropoietin in which an arginine residue is modified by reaction with a vicinal diketone selected from the group consisting of 2,3-butanedione and cyclohexanedione.

30. The recombinant tissue protective cytokine of claim 16, wherein said cytokine is an erythropoietin in which an arginine residue is reacted with 3-deoxyglucosone.

31. The recombinant tissue protective cytokine of claim 16, wherein said cytokine is a molecule having at least one biotinylated lysine or N-terminal amino group.

32. The recombinant tissue protective cytokine of claim 16, wherein said cytokine is a glucitolyl lysine erythropoietin or fructosyl lysine erythropoietin.

33. The recombinant tissue protective cytokine of claim 16, wherein said cytokine comprises at least one carbamylated lysine residue.

34. The recombinant tissue protective cytokine of claim 33, wherein said carbamylated cytokine is comprised of alpha-N-carbamoylerythropoietin; N-epsilon-carbamoylerythropoietin; alpha-N-carbamoyl, N-epsilon-carbamoylerythropoietin; alpha-N-carbamoylasialoerythropoietin; N-epsilon-carbamoylasialoerythropoietin; alpha-N-carbamoyl, N-epsilon-carbamoylasialoerythropoietin; alpha-N-carbamoylhyposialoerythr-opoietin; N-epsilon-carbamoylhyposialoerythropoietin; and alpha-N-carbamoyl, N-epsilon-carbamoylhyposialoerythropoietin.

35. The recombinant tissue protective cytokine of claim 16, wherein said cytokine comprises at least one acylated lysine residue.

36. The recombinant tissue protective cytokine of claim 35, wherein said cytokine comprises at least one acylated lysine residue.

37. The recombinant tissue protective cytokine of claim 36, wherein said cytokine comprises at least one acylated lysine residue.

38. The recombinant tissue protective cytokine of claim 37, wherein a said acetylated cytokine is comprised of alpha-N-acetylerythropoietin; N-epsilon-acetylerythropoietin; alpha-N-acetyl, N-epsilon-acetylerythropoietin; alpha-N-acetylasialoerythropoietin; N-epsilon-acetylasialoerythropoietin; alpha-N-acetyl, N-epsilon-acetylasialoerythropoietin; alpha-N-acetylhyposialoerythropoietin; N-epsilon-acetylhyposialoerythropoietin; and alpha-N-acetyl, N-epsilon-acetylhyposialoerythropoietin.

39. The recombinant tissue protective cytokine of claim 35, wherein a lysine residue of said cytokine is succinylated.

40. The recombinant tissue protective cytokine of claim 39, wherein said succinylated cytokine is comprised of alpha-N-succinylerythropoietin; N-epsilon-succinylerythropoietin; alpha-N-succinyl, N-epsilon-succinylerythropoietin; alpha-N-succinylasialoerythropoietin; N-epsilon-succinylasialoerythropoietin; alpha-N-succinyl, N-epsilon-succinylasialoerythropoietin; alpha-N-succinylhyposialoerythropoietin; N-epsilon-succinylhyposialoerythropoietin; and alpha-N-succinyl, N-epsilon-succinylhyposialoerythropoietin.

41. The recombinant tissue protective cytokine of claim 16, wherein said cytokine comprises at least one lysine residue modified by 2, 4, 6 trintrobenzenesulfonate sodium or another salt thereof.

42. The recombinant tissue protective cytokine of claim 16, wherein said cytokine comprises at least one nitrated or iodinated tyrosine residue.

43. The recombinant tissue protective cytokine of claim 16, wherein said cytokine comprises an aspartic acid or glutamic acid residue that is reacted with a carbodiimide followed by reaction with an amine.

44. The recombinant tissue protective cytokine of claim 16, wherein a said amine is glycinamide.

45. An isolated nucleic acid molecule that comprises a nucleotide sequence which encodes a polypeptide comprising the recombinant tissue protective cytokine of any one of claims 1, 2, 3, 4, 5, or 6.

46. A vector comprising a nucleic acid molecule of claim 45.

47. An expression vector comprising a nucleic acid molecule of claim 45 and at least one regulatory region operably linked to the nucleic acid molecule.

48. The vector of claim 46 or 47 that is a pCiNeo vector.

49. A genetically-engineered cell which comprises a nucleic acid molecule of claim 45.

50. A cell comprising the expression vector of claim 45.

51. A pharmaceutical composition comprising a recombinant tissue protective cytokine of any one of claims 1, 2, 3, 4, 5, or 6, lacking at least one activity selected from the group consisting of increasing hematocrit, vasoactive action, hyperactivating platelets, pro-coagulant activities and increasing production of thrombocytes, the cytokine having at least one responsive cellular protective activity selected from the group consisting of protecting, maintaining, enhancing or restoring the function or viability of a responsive mammalian cell, tissue or organ.

52. The pharmaceutical composition of claim 51, formulated for oral, intranasal, or parenteral administration.

53. The pharmaceutical composition of claim 51, formulated as a perfusate solution.

54. A method for protecting, maintaining or enhancing the viability of a cell, tissue or organ isolated from a mammalian body comprising exposing said cell, tissue or organ to a pharmaceutical composition comprising a mutein recombinant tissue protective cytokine.

55. The method of claim 54, wherein the protection does not effect bone marrow.

56. A method for protecting, maintaining or enhancing the viability of a cell, tissue or organ isolated from a mammalian body comprising exposing said cell, tissue or organ to a pharmaceutical composition comprising a recombinant tissue protective cytokine of any one of claims 1, 2, 3, 4, 5, or 6, that lacks at least one activity selected from the group consisting of increasing hematocrit, vasoactive action, hyperactivating platelets, pro-coagulant activity and increasing production of thrombocytes.

57. Use of a recombinant tissue protective cytokine of any one of claims 1, 2, 3, 4, 5, or 6, that lacks at least one activity selected from the group consisting of increasing hematocrit, vasoactive action, hyperactivating platelets, pro-coagulant activity and increasing production of thrombocytes, for the preparation of a pharmaceutical composition for the protection against and prevention of a tissue injury as well as the restoration of and rejuvenation of tissue and tissue function in a mammal.

58. The use of claim 57, wherein the injury is caused by a seizure disorder, multiple sclerosis, stroke, hypotension, cardiac arrest, ischemia, myocardial infarction, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis, alcoholism, mood disorder, anxiety disorder, attention deficit disorder, autism, Creutzfeld-Jakob disease, brain or spinal cord trauma or ischemia, heart-lung bypass, chronic heart failure, macular degeneration, diabetic neuropathy, diabetic retinopathy, glaucoma, retinal ischemia, or retinal trauma.

59. A method for facilitating the transcytosis of a molecule across an endothelial cell barrier in a mammal comprising administration to said mammal a composition comprising said molecule in association with a recombinant tissue protective cytokine of any one of claims 1, 2, 3, 4, 5, or 6, lacking at least one activity selected from the group consisting of increasing hematocrit, increasing blood pressure, hyperactivating platelets, and increasing production of thrombocytes.

60. The method of claim 59, wherein said association is a labile covalent bond, a stable covalent bond, or a non-covalent association with a binding site for said molecule.

61. The method of claim 59, wherein said endothelial cell barrier is selected from the group consisting of the blood-brain barrier, the blood-eye barrier, the blood-testis barrier, the blood-ovary barrier, the blood-heart barrier, the blood-kidney barrier, and the blood-placenta barrier.

62. The method of claim 59, wherein said molecule is a receptor agonist or antagonist hormone, a neurotrophic factor, an antimicrobial agent, an antiviral agent, a radiopharmaceutical, an antisense oligonucleotide, an antibody, an immunosuppressant, a dye, a marker, or an anti-cancer drug.

63. A composition for transporting a molecule via transcytosis across an endothelial cell barrier comprising said molecule in association with a recombinant tissue protective cytokine, of any one of claims 1, 2, 3, 4, 5, or 6, lacking at least one activity selected from the group consisting of increasing hematocrit, vasoactive action, hyperactivating platelets, pro-coagulant activity and increasing production of thrombocytes.

64. The composition of claim 63, wherein said association is a labile covalent bond, a stable covalent bond, or a non-covalent association with a binding site for said molecule.

65. The composition of claim 63, wherein said molecule is a receptor agonist or antagonist hormone, a neurotrophic factor, an antimicrobial agent, a radiopharmaceutical, an antisense oligonucleotide, an antibody, an immunosuppressant, a dye, a marker, or an anti-cancer drug.

66. Use of an recombinant tissue protective cytokine of any one of claims 1, 2, 3, 4, 5, or 6, lacking at least one activity selected from the group consisting of increasing hematocrit, vasoactive action, hyperactivating platelets, pro-coagulant activities and increasing production of thrombocytes.

67. The use of claim 66, wherein said association is a labile covalent bond, a stable covalent bond, or a non-covalent association with a binding site for said molecule.

68. The use of claim 66, wherein said molecule is a receptor agonist or antagonist hormone, a neurotrophic factor, an antimicrobial agent, a radiopharmaceutical, an antisense oligonucleotide, an antibody, an immunosuppressant, a dye, or a marker, or an anti-cancer drug.
